(19) United States
(12) Patent Application Publication
(10) Pub. No.: US 2026/0199525 A1
(43) Pub. Date: Jul. 16, 2026

(54) ENGINEERED CHIMERIC FUSION PROTEIN COMPOSITIONS AND METHODS OF USE THEREOF

(71) Applicant: CREATE Medicines, Inc., Cambridge, MA (US)

(72) Inventors: Michele Luise GERBER, Ann Arbor, MI (US); Matthew Anthony MAURER, Princeton, NJ (US); Daniel GETTS, Medfield, MA (US); Yuxiao WANG, Belmont, MA (US)

(21) Appl. No.: 19/425,038

(22) Filed: Dec. 18, 2025

Related U.S. Application Data
(63) Continuation of application No. PCT/US2024/036254, filed on Jun. 28, 2024.
(60) Provisional application No. 63/511,274, filed on Jun. 30, 2023.
(60) Provisional application No. 63/579,422, filed on Aug. 29, 2023.

(51) Int. Cl. A61K 48/00; A61K 38/17; A61K 39/395; A61K 45/06; A61P 35/00

(52) U.S. Cl. CPC A61K 48/005; A61K 38/1774; A61K 39/3955; A61K 45/06; A61K 48/0033; A61P 35/00

(57) ABSTRACT

Compositions and methods for making and using engineered cells, such as, engineered myeloid cells that express a chimeric fusion protein that has a binding domain capable to binding surface molecules on target cells such as diseased cells.

### CROSS REFERENCE

This application is a continuation application of International Application No. PCT/US2024/036254 filed Jun. 28, 2024, which claims the benefit of U.S. Provisional Application No. 63/511,274, filed on Jun. 30, 2023; and U.S. Provisional Application No. 63/579,422, filed on Aug. 29, 2023; each of which is incorporated herein by reference in its entirety.

### SEQUENCE LISTING

The instant application contains a Sequence Listing which has been submitted electronically in XML format and is hereby incorporated by reference in its entirety. Said XML copy, created on Aug. 28, 2024, is named 56371-754_601_SL.xml and is 60,426 bytes in size.

### BACKGROUND

Cellular immunotherapy is a promising new technology for fighting difficult to treat diseases, such as cancer, and persistent infections and also certain diseases that are refractory to other forms of treatment. A major breakthrough has come across with the discovery of CAR-T cell and their potential use in immunotherapy. CAR-T cells are T lymphocytes expressing a chimeric antigen receptor which helps target the T cell to specific diseased cells such as cancer cells, and can induce cytotoxic responses intended to kill the target cancer cell or immunosuppression and/or tolerance depending on the intracellular domain employed and co-expressed immunosuppressive cytokines. Although CAR-T cells continue to remain prospective tools for cancer therapy, several limitations along the way has slowed the progress on CAR-T cells and dampened its promise in clinical trials.

Understanding the limitations of CAR-T cells is the key to leveraging the technology and continue innovations towards better immunotherapy models. Specifically, in T cell malignancies, CAR-T cells appear to have faced a major problem. CAR-T cells and malignant T cells share surface antigen in most T cell lymphomas (TCL), therefore, CAR-T cells are subject to cytotoxicity in the same way as cancer cells. In some instances, the CAR-T products may be contaminated by malignant T cells. Additionally, T cell aplasia is a potential problem due to prolonged persistence of the CAR-T cells. Other limitations include the poor ability for CAR-T cells to penetrate into solid tumors and the potent tumor microenvironment which acts to downregulate their anti-tumor potential. CAR-T cell function is also negatively influenced by the immunosuppressive tumor microenvironment (TME) that leads to endogenous T cell inactivation and exhaustion.

Myeloid cells, including macrophages, are cells derived from the myeloid lineage and belong to the innate immune system. They are derived from bone marrow stem cells which egress into the blood and can migrate into tissues. Some of their main functions include phagocytosis, the activation of T cell responses, and clearance of cellular debris and extracellular matrices. They also play an important role in maintaining homeostasis, and initiating and resolving inflammation. Moreover, myeloid cells can differentiate into numerous downstream cells, including macrophages, which can display different responses ranging from pro-inflammatory to anti-inflammatory depending on the type of stimuli they receive from the surrounding microenvironment. Furthermore, tissue macrophages have been shown to play a broad regulatory and activating role on other immune cell types including CD8+ and CD4+ T effector cells, NK cells and T regulatory cells. Macrophages have been shown to be a main immune infiltrate in malignant tumors and have been shown to have a broad immunosuppressive influence on effector immune infiltration and function.

### SUMMARY

The diverse functionality of myeloid cells makes them an ideal cell therapy candidate that can be engineered to have numerous therapeutic effects. The present disclosure is related to immunotherapy using myeloid cells (e.g., CD14+ cells) of the immune system, particularly phagocytic cells. A number of therapeutic indications could be contemplated using myeloid cells. For example, myeloid cell immunotherapy could be exceedingly important in treating cancer, autoimmunity, fibrotic diseases and infections. The present disclosure is related to immunotherapy using myeloid cells, including phagocytic cells of the immune system, particularly monocytes. It is an object of the invention disclosed herein to harness one or more of these functions of myeloid cells for therapeutic uses. For example, it is an object of the invention disclosed herein to harness the phagocytic activity of myeloid cells, including engineered myeloid cells, for therapeutic uses. For example, it is an object of the invention disclosed herein to harness the ability of myeloid cells, including engineered myeloid cells, to promote T cell activation. For example, it is an object of the invention disclosed herein to harness the ability of myeloid cells, including engineered myeloid cells, to promote secretion of tumoricidal molecules. For example, it is an object of the invention disclosed herein to harness the ability of myeloid cells, including engineered myeloid cells, to promote recruitment and trafficking of immune cells and molecules. In one aspect the disclosure provides new and useful chimeric constructs that, when expressed in a myeloid cell, the myeloid cell can drive targeted attack and phagocytosis of the molecule, molecular assembly, object or a cell that comprises the target, e.g., a target antigen on its surface. One of the many facets of the present disclosure is to (i) enhance the phagocytic ability of the myeloid cells (e.g., the engineered myeloid cells expressing the new and improved chimeric constructs); help initiate a coordinated and sustained immune response against the target (e.g., target antigen). The present disclosure provides innovative methods and compositions that can successfully transfect or transduce a myeloid cell, or otherwise induce a genetic modification in a myeloid cell, with the purpose of augmenting a functional aspect of a myeloid cell, additionally, without compromising the cell's differentiation capability, maturation potential, and/or its plasticity. The resultant cells may be termed therapeutically effective engineered myeloid cells, or effector myeloid cells. One strategy for improvement described herein is to induce an inflammatory phenotype of the myeloid cells to develop effector myeloid cells. One strategy is to generate effector myeloid cells capable of mounting an inflammatory phenotype upon engagement with the target.

Provided herein is a composition comprising a recombinant polynucleotide, wherein the recombinant polynucleotide comprises a sequence encoding a chimeric fusion protein (CFP), the CFP comprising: (i) an extracellular domain comprising a TROP2 antigen binding domain, (ii) a transmembrane domain operatively linked to the extracellular domain; and (iii) optionally, an intracellular domain; wherein the transmembrane domain binds to a transmembrane domain of an endogenous FcR receptor when the mRNA is expressed in a cell; wherein upon binding of the transmembrane domain encoded by the mRNA to the endogenous FcR receptor the CFP encoded by the mRNA is expressed on the surface of the cell.

In some embodiments, the antigen binding domain has an HCDR1, HCDR2, HCDR3, LCDR1, LCDR2 and LCDR3 of an antigen binding domain of Table 1A.

In some embodiments, the antigen binding domain has a VH with at least 80% sequence identity to a VH of an antigen binding domain of Table 1A, and a VL with at least 80% sequence to a VL of an antigen binding domain of Table 1A.

In some embodiments, the CFP further comprises a signal peptide.

In some embodiments, the signal peptide has a sequence according to a signal peptide of Table 1B.

In some embodiments, the extracellular domain comprises a sequence with at least 80% sequence identity to a sequence of Table 1C.

In some embodiments, the transmembrane domain comprises a sequence with at least 80% sequence identity to a sequence of Table 1D.

In some embodiments, the each of the intracellular signaling domains of the at least two intracellular signaling domains has a sequence with at least 80% sequence identity to a sequence of Table 2.

In some embodiments, the intracellular domain has a sequence with at least 80% sequence identity to a sequence of Table 3.

In some embodiments, the CFP has a sequence with at least 80% sequence identity to a sequence of Table 4.

Also provided herein is a composition comprising a recombinant polynucleotide, wherein the recombinant polynucleotide comprises a sequence encoding a chimeric fusion protein (CFP), wherein the CFP has a sequence with at least 95% sequence identity to a sequence of Table 4.

In some embodiments, the CFP has a sequence with at least 98%, 99% or 100% sequence identity to a sequence of Table 4.

In some embodiments, the intracellular domain comprises at least one additional intracellular signaling domain.

In some embodiments, the antigen binding domain comprises an antibody or a fragment thereof.

In some embodiments, the antigen binding domain comprises an scFv.

In some embodiments, the antigen binding domain comprises the extracellular domain comprises a hinge domain connecting the antigen binding domain and the transmembrane domain.

In some embodiments, the recombinant polynucleotide is an mRNA.

In some embodiments, the recombinant polynucleotide is associated with one or more lipids.

In some embodiments, the recombinant polynucleotide is encapsulated in a liposome.

In some embodiments, the liposome is a lipid nanoparticle.

Provided herein is a pharmaceutical product comprising a recombinant mRNA encoding a chimeric antigen receptor comprising: an anti-TROP2 binding scFv extracellular domain; and a CD89 transmembrane domain; wherein the pharmaceutical product is formulated in an aqueous formulation for delivery systemically. In some embodiments, the pharmaceutical composition comprises an scFv comprising a heavy chain and a light chain, the heavy chain comprising a CDR3 having a sequence GGFGSSYWYFDV, and the light chain comprising a CDR3 having a sequence QQHYITPLT. In some embodiments, the heavy chain further comprises a CDR1 sequence of NYGMN, and a CDR2 sequence of WINTYTGEPTYTDDFKG; and the light chain further comprises a CDR1 sequence of KASQDVSIAVA, and a CDR2 sequence of SASYRYT. The pharmaceutical composition described above comprises a CD89 transmembrane domain having a sequence LIRMAVAGLVLVALLAILV.

In some embodiments, the recombinant polynucleotide comprises a vector.

Also provided herein is a composition comprising a nanoparticle delivery vehicle and a recombinant polynucleotide described herein, wherein the recombinant polynucleotide is associated with or within the nanoparticle delivery vehicle.

In some embodiments, the transmembrane domain is a transmembrane domain from a protein that dimerizes with endogenous FcR-gamma receptors in myeloid cells.

In some embodiments, the transmembrane domain comprises a transmembrane domain from CD16a, CD64, CD68 or CD89.

In some embodiments, the recombinant polynucleotide is associated with or within a nanoparticle delivery vehicle, wherein the nanoparticle delivery vehicle comprises a lipid nanoparticle or a polymeric nanoparticle.

In some embodiments, the recombinant polynucleotide is an mRNA and the nanoparticle encapsulates the mRNA.

Also provided herein is a composition comprising a cell comprising a recombinant polynucleotide described herein.

In some embodiments, the cell is an immune cell.

In some embodiments, the cell is a myeloid cell, a lymphoid cell, a precursor cell, a stem cell or an induced pluripotent cell.

In some embodiments, the cell is CD14+CD16− cell.

Also provided herein is a pharmaceutical composition comprising a composition described herein; and a pharmaceutically acceptable excipient.

Also provided herein is a method of treating a cancer in a subject comprising: administering to the subject a pharmaceutical composition described herein.

In one aspect, provided herein is a pharmaceutical composition comprising of a polynucleotide encoding a chimeric antigen receptor comprising: an anti-TROP2 binding scFv extracellular domain; and a CD89 transmembrane domain formulated in an aqueous formulation for delivery systemically, at a therapeutically effective amount and time interval suitable for treating a cancer in a subject. In some embodiments, the subject is a human subject. In some embodiments, the polynucleotide is an engineered RNA. In some embodiments, the polynucleotide is an engineered mRNA. In some embodiments, the anti-TROP2 binding scFv comprises a heavy chain, and a light chain, wherein the heavy chain comprises a CDR3 having a sequence GGFGSSYWYFDV, and the light chain comprises a CDR3 having a sequence QQHYITPLT. In some embodiments, the heavy chain further comprises a CDR1 sequence of NYGMN, and a CDR2 sequence of WINTYTGEPTYTDDFKG; and the light chain further comprises a CDR1 sequence of KASQDVSIAVA, and a CDR2 sequence of SASYRYT.

In some embodiments, the CD89 transmembrane domain comprises a sequence LIRMAVAGLVLVALLAILV.

In some embodiments, the pharmaceutical composition further comprises a lipid nanoparticle delivery vehicle. In some embodiments, the lipid nanoparticle delivery vehicle comprises a cationic lipid, a non-cationic lipid, a neutral lipid, a PEGylated lipid, or a combination thereof. In some embodiments, the subject is a human subject. In some embodiments, the cancer is an epithelial cancer. In some embodiments, the subject has an epithelial cancer. In some embodiments, the cancer is selected from Urothelial, Cervical, Ovarian epithelial, Triple-negative breast, HR+/HER2− breast, Pancreatic ductal adenocarcinoma, Gastric adenocarcinoma, Esophageal carcinoma, Non-small cell lung and Colorectal cancer.

In some embodiments, the cancer is metastatic.

In some embodiments, the pharmaceutical composition is formulated for systemic delivery.

In some embodiments, the pharmaceutical composition is formulated for intravenous delivery.

In some embodiments, the pharmaceutical composition is formulated for an intravenous injection or an infusion.

In some embodiments, the therapeutically effective amount of a dose comprises 1-5000 microgram/microliter of the engineered RNA.

In some embodiments, the pharmaceutical composition comprises an amount of about 0.0005 to about 0.001, about 0.001 to about 0.005, about 0.005 to about 0.01, about 0.01 to about 0.05, about 0.05 to about 0.1, or about 0.1 to about 0.5 mg/kg of the engineered RNA per dose of the pharmaceutical composition.

In some embodiments, the pharmaceutical composition comprises an amount of about 0.001 to about 0.0015, about 0.0015 to about 0.002, about 0.002 to about 0.0025, about 0.0025 to about 0.003, about 0.003 to about 0.0035, about 0.0035 to about 0.004, about 0.004 to about 0.0045, about 0.0045 to about 0.005, about 0.005 to about 0.0055, about 0.0055 to about 0.006, about 0.006 to about 0.0065, about 0.0065 to about 0.007, about 0.007 to about 0.0075, about 0.0075 to about 0.008, about 0.008 to about 0.0085, about 0.0085 to about 0.009, about 0.009 to about 0.0095, or about 0.0095 to about 0.01 mg of the engineered RNA per kg of the subject's body weight (mg/kg) per dose of the pharmaceutical composition.

In some embodiments, the pharmaceutical composition comprises an amount of about 0.01 to about 0.015, about 0.015 to about 0.02, about 0.02 to about 0.025, about 0.025 to about 0.03, about 0.03 to about 0.035, about 0.035 to about 0.04, about 0.04 to about 0.045, about 0.045 to about 0.05, about 0.05 to about 0.055, about 0.055 to about 0.06, about 0.06 to about 0.065, about 0.065 to about 0.07, about 0.07 to about 0.075, about 0.075 to about 0.08, about 0.08 to about 0.085, about 0.085 to about 0.09, about 0.09 to about 0.095, or about 0.095 to about 0.1 mg/kg of the engineered RNA per dose of the pharmaceutical composition.

In some embodiments, the pharmaceutical composition comprises an amount of about 0.001, about 0.0015, about 0.002, about 0.0025, about 0.003, about 0.0035, about 0.004, about 0.0045, about 0.005, about 0.0055, about 0.006, about 0.0065, about 0.007, about 0.0075, about 0.008, about 0.0085, about 0.009, about 0.0095, or about 0.01 mg/kg of the engineered RNA per dose of the pharmaceutical composition.

In some embodiments, the pharmaceutical composition comprises a dose of about 0.01, about 0.015, about 0.02, about 0.025, about 0.03, about 0.035, about 0.04, about 0.045, about 0.05, about 0.055, about 0.06, about 0.065, about 0.07, about 0.075, about 0.08, about 0.085, about 0.09, about 0.095, or about 0.1 mg/kg of the engineered RNA per dose of the pharmaceutical composition.

In some embodiments, the pharmaceutical composition comprises an amount of at least about 0.001, at least about 0.0015, at least about 0.002, at least about 0.0025, at least about 0.003, at least about 0.0035, at least about 0.004, at least about 0.0045, at least about 0.005, at least about 0.0055, at least about 0.006, at least about 0.0065, at least about 0.007, at least about 0.0075, at least about 0.008, at least about 0.0085, at least about 0.009, at least about 0.0095, or at least about 0.01 mg/kg of the engineered RNA per dose of the pharmaceutical composition.

In some embodiments, the pharmaceutical composition comprises an amount of at least about 0.01, at least about 0.015, at least about 0.02, at least about 0.025, at least about 0.03, at least about 0.035, at least about 0.04, at least about 0.045, at least about 0.05, at least about 0.055, at least about 0.06, at least about 0.065, at least about 0.07, at least about 0.075, at least about 0.08, at least about 0.085, at least about 0.09, at least about 0.095, or at least about 0.1 mg/kg, or at least about 0.2 mg/kg, or at least about 0.3 mg/kg, or at least about 0.4 mg/kg, or at least about 0.5 mg/kg, or at least about 0.6 mg/kg, or at least about 0.7 mg/kg, or at least about 0.8 mg/kg, or at least about 0.9 mg/kg, or at least about 1 mg/kg of the engineered RNA per dose of the pharmaceutical composition.

In some embodiments, the pharmaceutical composition comprises a concentration of the engineered RNA is between about 0.5 to about 1.5 mg/mL or between about 0.7 to about 1.3 mg/mL when stored in a container.

In some embodiments, the container is a single-use or multi-use vial.

In some embodiments, a total volume of the aqueous formulation is from about 1.0 to about 3.0 mL, from about 1.5 to about 2.5 mL, or about 2.0 mL.

In some embodiments, the dosing interval is 14 days for 3 doses, followed an interval of 28 days for three doses.

In one aspect, provided herein is a method of treating a cancer in a subject comprising: administering to the subject the pharmaceutical composition of any one of embodiments described above.

In some embodiments, the subject is a subject who is over 18 years of age.

In some embodiments, the subject exhibited progressive disease at baseline, or a refractory disease or a relapse in response to standard of care.

In some embodiments, the subject is not pregnant or donating sperm.

In some embodiments, the subject does not have CNS metastasis or carcinomatous meningitis.

In some embodiments, the pharmaceutical composition or the method of treating described herein comprises, reducing a tumor by at least 1%, 2%, 3%, 4%, 5%, 6%, 7%, 8%, 9% or at least 10% after the treatment.

In some embodiments, the pharmaceutical composition or the method of treating described herein comprises alleviating at least one of the symptoms associated with the cancer.

In some embodiments, the pharmaceutical composition comprises, an engineered RNA encapsulated in a lipid nanoparticle, wherein the engineered RNA comprises a sequence encoding a chimeric antigenic receptor (CAR) having an extracellular antigen binding domain comprising an scFv that binds to TROP2, having a heavy chain that comprises a CDR1 sequence of NYGMN, a CDR2 sequence of WINTYTGEPTYTDDFKG and a CDR3 sequence of GGFGSSYWYFDV; and the light chain further comprises a CDR1 sequence of KASQDVSIAVA, a CDR2 sequence of SASYRYT, and a CDR3 sequence of QQHYITPLT, a transmembrane domain and an intracellular domain from CD89, having a sequence DSIHQDYTTQNLIRMAVAGLVLVALLAILVENWHSHTALNKEASADVAEPSWSQQMCQPGLTFARTPSVCK, wherein the pharmaceutical composition has no dose limiting toxicities (DLT) at a dose of at least up to 0.03 mg/kg in a human subject in need thereof, when administered at a dosing regimen that is at least twice weekly for about 18 weeks.

In some embodiments, the extracellular antigen binding domain comprises a sequence that is at least 90%, identical to the sequence

(SEQ ID NO: 3)
QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYFDVWGQGSLVTVSSGGGGSGGGGSGGGGSDIQLTQSPSSL
SASVGDRVSITCKASQDVSIAVAWYQQKPGKAPKLLIYSASYRYTGVPD
RFSGSGSGTDFTLTISSLOPEDFAVYYCQQHYITPLTFGAGTKVEIKR.

In some embodiments, the engineered CAR comprises a sequence that is at least 95% identical to the sequence in SEQ ID NO: 3.

In some embodiments, the engineered CAR comprises a sequence that is at least 95% identical to the sequence in SEQ ID NO: 3.

In some embodiments, the engineered CAR comprises a sequence that is at least 99% identical to the sequence in SEQ ID NO: 3.

In some embodiments, the engineered CAR comprises a sequence that is at least 90% identical to any one of the sequences of SEQ ID NO: 26 and SEQ ID NO: 41.

In some embodiments, the engineered CAR comprises a sequence that is at least 90% identical to the sequences of SEQ ID NO: 27 and SEQ ID NO: 34.

In some embodiments, the engineered CAR comprises a sequence that is at least 90% identical to the sequences of SEQ ID NO: 28 and SEQ ID NO: 35.

In some embodiments, the engineered CAR comprises a sequence that is at least 90% identical to the sequences of SEQ ID NO: 29 and SEQ ID NO: 36.

In some embodiments, the engineered CAR comprises a sequence that is at least 90% identical to the sequences of SEQ ID NO: 30 and SEQ ID NO: 37.

In some embodiments, the engineered CAR comprises a sequence that is at least 90% identical to the sequences of SEQ ID NO: 31 and SEQ ID NO: 38.

In some embodiments, the engineered CAR comprises a sequence that is at least 90% identical to the sequences of SEQ ID NO: 32 and SEQ ID NO: 39.

In some embodiments, the engineered CAR comprises a sequence that is at least 90% identical to the sequences of SEQ ID NO: 33 and SEQ ID NO: 40.

In some embodiments, the engineered CAR comprises a sequence that is at least 95% identical to any one of the sequences of SEQ ID NOs: 26-41.

In some embodiments, the method of treating further comprises administering to the subject one or more therapeutic agents in combination with or in addition to administering the pharmaceutical composition.

In some embodiments, the administering of the one or more therapeutic agents comprises administering a therapeutic agent prior to administering the pharmaceutical composition.

In some embodiments, the administering of the one or more therapeutic agents comprises administering a therapeutic agent concomitantly with administering the pharmaceutical composition.

In some embodiments, the administering of the one or more therapeutic agents comprises administering a therapeutic agent following administering the pharmaceutical composition.

In some embodiments, the method comprises administering the pharmaceutical composition to the subject for at least one cycle. In some embodiments, the method comprises administering the pharmaceutical composition to the subject for at least 2, 3, or 4 cycles. In some embodiments, the pharmaceutical composition is administered to the subject about once a week, about once every 2 weeks, about once every 3 weeks, about once every 4 weeks, about once every 5 weeks, about once every 6 weeks, about once every 7 weeks, about once every 8 weeks, or about once every 9 weeks during a first cycle. In some embodiments, the method comprises administering the pharmaceutical composition to the subject once a week or once every 2 weeks during the first cycle.

In some embodiments, the method comprises administering the pharmaceutical composition to the subject at least 1, at least 2, at least 3, at least 4, at least 5, at least 6, at least 7, at least 8, at least 9, at least 10, at least 11, or at least 12 times for each cycle.

In some embodiments, the method comprises administering the pharmaceutical composition to the subject from about 1 to about 3, about 3 to about 6, about 6 to about 9, or about 9 to about 12 times for each cycle.

In some embodiments, the method comprises administering the pharmaceutical composition to the subject at most 1, at most 2, at most 3, at most 4, at most 5, at most 6, at most 7, at most 8, at most 9, at most 10, at most 11, or at most 12 times for each cycle.

In some embodiments, the method comprises administering the pharmaceutical composition to the subject between 1 to 12 times for each cycle.

In some embodiments, the method comprises administering the pharmaceutical composition to the subject between about 1 to about 3, about 3 to about 6, about 6 to about 9, or about 9 to about 12 times for each cycle. In some embodiments, the method comprises administering the pharmaceutical composition to the subject 3 times in a first cycle. In some embodiments, a second cycle follows a first cycle.

In some embodiments, the method further comprises administering the pharmaceutical composition to the subject during the second cycle. In some embodiments, the pharmaceutical composition is administered to the subject about once a week, about once every 2 weeks, about once every 3 weeks, about once every 4 weeks, about once every 5 weeks, about once every 6 weeks, about once every 7 weeks, about once every 8 weeks, or about once every 9 weeks during the second cycle. In some embodiments, the method comprises administering the pharmaceutical composition to the subject once every 4 weeks during the second cycle. In some embodiments, the method comprises administering the pharmaceutical composition to the subject at least 1, at least 2, at least 3, at least 4, at least 5, at least 6, at least 7, at least 8, at least 9, at least 10, at least 11, or at least 12 times in the second cycle. In some embodiments, the method comprises administering the pharmaceutical composition to the subject about 1 to about 3, about 3 to about 6, about 6 to about 9, or about 9 to about 12 times in the second cycle. In some embodiments, the method comprises administering the pharmaceutical composition to the subject at most 1, at most 2, at most 3, at most 4, at most 5, at most 6, at most 7, at most 8, at most 9, at most 10, at most 11, or at most 12 times in the second cycle. In some embodiments, the method comprises administering the pharmaceutical composition to the subject between 1 to 12 times in the second cycle. In some embodiments, the method comprises administering the pharmaceutical composition to the subject between about 1 to about 3, about 3 to about 6, about 6 to about 9, or about 9 to about 12 times in the second cycle. In some embodiments, the method comprises administering the pharmaceutical composition to the subject 3 times in the second cycle. In some embodiments, the method comprises administering the pharmaceutical composition wherein the therapeutically effective dose comprises an amount of 0.01 mg/kg to 1 mg/kg per dose in an interval of once every two weeks to once every week for about 18 weeks to about 50 weeks.

In one aspect, provided herein is a pharmaceutical composition, comprising an engineered mRNA comprising a sequence encoding the sequence of SEQ ID NO: 41, and wherein the pharmaceutical composition comprises a dose of the engineered mRNA from about 0.005 to about 0.15 mg/kg, wherein the dose is measured based on the weight of a subject. In some embodiments, the pharmaceutical composition comprises a dose of the engineered mRNA about 0.005, 0.015, 0.03, 0.06, 0.10, or 0.15 mg/kg. In some embodiments, the engineered mRNA is encapsulated in a lipid nanoparticle.

In one aspect, provided herein is a method for treating a cancer in a subject in need thereof, comprising administering a pharmaceutical composition comprising an engineered mRNA comprising a sequence encoding the sequence of SEQ ID NO: 41, at a dose of the engineered mRNA from about 0.005 to about 0.15 mg/kg, wherein the dose is measured based on the weight of the subject.

In one embodiment, provided herein is a method for treating a cancer in a subject in need thereof, comprising administering a pharmaceutical composition comprising an engineered mRNA comprising a sequence encoding the sequence that has at least 95% identity to the sequence of SEQ ID NO: 41, at a dose of the engineered mRNA from about 0.005 to about 0.15 mg/kg, wherein the dose is measured based on the weight of the subject. In some embodiments, the engineered mRNA comprises a sequence encoding the sequence that has at least 96%, 97%, 98% or 99% identity to the sequence of SEQ ID NO: 41, or a sequence that does not have a signal peptide sequence. In some embodiments, the ScFv VH and VL are from a humanized murine antibody. In some embodiments, the scFv is from a fully human antibody.

In one aspect, provided herein is a method for treating a cancer in a subject in need thereof, comprising administering a pharmaceutical composition comprising an engineered mRNA comprising a sequence encoding the sequence of SEQ ID NO: 26, at a dose of the engineered mRNA from about 0.005 to about 0.15 mg/kg, wherein the dose is measured based on the weight of the subject; or a sequence has at least 95%, 96%, 97%, 98% or 99% identity to the sequence of SEQ ID NO: 26.

In some embodiments, the engineered mRNA is encapsulated in a lipid nanoparticle. The lipid nanoparticle may comprise a plurality of lipids, that may usually be denoted herein as lipid 1 or lipid 2 and so forth, wherein at least one lipid is a polar lipid, and the other is at least a non-polar lipid.

In some embodiments, the dose of the engineered mRNA is about 0.005, 0.015, 0.03, 0.06, 0.10, or 0.15 mg/kg.

In some embodiments, the pharmaceutical composition is administered to the subject once every 7 days, once every 14 days, or once every 28 days.

In some embodiments, the pharmaceutical composition is administered to the subject for at least 4 cycles. In some embodiments, the pharmaceutical composition is administered at a dose of about 0.05, 0.15, or 0.03 mg/kg, and wherein the pharmaceutical composition is administered every 14 days at Cycle 1 and every 28 days at Cycles 2-4. In some embodiments, the pharmaceutical composition is administered at a dose of about 0.03 mg/kg, and wherein the pharmaceutical composition is administered every 7 days at Cycle 1 and every 28 days at Cycles 2-4.

In some embodiments, the pharmaceutical composition is administered at a dose of about 0.06, 0.10, or 0.15 mg/kg, and wherein the pharmaceutical composition is administered every 14 days. In some embodiments, a cycle comprises at least 28 days, 42 days, or 56 days.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 depicts an exemplary diagrammatic view of a chimeric antigen receptor (CAR) polypeptide construct for in vivo delivery, the CAR comprising a cancer cell-specific extracellular antigen binding domain comprising an scFv that has antigen binding specificity to a target cancer antigen, e.g. TROP2 antigen. The extracellular antigen binding domain (e.g., comprising scFv) is operatively linked to a CD89 transmembrane domain (TM) and CD89 cytoplasmic (Cyto) domain. A chimeric protein such as exemplified in the figure is referred as anti-TROP2-CD89 CAR, (interchangeably indicated as anti-TROP2 chimeric fusion protein (CFP)) when the antigen binding domain is a TROP2 binding domain. The figure demonstrates a cross section of a cell membrane expressing the CAR polypeptide, where the CAR transmembrane domain associates with an endogenous FcR gamma chain that stabilizes its expression in the cell.

FIG. 2 depicts inhibition of tumor growth in mice with gp75+ tumor, following intravenous administration of an mRNA in a lipid nanoparticle (LNP) (mRNA-LNP) composition wherein the mRNA encodes an anti-GP75-CD89 CAR. The anti-GP75-CD89 CAR is similar to the anti-TROP2-CD89 CAR represented graphically in FIG. 1, expect for the extracellular anti-TROP2 domain, which is swapped for an anti-GP75 domain. Both 0.5 mg/kg and 2 mg/kg showed tumor regression. The administration schedule of the anti-GP75-CD89 CAR expressing mRNA in the LNP formulation (anti-GP75-CD89 CAR-LNP) is indicated below the X axis with arrows.

FIG. 3 depicts expression of the anti-GP75 CFP in tumors following the intravenous administration of the mRNA-LNP composition of the construct depicted in FIG. 1.

FIGS. 4A-4C depict T cell activation in mouse GP75 tumor model. These mice develop GP75+ tumor. The mice were administered mRNA-LNP formulation, wherein the mRNA encodes the anti-GP75 CAR, having an extracellular anti-GP75 domain operatively linked to a CD89 transmembrane domain (TM) and CD89 cytoplasmic (Cyto) domain. FIG. 4A (left graph) shows dot plot depicting activation and exhaustion (TIM3/PD-1) of T cells isolated from mice post-treatment, showing increased cells triple positive for markers CD8+ TIM3+ and PD1+ in the CAR expressing cells from mice treated with the CAR construct compared to empty LNP by flow cytometry. FIG. 4A (right) shows a bar graph of the data shown in dot plot on the left figure. FIG. 4B shows dot plot (left) and bar graph (right) depicting proliferative T cells (CD8+ T cells also positive for Ki-67 proliferation marker positive), indicating immune activation following administration of the CAR construct. FIG. 4C shows dot plot (left) and bar graph (right) depicting higher number of cells with cytolytic activity as indicated by higher positive staining for marker Granzyme B by flow cytometry.

FIGS. 5A-5E depict expression of an anti-TROP2 CFP in myeloid cells from isolated whole blood following intravenous infusion of mRNA encoding anti-TROP2 CFP in a test composition (Test composition) in a TROP2+ HCC-1954 subcutaneous xenograft model in NCG Mice. FIG. 5A depicts the total Ly6C+ cells; FIG. 5B depicts Ly6C+ CD11b+ cells; FIG. 5C depicts Ly6C+ CD11c+ cells; and FIG. 5D depicts Ly6G+ cells. FIG. 5E is an experiment showing expression of the CAR in myeloid cell types. parent=total cells isolated having the marker denoted in the figure (e.g., CD11b+Ly6C+).

FIG. 6 depicts mean tumor volumes following administration of the mRNA-LNP compositions in a TROP2+ HCC-1954 subcutaneous xenograft model in NSG mice.

FIG. 7 shows an experiment demonstrating anti-tumor activity in of the anti-TROP2 CAR mRNA-LNP in suppression of tumor growth.

FIG. 8 depicts the frequency of anti-TROP2 CFPs in monocytes after intravenous infusion of the compositions in Cynomolgus monkeys.

FIG. 9 depicts tumor killing (left) and cytokine production in PBMCs transfected with the anti-TROP2-CD89 construct.

FIG. 10A depicts a schematic diagram of the first-in-human clinical study design for anti-TROP2-CD89-CAR LNP formulation in treatment of epithelial cancers.

FIG. 10B depicts an updated schematic diagram of the first-in-human clinical study design for anti-TROP2-CD89-CAR LNP formulation in treatment of epithelial cancers.

FIG. 11 shows a flowchart for the FIH trial conduct using BOIN design.

### DETAILED DESCRIPTION

T cells therapies have revolutionized cancer treatment for many patients. However, for the majority of patients with advanced solid tumors, sustained clinical benefit has not been achieved. Unlike T cells, myeloid cells readily accumulate in tumors, in some cases contributing up to 50% of the tumor mass. Myeloid cells can be specifically engineered to become highly effective anti-tumor cells, referred to as Activate, Target, Attack & Kill (ATAK) cells, that specifically target, phagocytize and lyse tumor cells, and orchestrate an immune activation in vivo against the tumor cells.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, is meant to encompass variations of +/−30% or less, +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

An “agent” can refer to any cell, small molecule chemical compound, antibody or fragment thereof, nucleic acid molecule, or polypeptide.

An “alteration” or “change” can refer to an increase or decrease. For example, an alteration can be an increase or decrease of 1%, 2%, 3%, 4%, 5%, 10%, 20%, 30%, or by 40%, 50%, 60%, or even by as much as 70%, 75%, 80%, 90%, or 100%. For example, an alteration can be an increase or decrease of 1-fold, 2-fold, 3-fold, 4-fold, 5-fold, 10-fold, 20-fold, 30-fold, or by 40-fold, 50-fold, 60-fold, or even by as much as 70-fold, 75-fold, 80-fold, 90-fold, or 100-fold.

An “antigen presenting cell” or “APC” as used herein includes professional antigen presenting cells (e.g., B lymphocytes, macrophages, monocytes, dendritic cells, Langerhans cells), as well as other antigen presenting cells (e.g., keratinocytes, endothelial cells, astrocytes, fibroblasts, oligodendrocytes, thymic epithelial cells, thyroid epithelial cells, glial cells (brain), pancreatic beta cells, and vascular endothelial cells). An APC can express Major Histocompatibility complex (MHC) molecules and can display antigens complexed with MHC on its surface which can be recognized by T cells and trigger T cell activation and an immune response. Professional antigen-presenting cells, notably dendritic cells, play a key role in stimulating naive T cells. Nonprofessional antigen-presenting cells, such as fibroblasts, may also contribute to this process. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules.

A “biological sample” can refer to any tissue, cell, fluid, or other material derived from an organism.

In some embodiments, a drug, e.g. a medicinal product, a therapeutic substance, or a pharmaceutical product may be referred to by its drug substance (DS), which is the ingredient within the drug that causes the medicinal effect; it may also be understood as the active pharmaceutical ingredient (API) of a pharmaceutical composition, for example, an engineered mRNA encoding a chimeric antigen receptor (CAR) is the API of a pharmaceutical composition, the latter further comprises the LNP and or the solution (excipient) etc. The final composition that is administered may be referred to as a drug product (DP).

The term “epitope” can refer to any protein determinant, such as a sequence or structure or amino acid residues, capable of binding to an antibody or binding fragment thereof, a T cell receptor, and/or an antibody-like molecule. Epitopic determinants typically consist of chemically active surface groups of molecules such as amino acids or sugar side chains and generally have specific three dimensional structural characteristics as well as specific charge characteristics. A “T cell epitope” can refer to peptide or peptide-MHC complex recognized by a T cell receptor.

An engineered cell, such as an engineered myeloid cell, can refer to a cell that has at least one exogenous nucleic acid sequence in the cell, even if transiently expressed. Expressing an exogenous nucleic acid may be performed by various methods described elsewhere, and encompasses methods known in the art. The present disclosure relates to preparing and using engineered cells, for example, engineered myeloid cells, such as engineered phagocytic cells. The present disclosure relates to, inter alia, an engineered cell comprising an exogenous nucleic acid encoding, for example, a chimeric antigenic receptor (CAR), interchangeably called herein as a chimeric fusion protein (CFP).

An engineered polynucleotide is a polynucleotide that is not found naturally in an organism. It is generated using molecular biology techniques, using, for example, cutting and ligating pieces of polynucleotides (e.g., DNA) from segments of same or different genes or pieces of nucleic acids that are not originally present in the same orientation or organization as is present in the engineered polynucleotide. An engineered polynucleic acid may be a plasmid, a recombinant DNA, a synthesized DNA, synthesized RNA, in vitro transcribed RNA, circularized RNA etc.

The term “immune response” includes, but is not limited to, T cell mediated, NK cell mediated and/or B cell mediated immune responses. These responses may be influenced by modulation of T cell costimulation and NK cell costimulation. Exemplary immune responses include T cell responses, e.g., cytokine production, and cellular cytotoxicity. In addition, immune responses include immune responses that are indirectly affected by NK cell activation, B cell activation and/or T cell activation, e.g., antibody production (humoral responses) and activation of cytokine responsive cells, e.g., macrophages. Immune responses include adaptive immune responses. The adaptive immune system can react to foreign molecular structures, such as antigens of an intruding organism. Unlike the innate immune system, the adaptive immune system is highly specific to a pathogen. Adaptive immunity can also provide long-lasting protection. Adaptive immune reactions include humoral immune reactions and cell-mediated immune reactions. In humoral immune reactions, antibodies secreted by B cells into bodily fluids bind to pathogen-derived antigens leading to elimination of the pathogen through a variety of mechanisms, e.g. complement-mediated lysis. In cell-mediated immune reactions, T cells capable of destroying other cells are activated. For example, if proteins associated with a disease are present in a cell, they can be fragmented proteolytically to peptides within the cell. Specific cell proteins can then attach themselves to the antigen or a peptide formed in this manner, and transport them to the surface of the cell, where they can be presented to molecular defense mechanisms, such as T cells. Cytotoxic T cells can recognize these antigens and kill cells that harbor these antigens.

A “ligand” can refer to a molecule which is capable of binding or forming a complex with another molecule, such as a receptor. A ligand can include, but is not limited to, a protein, a glycoprotein, a carbohydrate, a lipoprotein, a hormone, a fatty acid, a phospholipid, or any component that binds to a receptor. In some embodiments, a receptor has a specific ligand. In some embodiments, a receptor may have promiscuous binding to a ligand, in which case it can bind to several ligands that share at least a similarity in structural configuration, charge distribution or any other physicochemical characteristic. A ligand may be a biomolecule. A ligand may be an abiotic material. For example, a ligand may be a negative charged particle that is a ligand for scavenger receptor MARCO. For example, a ligand may be TiO₂, which is a ligand for the scavenger receptor SRA1. In the context of a CFP described herein, the extracellular binding domain may bind to a ligand, which is a also designated as a target of the binding domain. In some embodiments, the target is an antigen expressed on a diseased cell, such as a cancer cell, which in this case is a target cell, in the sense that the target cell expresses on its cell surface a target antigen to which the extracellular antigen binding domain of the CFP binds. Anti-(target) binding domain or anti-(target) binding extracellular domain or anti-(target) CFP are often interchangeably used with terms such as (target) binding domain or (target) binding extracellular domain or (target) CFP respectively in the disclosure. For example, HER2 expressed on cancer cells is an antigen (ligand) to which the anti-HER2 binding extracellular domain of a CFP binds; or alternatively stated as, a HER2-binding extracellular domain of a CFP binds.

The term “major histocompatibility complex (MHC)”, “MHC molecule”, or “MHC protein” may refer to a protein capable of binding an antigenic peptide and present the antigenic peptide to T lymphocytes. Such antigenic peptides can represent T cell epitopes. The human MHC is also called the HLA complex. Thus, the terms “human leukocyte antigen (HLA)”, “HLA molecule” or “HLA protein” are used interchangeably with the terms “major histocompatibility complex (MHC)”, “MHC molecule”, and “MHC protein”. HLA proteins can be classified as HLA class I or HLA class II. The structures of the proteins of the two HLA classes are very similar; however, they have very different functions. Class I HLA proteins are present on the surface of almost all cells of the body, including most tumor cells. Class I HLA proteins are loaded with antigens that usually originate from endogenous proteins or from pathogens present inside cells, and are then presented to naïve or cytotoxic T-lymphocytes (CTLs). HLA class II proteins are present on antigen presenting cells (APCs), including but not limited to dendritic cells, B cells, and macrophages. They mainly present peptides which are processed from external antigen sources, e.g. outside of cells, to helper T cells.

In the HLA class II system, phagocytes such as macrophages and immature dendritic cells can take up entities by phagocytosis into phagosomes—though B cells exhibit the more general endocytosis into endosomes—which fuse with lysosomes whose acidic enzymes cleave the uptaken protein into many different peptides. Autophagy is another source of HLA class II peptides. The most studied subclass II HLA genes are: HLA-DPA1, HLA-DPB1, HLA-DQA1, HLA-DQB1, HLA-DRA, and HLA-DRB1.

Presentation of peptides by HLA class II molecules to CD4+ helper T cells can lead to immune responses to foreign antigens. Once activated, CD4+ T cells can promote B cell differentiation and antibody production, as well as CD8+ T cell (CTL) responses. CD4+ T cells can also secrete cytokines and chemokines that activate and induce differentiation of other immune cells. HLA class II molecules are typically heterodimers of α- and β-chains that interact to form a peptide-binding groove that is more open than class I peptide-binding grooves.

HLA alleles are typically expressed in codominant fashion. For example, each person carries 2 alleles of each of the 3 class I genes, (HLA-A, HLA-B and HLA-C) and so can express six different types of class II HLA. In the class II HLA locus, each person inherits a pair of HLA-DP genes (DPA1 and DPB1, which encode α and β chains), HLA-DQ (DQA1 and DQB1, for α and β chains), one gene HLA-DRα (DRA1), and one or more genes HLA-DRβ (DRB1 and DRB3, -4 or -5). HLA-DRB1, for example, has more than nearly 400 known alleles. That means that one heterozygous individual can inherit six or eight functioning class II HLA alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the class I HLA alleles are HLA-A*02:01, HLA-B*14:02, HLA-A*23:01, HLA-E*01:01 (non-classical). In some embodiments, class II HLA alleles are HLA-DRB*01:01, HLA-DRB*01:02, HLA-DRB*11:01, HLA-DRB*15:01, and HLA-DRB*07:01.

A “myeloid cell” can refer broadly to cells of the myeloid lineage of the hematopoietic cell system, and can exclude, for example, the lymphocytic lineage. Myeloid cells comprise, for example, cells of the granulocyte lineage and monocyte lineages. Myeloid cells are a major cellular compartment of the immune system comprising monocytes, dendritic cells, tissue macrophages, and granulocytes. Models of cellular ontogeny, activation, differentiation, and tissue-specific functions of myeloid cells have been revisited during the last years with surprising results. However, their enormous plasticity and heterogeneity, during both homeostasis and disease, are far from understood. Although myeloid cells have many functions, including phagocytosis and their ability to activate T cells, harnessing these functions for therapeutic uses has remained elusive. Newer avenues are therefore sought for using other cell types towards development of improved therapeutics, including but not limited to T cell malignancies.

Myeloid cells are differentiated from common progenitors derived from the hematopoietic stem cells in the bone marrow. Commitment to myeloid cell lineages may be governed by activation of distinct transcription factors, and accordingly myeloid cells may be characterized as cells having a level of plasticity, which may be described as the ability to further differentiate into terminal cell types based on extracellular and intracellular stimuli. Myeloid cells can be rapidly recruited into local tissues via various chemokine receptors on their surface. Myeloid cells are responsive to various cytokines and chemokines.

A myeloid cell, for example, may be a cell that originates in the bone marrow from a hematopoietic stem cell under the influence of one or more cytokines and chemokines, such as G-CSF, GM-CSF, Flt3L, CCL2, VEGF and S100A8/9. In some embodiments, the myeloid cell is a precursor cell. In some embodiments, the myeloid cell may be a cell having characteristics of a common myeloid progenitor, or a granulocyte progenitor, a myeloblast cell, or a monocyte-dendritic cell progenitor or a combination thereof. A myeloid can include a granulocyte or a monocyte or a precursor cell thereof. A myeloid can include an immature granulocyte, an immature monocyte, an immature macrophage, an immature neutrophil, and an immature dendritic cell. A myeloid can include a monocyte or a pre-monocytic cell or a monocyte precursor. In some cases, a myeloid cell as used herein may refer to a monocyte having an M0 phenotype, an M1 phenotype or an M2 phenotype. A myeloid can include a dendritic cell (DC), a mature DC, a monocyte derived DC, a plasmacytoid DC, a pre-dendritic cell, or a precursor of a DC. A myeloid can include a neutrophil, which may be a mature neutrophil, a neutrophil precursor, or a polymorphonucleocyte (PMN). A myeloid can include a macrophage, a monocyte-derived macrophage, a tissue macrophage, a macrophage of an M0, an M1 or an M2 phenotype. A monocyte or a macrophage exhibit polarization. “Polarization” as used herein may refer to a process by which macrophages exhibit distinct functional phenotypes in response to specific microenvironmental stimuli and signals, often referred to as physiological states. In some cases, macrophages can pass from one polarization state to another. For example, macrophages can be polarized into classically activated (M1) and alternatively activated (M2) macrophages. M2 macrophages are divided into M2a, M2b, M2c, and M2d subcategories. These macrophages differ in their cell surface markers, secreted cytokines and biological functions. M1 macrophages are typically characterized by phenotypes in which the cells express TLR-2, TLR-4, CD80, CD86, iNOS, and MHC-II on the surface. These cells release various cytokines and chemokines e.g., TNF-α, IL-1α, IL-1β, IL-6, IL-12, CXCL9, and CXCL10, and typically exhibit activation of transcription factors, such as NF-κB, STAT1, STAT5, IRF3, and IRF5 that regulate the expression of M1 genes. It is believed that NF-κB and STAT1 are the two major pathways involved in M1 macrophage polarization. The M1 phenotype is associated with microbicidal and tumoricidal functions of macrophages, exhibiting high phagocytic and inflammatory function. On the other hand, tumor associated macrophages subject to immunosuppressive environment become generally more M2 polarized. A myeloid can include a tumor infiltrating monocyte (TIM). A myeloid can include a tumor associated monocyte (TAM). A myeloid can include a myeloid derived suppressor cell (MDSC). A myeloid can include a tissue resident macrophage. A myeloid can include a tumor associated DC (TADC). Accordingly, a myeloid cell may express one or more cell surface markers, for example, CD11b, CD14, CD15, CD16, CD38, CCR5, CD66, Lox-1, CD11c, CD64, CD68, CD163, CCR2, CCR5, HLA-DR, CD1c, CD83, CD141, CD209, MHC-II, CD123, CD303, CD304, a SIGLEC family protein and a CLEC family protein. In some cases, a myeloid cell may be characterized by a high or a low expression of one or more of cell surface markers, for example, CD11b, CD14, CD15, CD16, CD66, Lox-1, CD11c, CD64, CD68, CD163, CCR2, CCR5, HLA-DR, CD1c, CD83, CD141, CD209, MHC-II, CD123, CD303, CD304 or a combination thereof. In one embodiment, activating the M1 polarization of macrophages are desirable using the methods described herein.

“Phagocytosis” is used interchangeably with “engulfment” and can refer to a process by which a cell engulfs a particle, such as a cancer cell or an infected cell. This process can give rise to an internal compartment (phagosome) containing the particle. This process can be used to ingest and or remove a particle, such as a cancer cell or an infected cell from the body. A phagocytic receptor may be involved in the process of phagocytosis. The process of phagocytosis can be closely coupled with an immune response and antigen presentation. The processing of exogenous antigens follows their uptake into professional antigen presenting cells by some type of endocytic event. Phagocytosis can also facilitate antigen presentation. For example, antigens from phagocytosed cells or pathogens, including cancer antigens, can be processed and presented on the cell surface of APCs.

A “polypeptide” can refer to a molecule containing amino acids linked together via a peptide bond, such as a glycoprotein, a lipoprotein, a cellular protein or a membrane protein. A polypeptide may comprise one or more subunits of a protein. A polypeptide may be encoded by a recombinant nucleic acid. In some embodiments, polypeptide may comprise more than one peptide sequence in a single amino acid chain, which may be separated by a spacer, a linker or peptide cleavage sequence. A polypeptide may be a fused polypeptide. A polypeptide may comprise one or more domains, modules or moieties.

In some embodiments, a drug that is being investigated may be evaluated by pharmacokinetic (PK) and pharmacodynamic (PD) studies (pharmacokinetics). Such studies may usually include investigating how a drug enters and processes through the metabolic system and exits, including but not limited to absorption by different tissues, accumulation in tissue or organs, and excretion, rate of passage through a tissue or organ or the organismic system.

A “receptor” can refer to a chemical structure composed of a polypeptide, which transduces a signal, such as a polypeptide that transduces an extracellular signal to a cell. A receptor can serve to transmit information in a cell, a cell formation or an organism. A receptor comprises at least one receptor unit and can contain two or more receptor units, where each receptor unit comprises a protein molecule, e.g., a glycoprotein molecule. A receptor can contain a structure that binds to a ligand and can form a complex with the ligand. Signaling information can be transmitted by a conformational change of the receptor following binding with the ligand on the surface of a cell.

The term “antibody” may refer to a class of proteins that are generally known as immunoglobulins, including, but not limited to IgG1, IgG2, IgG3, and IgG4), IgA (including IgA1 and IgA2), IgD, IgE, IgM, and IgY, The term “antibody” includes, but is not limited to, full length antibodies, single-chain antibodies, single domain antibodies (sdAb) and antigen-binding fragments thereof. Antigen-binding antibody fragments include, but are not limited to, Fab, Fab′ and F(ab′)2, Fd (consisting of V_{H} and C_{H}1), single-chain variable fragment (scFv), single-chain antibodies, disulfide-linked variable fragment (dsF_{V}) and fragments comprising a V_{L} and/or a V_{H} domain. Antibodies can be from any animal origin. Antigen-binding antibody fragments, including single-chain antibodies, can comprise variable region(s) alone or in combination with tone or more of a hinge region, a CH1 domain, a CH2 domain, and a CH3 domain. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex.

The term “recombinant nucleic acid” refers a nucleic acid prepared, expressed, created or isolated by recombinant means. A recombinant nucleic acid can contain a nucleotide sequence that is not naturally occurring. A recombinant nucleic acid may be synthesized in the laboratory. A recombinant nucleic acid may be prepared by using recombinant DNA technology, for example, enzymatic modification of DNA, such as enzymatic restriction digestion, ligation, and DNA cloning. A recombinant nucleic acid can be DNA, RNA, analogues thereof, or a combination thereof. A recombinant DNA may be transcribed ex vivo or in vitro, such as to generate a messenger RNA (mRNA). A recombinant mRNA may be isolated, purified and used to transfect a cell. A recombinant nucleic acid may encode a protein or a polypeptide. Throughout the specification, nucleic acid sequences are described which may comprise deoxyribonucleotides (DNA), ribonucleotides (RNA), or in some embodiments, modified deoxyribonucleotides, or modified ribonucleotides. For example, a modified nucleotide may be a 5-hydroxymethyleytosine (5hmC), a 5-formylacytosine (5fC), a 7-methylguanosine, a pseudouridine, a dihydrouridine etc. One of skill in the art can determine an RNA sequence, e.g., an mRNA sequence from a given polynucleotide sequence without difficulty. Sequences may be codon optimized.

The process of introducing or incorporating a nucleic acid into a cell can be via transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the introduction of small molecules such as DNA, RNA, or antibodies into eukaryotic cells. Transfection may also refer to the introduction of bacteriophage into bacterial cells. ‘Transduction’ is mostly used to describe the introduction of recombinant viral vector particles into target cells, while ‘infection’ may refer to natural infections of humans or animals with wild-type viruses.

The term “vector”, can refer to a nucleic acid molecule capable of autonomous replication in a host cell, and which allow for cloning of nucleic acid molecules. As known to those skilled in the art, a vector includes, but is not limited to, a plasmid, cosmid, phagemid, viral vectors, phage vectors, yeast vectors, mammalian vectors and the like. For example, a vector for exogenous gene transformation may be a plasmid. In certain embodiments, a vector comprises a nucleic acid sequence containing an origin of replication and other elements necessary for replication and/or maintenance of the nucleic acid sequence in a host cell. In some embodiments, a vector or a plasmid provided herein is an expression vector. Expression vectors are capable of directing the expression of genes and/or nucleic acid sequence to which they are operatively linked. In some embodiments, an expression vector or plasmid is in the form of circular double stranded DNA molecules. A vector or plasmid may or may not be integrated into the genome of a host cell. In some embodiments, nucleic acid sequences of a plasmid are not integrated in a genome or chromosome of the host cell after introduction. For example, the plasmid may comprise elements for transient expression or stable expression of the nucleic acid sequences, e.g. genes or open reading frames harbored by the plasmid, in a host cell. In some embodiments, a vector is a transient expression vector. In some embodiments, a vector is a stably expressed vector that replicates autonomously in a host cell. In some embodiments, nucleic acid sequences of a plasmid are integrated into a genome or chromosome of a host cell upon introduction into the host cell. Expression vectors that can be used in the methods as disclosed herein include, but are not limited to, plasmids, episomes, bacterial artificial chromosomes, yeast artificial chromosomes, bacteriophages or viral vectors. A vector can be a DNA or RNA vector. In some embodiments, a vector provide herein is a RNA vector that is capable of integrating into a host cell's genome upon introduction into the host cell (e.g., via reverse transcription), for example, a retroviral vector or a lentiviral vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

In some embodiments, nucleic acid may be delivered into a living system in the form of nanoparticles. Nucleic acid sequences disclosed herein may be delivered in vivo via suitable nanoparticles, e.g., liposomes, lipid nanoparticles, or polymeric nanoparticles. A lipid nanoparticle may comprise a polar lipid. In some embodiments, the lipid nanoparticle comprises a cationic lipid. In some embodiments, the lipid nanoparticle comprises a cationic lipid and a non-cationic lipid. In some embodiments, the lipid nanoparticle comprises a neutral lipid. In some embodiments, the lipid nanoparticle comprises a PEGylated lipid.

Alternatively, in some embodiments, the nucleic acid can be electroporated in a living cell ex vivo for preparation of a cellular therapy, wherein the cell is a myeloid cell.

In some embodiments, a “spacer” or a “linker” as used herein in reference to a fusion protein may refer to a peptide sequence that joins two other peptide sequences of the fusion protein. In some embodiments, a linker or spacer has no specific biological activity other than to join or to preserve some minimum and/or maximum distance or confers some other spatial relationship between two or more peptide sequences of the fusion protein. In some embodiments, the constituent amino acids of a spacer can be selected to influence some property of the fusion protein molecule such as the folding, flexibility, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. In some embodiments, a linker is used to separate two or more polypeptides, e.g. two antigenic peptides by a distance sufficient to ensure that each antigenic peptide properly folds. Exemplary peptide linker sequences adopt a flexible extended conformation and do not exhibit a propensity for developing an ordered secondary structure. Amino acids in flexible linker protein region may include Gly, Asn and Ser, or any permutation of amino acid sequences containing Gly, Asn and Ser. Other near neutral amino acids, such as Thr and Ala, also can be used in the linker sequence.

In some aspects, the instant disclosure provides a method and compositions for treating a disease or a disorder, wherein treating and grammatical variants of the same may refer to administering a composition, e.g., a pharmaceutical composition for reducing, preventing, or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” may include the concepts of “alleviating”, which can refer to lessening the frequency of occurrence or recurrence, or the severity, of at least one symptom or an ill effect related to the disease and/or a side effect associated with therapy. Treating may also encompasses the concept of “managing” which may refer to reducing the severity of a disease or disorder in a patient, e.g., extending the life or prolonging the survivability of a patient with the disease, or delaying its recurrence, e.g., extending the period of progression free survival in a disease such as cancer in a patient who is or has suffered from the disease. It is appreciated that, although not precluded, treating a disorder or condition does not require that the disorder, condition, or symptoms associated therewith be completely eliminated. In some embodiments, “prevent”, “preventing”, “prevention” and their grammatical equivalents as used herein, can refer to avoiding or delaying the onset of symptoms associated with a disease or condition in a subject that has not developed such symptoms at the time the administering of an agent or compound commences. In certain embodiments, treating a subject or a patient as described herein comprises administering a therapeutic composition, such as a drug, a metabolite, a preventive component, a nucleic acid, a peptide, or a protein that encodes or otherwise forms a drug, a metabolite or a preventive component. In some embodiments, treating comprises administering a cell or a population of cells to a subject in need thereof. In some embodiments, treating comprises administering to the subject one or more of engineered cells described herein, e.g. one or more engineered myeloid cells, such as phagocytic cells. Treating comprises treating a disease or a condition or a syndrome, which may be a pathological disease, condition or syndrome, or a latent disease, condition or syndrome. In some embodiments, treating may comprise administering a therapeutic, such as a vaccine. In some embodiments, the engineered phagocytic cell is administered to a patient or a subject. In some embodiments, a cell administered to a human subject results in reduced immunogenicity. For example, an engineered phagocytic cell may lead to no or reduced graft versus host disease (GVHD) or fratricide effect. In some embodiments, an engineered cell administered to a human subject is immunocompatible to the subject (i.e. having a matching HLA subtype that is naturally expressed in the subject). Subject specific HLA alleles or HLA genotype of a subject can be determined by any method known in the art. In exemplary embodiments, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

In some embodiments, “fragment” can refer to a portion of a protein or nucleic acid. In some embodiments, a fragment retains at least 50%, 75%, or 80%, or 90%, 95%, or even 99% of the biological activity of a reference protein or nucleic acid.

The terms “isolated,” “purified”, “biologically pure” and their grammatical equivalents may usually refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

In some embodiments, “neoplasia” or “cancer” may refer to any disease that is caused by or results in inappropriately high levels of cell division, inappropriately low levels of apoptosis, or both. Glioblastoma is one non-limiting example of a neoplasia or cancer. The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell.

In some embodiments, “vaccine” may be understood as meaning a composition for generating immunity for the prophylaxis and/or treatment of diseases (e.g., neoplasia/tumor/infectious agents/autoimmune diseases). Accordingly, vaccines as used herein are medicaments which comprise recombinant nucleic acids, or cells comprising and expressing a recombinant nucleic acid and are intended to be used in humans or animals for generating specific defense and protective substance by vaccination. A “vaccine composition” can include a pharmaceutically acceptable excipient, carrier or diluent. Aspects of the present disclosure relate to use of the technology in preparing a phagocytic cell-based vaccine.

In some embodiments, “pharmaceutically acceptable” may refer to approved or approvable by a regulatory agency of the Federal or a state government or listed in the U.S. Pharmacopeia or other generally recognized pharmacopeia for use in animals, including humans. In some embodiments, “pharmaceutically acceptable excipient, carrier or diluent” may refer to an excipient, carrier or diluent that can be administered to a subject, together with an agent, and which does not destroy the pharmacological activity thereof and is nontoxic when administered in doses sufficient to deliver a therapeutic amount of the agent.

Nucleic acid molecules useful in the methods of the disclosure include, but are not limited to, any nucleic acid molecule with activity or that encodes a polypeptide. Polynucleotides having substantial identity to an endogenous sequence are typically capable of hybridizing with at least one strand of a double-stranded nucleic acid molecule. In some embodiments, “hybridize” may refer to when nucleic acid molecules pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions can ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an exemplary embodiment, hybridization can occur at 30° C. in 750 mM NaCl, 75 mM trisodium citrate, and 1% SDS. In another exemplary embodiment, hybridization can occur at 37° C. in 500 mM NaCl, 50 mM trisodium citrate, 1% SDS, 35% formamide, and 100 μg/ml denatured salmon sperm DNA (ssDNA). In another exemplary embodiment, hybridization can occur at 42° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25° C., of at least about 42° C., or at least about 68° C. In exemplary embodiments, wash steps can occur at 25° C. in 30 mM NaCl, 3 mM trisodium citrate, and 0.1% SDS. In other exemplary embodiments, wash steps can occur at 42° C. in 15 mM NaCl, 1.5 mM trisodium citrate, and 0.1% SDS. In another exemplary embodiment, wash steps can occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In some embodiments, “substantially identical” may refer to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence can be at least 60%, 80% or 85%, 90%, 95%, 96%, 97%, 98%, or even 99% or more identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. In some embodiments, a “reference” is a standard of comparison. It may be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment to a reference sequence and determination of homologous residues.

The term “subject” or “patient” may refer to an organism, such as an animal (e.g., a human) which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

In some embodiments, a therapeutic effect may be noted or may be expected, which may refer to an extent of relief of one or more symptoms associated with a disorder (e.g., a neoplasia, tumor, or infection by an infectious agent or an autoimmune disease) or an effect that is in some way measurable in relation to a disease-associated pathology, for example, a viral titer in a biological sample from a subject being treated with an antiviral therapeutic. In one embodiment, a therapeutic effect may indicate a reduction of a symptom of the disease following an administration of the therapy in the subject, e.g., a 5%, 10%, 20%, 30% etc. reduction in tumor mass following administration of the therapeutic composition. In another embodiment, a therapeutic effect can relate to partial or complete remission of one or more symptoms, or amelioration of the disease. Therapeutically effective amount as used herein may refer to an amount of an agent, e.g., a pharmaceutical composition which, at a time following administration is effective, upon single or multiple repeats (e.g., doses) of the administration, show an indication of reduction or amelioration of a symptom or a parameter associated with the disease for which the therapeutic is administered. For example, a therapeutically effective amount may be an amount that is associated with prolongation of survivability of a patient with such a disorder. In some embodiment, a therapeutically effective amount of a reducing one or more signs or symptoms of the disorder, preventing or delaying, and the like beyond that expected in the absence of such treatment. Therapeutically effective amount is typically intended to qualify the amount required to achieve a therapeutic effect. Such therapeutic effect may be closely associated with the dose (amount of the therapeutic), the schedule or the therapeutic regimen, and may differ from one subject to another. A schedule or a therapeutic regimen may be understood as the frequency of administering a therapeutic at a certain interval of time, such as once daily, once a week, one every two weeks and so on, and in combination with total duration of the therapeutic administration. Hence, an example of a therapeutic regimen may comprise administering a therapeutic (e.g. a pharmaceutical composition), at a certain dose (e.g., amount), at an interval of once every seven days for a duration (e.g., total period) of six months, during or upon completion of which, a therapeutic effect may be noted or expected. For example, a therapeutic effect of an epithelial cancer may be a reduction of the cancerous lesion on the epithelial tissue. A physician or veterinarian with specialized skill may determine and prescribe the “therapeutically effective amount” (e.g., ED50) of the pharmaceutical composition required.

Provided herein are engineered myeloid cells (including, but not limited to, neutrophils, monocytes, myeloid dendritic cells (mDCs), mast cells and macrophages), designed to specifically bind a target antigen. The target antigen may be expressed only on a target cell, such as an infected cell, a damaged cell, a malignant cell, a leukemia cell or a tumor cell. The engineered myeloid cells can attack and kill target cells directly (e.g., by phagocytosis) and/or indirectly (e.g., by activating T cells). In some embodiments, the target cell is a cancer cell.

While cancer is one exemplary embodiment described in detail in the instant disclosure, the methods and technologies described herein are contemplated to be useful in targeting an infected or otherwise diseased cell inside the body. Similarly, therapeutic and vaccine compositions using the engineered cells are described herein.

Myeloid effector cells may be generated from the isolated myeloid cells from a human biological sample and modified ex vivo to prepare cells of therapeutic interest using methods to engineer such cells and such that the modifications do not alter the plasticity of these cells. Monocytic lineage cells are phagocytic and are efficient antigen presenter cells. In one aspect, the present invention stems from an important finding that engineered myeloid cells can be a highly efficient therapeutic modality in treating a number of diseases including cancer. Myeloid cells may be engineered to express a chimeric antigen receptor that enhances a myeloid cell's immune function in which the cells are highly phagocytic and can attack and kill a diseased cell or an infected cell in the body. The chimeric antigen receptor is a recombinant construct that is designed, and specifically modified as described herein, to be (a) highly target specific, specifically directed to bind to a target antigen, having an extracellular antigen binding domain, and (b) an intracellular domain that is highly specialized to activate a myeloid cell to attain an activate phagocytic cell phenotype. For example, highly specialized intracellular domains are designed to generate chimeric receptors that, upon activation by binding of the extracellular region of the receptor to the target, can generate signaling cues inside the cell that activate intracellular interferon signaling cascade, and transcription factors, namely, directs the activation of transcription factor IRFs (IFN regulatory factors). In addition, the methods and compositions described herein are also useful in gene therapy in which a recombinant nucleic acid encoding a chimeric antigen receptor is administered locally or systemically in a subject in need thereof, such that the recombinant nucleic acid is specifically expressed in a myeloid cell in vivo, and thereby generates activated myeloid cells having the therapeutic ability. In some embodiments, the nucleic acid is mRNA. In some embodiments the mRNA is delivered in an LNP.

Phagocytes are often designated as the natural sentinels of the immune system and form the first line of defense in the body. They engulf a pathogen, a pathogen infected cell, a foreign body, or a cancerous cell and remove it from the body. Most potential pathogens are rapidly neutralized by this system before they can cause, for example, a noticeable infection or a disease. This can involve receptor-mediated uptake through the clathrin-coated pit system, pinocytosis, particularly macropinocytosis as a consequence of membrane ruffling and phagocytosis. The phagocytes therefore can be activated by a variety of non-self (and self) elements and exhibit a level of plasticity in recognition of their “targets”. Most phagocytes express scavenger receptors on their surface which are pattern recognition molecules and can bind to a wide range of foreign particles as well as dead cell, debris and unwanted particles within the body. In one aspect, recombinant nucleic acids encoding chimeric antigen receptors (CAR) may be expressed in the cells. The CARs may be variously designed to attack specific tumor cells, and myeloid effector cells expressing CARs can be activated to phagocytose and kill tumor cells. The CARs may be designed to generate phagocytic receptors that are activated specifically in response to the target engagement, and the phagocytic potential of a macrophage is enhanced by specifically engineered intracellular domains of the receptor. The CAR platform for myeloid cells as described herein is designed such that no tonic signaling is detected in the myeloid cells at the time of administering in the body, or any time before the myeloid cell engages with its target via the CAR. This is often tested ex vivo. At the same time, the myeloid cells expressing the CAR can be further differentiated into M0, M1 or M2 phenotypes in presence of the suitable stimulus, and retain the cellular plasticity to do so at least at the time of administration. In addition, CAR-expressing myeloid effector cells can migrate to lymph nodes and cross-present antigens to naïve T cells in the lymph node thereby activating the adaptive response.

In some embodiments, disclosed herein are compositions and methods for generating myeloid cells that are isolated from a biological sample and engineered ex vivo to express a recombinant protein, and formulated into a pharmaceutical composition, such that the myeloid cells of the composition are “effector” myeloid cells efficient in induction of immune activation in vivo. In some embodiments, the myeloid cells of the composition are termed ‘ATAK’ myeloid cells where the cells are myeloid efficient in attacking and destroying target cells. The ATAK myeloid cells disclosed herein is an engineered myeloid cell that expresses a recombinant protein, e.g., a chimeric receptor, e.g., a chimeric antigen receptor, comprising at least one intracellular signaling domain that is derived from an interferon inducing protein in an immune cell. In some embodiments, the methods and compositions described herein are directed to render an engineered myeloid cell to exhibit the effector phenotype. In some embodiments, the engineered myeloid cells, e.g., monocytes are M0 or M1 phenotype monocytes, and the activation of the chimeric antigen receptor expressed in the myeloid cell renders the cells to exhibit the M1 phenotype. The M1 phenotype exhibited by the engineered cell renders the cell to be highly tumoricidal when designed to be targeted to a tumor cell.

Provided herein are compositions and methods for treating diseases or conditions, such as cancer. The compositions and methods provided herein utilize human myeloid cells, including, but not limited to, neutrophils, monocytes, myeloid dendritic cells (mDCs), mast cells and macrophages, to target diseased cells, such as cancer cells. The compositions and methods provided herein can be used to eliminate diseased cells, such as cancer cells and or diseased tissue, by a variety of mechanisms, including T cell activation and recruitment, effector immune cell activation (e.g., CD8 T cell and NK cell activation), antigen cross presentation, enhanced inflammatory responses, reduction of regulatory T cells and phagocytosis. For example, the myeloid cells can be used to sustain immunological responses against cancer cells.

Applicants previously described compositions comprising a recombinant nucleic acid encoding a chimeric fusion protein (CFP), such as a phagocytic receptor (PR) fusion protein (PFP), a scavenger receptor (SR) fusion protein (SFP), an integrin receptor (IR) fusion protein (IFP) or a caspase-recruiting receptor (caspase-CAR) fusion protein. A CFP encoded by the recombinant nucleic acid can comprise an extracellular domain (ECD) comprising an antigen binding domain that binds to an antigen of a target cell. The extracellular domain can be fused to a hinge domain or an extracellular domain derived from a receptor, such as CD2, CD8, CD28, CD68, a phagocytic receptor, a scavenger receptor or an integrin receptor. The CFP encoded by the recombinant nucleic acid can further comprise a transmembrane domain, such as a transmembrane domain derived from CD2, CD8, CD28, CD68, a phagocytic receptor, a scavenger receptor or an integrin receptor. In some embodiments, a CFP encoded by the recombinant nucleic acid further comprises an intracellular domain comprising an intracellular signaling domain, such as an intracellular signaling domain derived from a phagocytic receptor, a scavenger receptor or an integrin receptor. For example, the intracellular domain can comprise one or more intracellular signaling domains derived from a phagocytic receptor, a scavenger receptor or an integrin receptor. For example, the intracellular domain can comprise one or more intracellular signaling domains that promote phagocytic activity, inflammatory response, nitric oxide production, integrin activation, enhanced effector cell migration (e.g., via chemokine receptor expression), antigen presentation, and/or enhanced cross presentation. In some embodiments, the CFP is a phagocytic receptor fusion protein (PFP). In some embodiments, the CFP is a phagocytic scavenger receptor fusion protein (PFP). In some embodiments, the CFP is an integrin receptor fusion protein (IFP). In some embodiments, the CFP is an inflammatory receptor fusion protein. In some embodiments, a CFP encoded by the recombinant nucleic acid further comprises an intracellular domain comprising a recruitment domain. For example, the intracellular domain can comprise one or more PI3K recruitment domains, caspase recruitment domains or caspase activation and recruitment domains (CARDs).

Provided herein are improved immunogenic CAR compositions, for example, recombinant nucleic acid encoding a chimeric fusion protein (CFP, interchangeably termed chimeric antigen receptor, CAR) comprising an intracellular domain that activates interferon response in a cell expressing the CAR. Provided herein are immunogenic CFPs that comprise at least one intracellular domain comprising a pLxIS motif. The recombinant nucleic acid may be DNA or RNA. The recombinant nucleic acid encoding the CAR may be comprised in a vector. The recombinant CAR when expressed in a cell activates Type I interferon production in the cell. Such a cell is a mammalian cell, that is capable of Type 1 Interferon response. Such cell is an immune cell, e.g. a lymphocyte cell or a myeloid cell.

In some embodiments the recombinant nucleic acid encoding the chimeric receptor comprises a specific sequence therein that encodes a pro-inflammatory intracellular domain of the chimeric receptor. In some embodiments, the chimeric receptor protein described herein comprises an intracellular domain capable of activating an interferon response gene or a signaling cascade leading to induction of Type I interferon production in the cell that expresses the chimeric antigen receptor upon engagement with its target at the extracellular domain. In some embodiments, the chimeric receptor protein described herein comprises a domain from an innate immune pathway adaptor protein, e.g., Mitochondrial antiviral-signaling protein (MAVS), Stimulator of interferon genes (STING), Toll/IL-1R domain-containing adaptor inducing IFN (TRIF), and TLR adaptor interacting with endolysosomal SLC15A4 protein (TASL), or a portion thereof. In some embodiments, a domain or fragment of an innate immune pathway adaptor protein e.g., MAVS, STING, TRIF or TASL proteins may be incorporated by recombinant DNA technology in the intracellular domain of the CFP or CAR as described herein, wherein the domain or fragment comprises a pLxIS motif (in which p represents the hydrophilic residue, x represents any residue, and S represents the phosphorylation site), that is phosphorylated by TBK1 or IKKε and mediates the recruitment of IRF-3 to the signaling complexes.

In some embodiments, the chimeric receptor protein described herein comprises an intracellular domain capable of activating nuclear factor kappa B responsive gene or a signaling cascade leading to induction of NF-kappa B response in the cell that expresses the chimeric antigen receptor upon engagement with its target at the extracellular domain.

### Effector Myeloid Cells and Interferon Activation

Type I and Type II interferons (IFNs) play important roles in regulating immune responses during infections and cancer. Type I is represented by multiple subtypes including numerous IFNα family members, IFNβ, IFNδ, IFNε, IFNκ, IFNτ and IFNω, and all these utilize the same cell surface receptor, IFNαR, which is a heterodimer comprised of IFNαR1 and IFNαR2 proteins. Type II IFN is represented by IFNγ. These two IFN types bind to distinct cell surface receptors that are expressed by nearly all cells to trigger signal transduction events and elicit diverse cellular responses. Myeloid cells are key targets of interferons. During early immune responses to intracellular bacterial infections. Activated natural killer (NK) and T cells are the sources of IFNγ production. During early stages of infection, production of the cytokines interleukin (IL)-12 and IL-18 drives antigen-nonspecific IFNγ production by these lymphocyte populations. Antigen-specific CD4+ and CD8+ T cells also can produce IFNγ in response to these pathogens. There are a large number of individual type I IFNs, including ~20 IFNα proteins and a single IFNβ. Each of these type I IFNs signals to host cells by binding the conserved cell surface type I IFN receptor, IFNαR. Ligation of cell surface IFNαR induces expression of numerous antiviral immune stimulated gene (ISG) products and thus protects the host from certain viral infections (Sadler A J, Interferon-inducible antiviral effectors. (Review) Nat Rev Immunol. 2008 July; 8 (7): 559-68). However, responsiveness to type I IFNs also correlates dramatically with increased susceptibility to a number of intracellular bacterial infections (Rayamajhi M., et al., Antagonistic crosstalk between type I and II interferons and increased host susceptibility to bacterial infections. Virulence. 2010 September-October; 1(5):418-22), including Listeria monocytogenes, Mycobacterium tuberculosis, Fransicella tularensis, and others. IFNγ is secreted as a homodimer and acts on host cells by ligating cell surface receptors. Each IFNγ receptor is a heterodimer comprised of two type I integral membrane subunits, IFNγR1 and IFNγR2. Binding of an IFNγ homodimer to the cell causes the aggregation of two receptor complexes, such that there are two IFNγR1 subunits and two IFNγR2 subunits, as well as additional signaling components. While both subunits are required for signal transduction, the actual binding site for IFNγ is located on IFNγR1 (Kearney S. et al., Differential effects of type I and II interferons on myeloid cells and resistance to intracellular bacterial infections. Immunol Res. 2013 March; 55 (0): 187-200). When IFNγ interacts with an IFNγR1 subunit, it induces a conformational change that permits a closer association of the IFNγR1 and IFNγR2 subunits. These rearrangements in the receptor induce auto- and cross-phosphorylation of Janus-associated kinases (JAKs) that are constitutively associated with the receptor. IFNγR1 contains a binding motif for JAK1, and IFNγR2 contains a binding motif for JAK2. Phosphorylation of the JAK proteins stimulates their catalytic activity and they then phosphorylate a tyrosine residue (Y₄₄₀) at the C-terminus of IFNγR1, This phosphorylated tyrosine residue provides a docking site for the SH2 domain on the Signal Transducer and Activator of Transcription-1 (STAT-1) protein. Because each receptor complex contains two IFNγR1 subunits, two STAT-1 proteins are able to bind to the receptor. JAK1 and JAK2 remain receptor-associated and phosphorylate each recruited STAT-1 protein at tyrosine residue 701 (Y 701). This phosphorylation allows release of the STAT-1 monomers from the receptor and their formation of homodimers. STAT-1 homodimers translocate to the nucleus and bind Gamma-Activated Sequences (GAS) in the promoter DNA of IFN-stimulated genes (ISGs), resulting in their increased transcription. Type I IFNs signal through a canonical JAK/STAT pathway, similar to that activated by IFNγ. Ligand binding to the IFNαR initiates dimerization of the two receptor subunits and trans-phosphorylation of their associated TYK2 and JAK1 kinases. The kinases phosphorylate residues in the cytoplasmic tails of IFNαR1 and IFNαR2 to recruit STAT1 and STAT2 proteins via their SH2 domains. Docking of these STAT proteins to the receptor subunits allows their phosphorylation by the activated JAK proteins at Y₇₀₁ on STAT-1 and Y₆₉₀ on STAT-2. Phosphorylation of the STAT monomers releases them from their docking site, allowing them to dimerize and combine in a homodimeric or heterodimeric form with IRF9 to produce the transcription factor ISG factor 3 (ISGF3). ISGF3 translocates into the nucleus to identify ISGs and induces their transcription. ISGs induced by type I IFN signaling typically contain interferon stimulated response element (ISRE) or a gamma activated sequence (GAS) elements within their promoters, although there is a clear preference for genes containing an ISRE. Some examples of ISGs transcribed as a result of type I IFNs are ISRE containing genes ISG15, IP-10, IRF-7 and PKR [66], and GAS containing genes IRF-1, IRF-2, IRF-8 and IRF-9 (Kearney S. et al., Differential effects of type I and II interferons on myeloid cells and resistance to intracellular bacterial infections. Immunol Res. 2013 March; 55 (0): 187-200).

### TROP2 Binding Chimeric Receptors for Phagocytic Cell Activation

### Extracellular Antigen Binding Domain

Table 1A shows exemplary sequences of chimeric fusion protein domains and/or fragments thereof that are meant to be non-limiting for the disclosure. Underlines denote the CDR sequences in sequential order of CDR1, CDR2 and CDR3 for the respective heavy and light chains in accordance to the Kabat numbering system.

TABLE 1A
Exemplary Chimeric Fusion Protein Antigen Binding Domains

| SEQ | Antigen | |
|---|---|---|
| ID | Binding | |
| NO | Domain | Sequence |
| 1 | Anti- | QVQLQQSGSELKKPGASVKVSCKASGYTFTNYGMNWVKQAPGQGLKWM |
| | TROP2 | GWINTYTGEPTYTDDFKGRFAFSLDTSVSTAYLQISSLKADDTAVYFCARG |
| | VH | GFGSSYWYFDVWGQGSLVTVSS |
| | domain | HCDR1 sequence: NYGMN (Kabat) GYTFTNY (Chothia), |
| | | GYTFTNYG (IMGT) |
| | | HCDR2 sequence: WINTYTGEPTYTDDFKG (Kabat), NTYTGE |
| | | (Chothia), INTYTGEP (IMGT) |
| | | HCDR3 sequence: GGFGSSYWYFDV (Kabat), GGFGSSYWYFDV(Chothia) |
| | | ARGGFGSSYWYFDV IMGT) |
| 2 | Anti- | DIQLTQSPSSLSASVGDRVSITCKASQDVSIAVAWYQQKPGKAPKLLIYSAS |
| | TROP2 | YRYTGVPDRFSGSGSGTDFTLTISSLOPEDFAVYYCQQHYITPLTFGAGTKV |
| | VL | EIKR |
| | domain | LCDR1 sequence: KASQDVSIAVA (Kabat), KASQDVSIAVA (Chothia), |
| | | QDVSIA (IMGT) |
| | | LCDR2 sequence: SASYRYT (Kabat), SASYRYT (Chothia), |
| | | SAS (IMGT) |
| | | LCDR3 sequence: QQHYITPLT (Kabat), QQHYITPLT (Chothia), |
| | | QQHYITPLT (IMGT) |
| 3 | Anti- | QVQLQQSGSELKKPGASVKVSCKASGYTFTNYGMNWVKQAPGQGLKWM |
| | TROP2 | GWINTYTGEPTYTDDFKGRFAFSLDTSVSTAYLQISSLKADDTAVYFCARG |
| | binding | GFGSSYWYFDVWGQGSLVTVSSGGGGSGGGGSGGGGSDIQLTQSPSSLSA |
| | domain | SVGDRVSITCKASQDVSIAVAWYQQKPGKAPKLLIYSASYRYTGVPDRFSG |
| | (scFv) | SGSGTDFTLTISSLOPEDFAVYYCQQHYITPLTFGAGTKVEIKR |

### Transmembrane Domains

In some embodiments, the transmembrane domain and the antigen binding domain are operatively linked through a linker. In some embodiments, the transmembrane domain and the antigen binding domain are operatively linked through a linker such as a hinge region of CD80, IgG1 or IgG4.

In some embodiments, the extracellular domain comprises a multimerization scaffold.

In some embodiments, the transmembrane domain comprises a CD8 transmembrane domain. In some embodiments, the transmembrane domain comprises a CD28 transmembrane domain. In some embodiments, the transmembrane domain comprises a CD68 transmembrane domain. In some embodiments, the transmembrane domain comprises a CD2 transmembrane domain. In some embodiments, the transmembrane domain comprises an FcR transmembrane domain. In some embodiments, the transmembrane domain comprises an FcRγ transmembrane domain. In some embodiments, the transmembrane domain comprises an FcRα transmembrane domain. In some embodiments, the transmembrane domain comprises an FcRβ transmembrane domain. In some embodiments, the transmembrane domain comprises an FcRε transmembrane domain. In some embodiments, the transmembrane domain comprises a transmembrane domain from a syntaxin, such as syntaxin 3 or syntaxin 4 or syntaxin 5. In some embodiments, the transmembrane domain oligomerizes with a transmembrane domain of an endogenous receptor when the CFP is expressed in a cell. In some embodiments, the transmembrane domain oligomerizes with a transmembrane domain of an exogenous receptor when the CFP is expressed in a cell. In some embodiments, the transmembrane domain dimerizes with a transmembrane domain of an endogenous receptor when the CFP is expressed in a cell. In some embodiments, the transmembrane domain dimerizes with a transmembrane domain of an exogenous receptor when the CFP is expressed in a cell. In some embodiments, the transmembrane domain is derived from a protein that is different than the protein from which the intracellular signaling domain is derived. In some embodiments, the transmembrane domain is derived from a protein that is different than the protein from which the extracellular domain is derived. In some embodiments, the transmembrane domain comprises a transmembrane domain of a phagocytic receptor. In some embodiments, the transmembrane domain and the extracellular domain are derived from the same protein. In some embodiments, the transmembrane domain is derived from the same protein as the intracellular signaling domain. In some embodiments, the recombinant nucleic acid encodes a DAP12 recruitment domain. In some embodiments, the transmembrane domain comprises a transmembrane domain that oligomerizes with DAP12.

In some embodiments, the transmembrane domain is at least 12, 13, 14, 15, 16, 17, 18, 19, 20, 21, 22, 23, 24, 25, 26, 27, 28, 29, 30, 31 or 32 amino acids in length. In some embodiments, the transmembrane domain is at most 12, 13, 14, 15, 16, 17, 18, 19, 20, 21, 22, 23, 24, 25, 26, 27, 28, 29, 30, 31 or 32 amino acids in length.

### Intracellular Domains

In some embodiments, the intracellular signaling domain comprises an intracellular signaling domain derived from a phagocytic receptor. In some embodiments, the intracellular signaling domain comprises an intracellular signaling domain derived from a phagocytic receptor other than a phagocytic receptor selected from Megf10, MerTk, FcRα, or Bai1. In some embodiments, the intracellular signaling domain comprises an intracellular signaling domain derived from a phagocytic receptor selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc-alpha receptor 1, CR1, CD35, CD3ζ, CR3, CR4, Tim-1, Tim-4 and CD169. In some embodiments, the intracellular signaling domain comprises a PI3K recruitment domain. In some embodiments, the intracellular signaling domain comprises an intracellular signaling domain derived from a scavenger receptor. In some embodiments, the intracellular domain comprises a CD47 inhibition domain. In some embodiments, the intracellular domain comprises a Rac inhibition domain, a Cdc42 inhibition domain or a GTPase inhibition domain. In some embodiments, the Rac inhibition domain, the Cdc42 inhibition domain or the GTPase inhibition domain inhibits Rac, Cdc42 or GTPase at a phagocytic cup of a cell expressing the PFP. In some embodiments, the intracellular domain comprises an F-actin disassembly activation domain, a ARHGAP12 activation domain, a ARHGAP25 activation domain or a SH3BP1 activation domain. In some embodiments, the intracellular domain comprises a phosphatase inhibition domain. In some embodiments, the intracellular domain comprises an ARP2/3 inhibition domain. In some embodiments, the intracellular domain comprises at least one ITAM domain. In some embodiments, the intracellular domain comprises at least 1, 2, 3, 4, 5, 6, 7, 8, 9, 10, or more ITAM domains. In some embodiments, the intracellular domain comprises at least one ITAM domain select from an ITAM domain of CD3 zeta, CD3 epsilon, CD3 gamma, CD3 delta, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the at least one ITAM domain comprises a Src-family kinase phosphorylation site. In some embodiments, the at least one ITAM domain comprises a Syk recruitment domain. In some embodiments, the intracellular domain comprises an F-actin depolymerization activation domain. In some embodiments, the intracellular domain lacks enzymatic activity.

In some embodiments, the intracellular domain does not comprise a domain derived from a CD3 zeta intracellular domain. In some embodiments, the intracellular domain does not comprise a domain derived from a MerTK intracellular domain. In some embodiments, the intracellular domain does not comprise a domain derived from a TLR4 intracellular domain. In some embodiments, the intracellular domain comprises a CD47 inhibition domain. In some embodiments, the intracellular signaling domain comprises a domain that activates integrin, such as the intracellular region of PSGL-1.

In some embodiments, the intracellular signaling domain comprises a domain that activates Rap1 GTPase, such as that from EPAC and C3G. In some embodiments, the intracellular signaling domain is derived from paxillin. In some embodiments, the intracellular signaling domain activates focal adhesion kinase. In some embodiments, the intracellular signaling domain is derived from a single phagocytic receptor. In some embodiments, the intracellular signaling domain is derived from a single scavenger receptor. In some embodiments, the intracellular domain comprises a phagocytosis enhancing domain.

In some embodiments, the intracellular domain comprises a pro-inflammatory signaling domain. In some embodiments, the pro-inflammatory signaling domain comprises a kinase activation domain or a kinase binding domain. In some embodiments, the pro-inflammatory signaling domain comprises an IL-1 signaling cascade activation domain. In some embodiments, the pro-inflammatory signaling domain comprises an intracellular signaling domain derived from TLR3, TLR4, TLR7, TLR 9, TRIF, RIG-1, MYD88, MAL, IRAK1, MDA-5, an IFN-receptor, STING, an NLRP family member, NLRP1-14, NOD1, NOD2, Pyrin, AIM2, NLRC4, FCGR3A, FCERIG, CD40, Tank 1-binding kinase (TBK), a caspase domain, a procaspase binding domain or any combination thereof.

In some embodiments, the intracellular domain comprises a signaling domain, such as an intracellular signaling domain, derived from a TLR protein. In some embodiments, the intracellular domain may comprise an intracellular signaling domain of the endo-lysosomal TLR, e.g., TLR3, TLR7, TLR8, or TLR9. In some embodiments, the intracellular signaling domain may be derived from a TLR3 protein. In some embodiments, the intracellular signaling domain may be derived from a TLR7, 8, or 9 protein. In some embodiments, the intracellular domain may comprise an intracellular signaling domain of the cell surface TLRs 1, 2, 4, 5, 6, and 10.

In some embodiments, an intracellular signaling domain is specifically paired with another intracellular domain or a transmembrane domain for maximizing the efficiency and phagocytic potential of the myeloid cell expressing the construct. For example, in some embodiments, a TM domain comprising CD64 TM or a part thereof may be specifically paired with an intracellular signaling domain comprising an innate immune adaptor protein ICD or a PI3kinase recruitment domain, or both. In some embodiments, the combination of domains of the chimeric receptor intracellular domain(s) and/or the transmembrane domains are directed towards maximizing the phagocytosis index of the cell expression the construct, e.g., a myeloid cell. In some embodiments, the combination of domains of the chimeric receptor intracellular domain(s) and/or the transmembrane domains are directed towards maximizing the inflammatory potential of the cell expression the construct such that the cell is capable of lysing the target cell, and activating an immune response pathway for rendering long term immune responsiveness. In some embodiments, the combination of domains of the chimeric receptor intracellular domain(s) and/or the transmembrane domains are directed towards minimizing or obliterating any tonic signaling by the cell expressing the chimeric protein. In some embodiments, the combination of domains of the chimeric receptor intracellular domain(s) and/or the transmembrane domains are directed towards maximizing specificity of the immune response.

In some embodiments, the CFP does not comprise a full length intracellular signaling domain. In some embodiments, the intracellular domain is at least 5, 10, 20, 30, 40, 50, 60, 70, 80, 90, 100, 150, 200, 300, 300, 400, or 500 amino acids in length. In some embodiments, the intracellular domain is at most 10, 20, 30, 40, 50, 60, 70, 80, 90, 100, 150, 200, 300, 300, 400, or 500 amino acids in length.

In some embodiments, the recombinant nucleic acid encodes an FcRα chain extracellular domain, an FcRα chain transmembrane domain and/or an FcRα chain intracellular domain. In some embodiments, the recombinant nucleic acid encodes an FcRβ chain extracellular domain, an FcRβ chain transmembrane domain and/or an FeRβ chain intracellular domain. In some embodiments, the FcRα chain or the FcRβ chain forms a complex with FcRγ when expressed in a cell. In some embodiments, the FcRα chain or FcRβ chain forms a complex with endogenous FcRγ when expressed in a cell. In some embodiments, the FcRα chain or the FcRβ chain does not incorporate into a cell membrane of a cell that does not express FcRγ. In some embodiments, the CFP does not comprise an FcRα chain intracellular signaling domain. In some embodiments, the CFP does not comprise an FcRβ chain intracellular signaling domain. In some embodiments, the recombinant nucleic acid encodes a TREM extracellular domain, a TREM transmembrane domain and/or a TREM intracellular domain. In some embodiments, the TREM is TREM1, TREM 2 or TREM 3.

In some embodiments, the recombinant nucleic acid comprises a sequence encoding a pro-inflammatory polypeptide. In some embodiments, the composition further comprises a proinflammatory nucleotide or a nucleotide in the recombinant nucleic acid, for example, an ATP, ADP, UTP, UDP, and/or UDP-glucose.

### Intracellular Interferon Responsive Domains

Most TLRs activate an adaptor protein called MyD88 activates the transcription-factor protein NF-κB, which drives expression of pro-inflammatory genes as part of the immune response. A subgroup of TLRs (TLR3 and TLR4) can engage the protein TRIF, which acts as a scaffold enabling a kinase enzyme to add a phosphate group to the transcription factor IRF3. This phosphorylation activates IRF3, a member of a family of transcription factors termed interferon regulatory factors (IRFs), which activate broad gene-expression programs. A hallmark of these programs is the production of type I interferon molecules Interferons are potent drivers of a branch of the immune system termed the adaptive immune response, and their presence therefore runs the risk of contributing to autoimmunity. To prevent such an attack by the host's own immune system, an interferon response must be tightly regulated. As a safeguard, a particular sequence of amino-acid residues in TRIF, the pLxIS motif, must be phosphorylated before IRF3 can be activated. This control mechanism provides a ‘licensing step’ that is not specific just for TRIF as an adaptor protein for TLR signaling, but is a general hallmark of sensing pathways that engage IRF3, or the related protein IRF7, to drive interferon expression. Every identified innate sensing pathway connecting the recognition of nucleic acids to the production of type I interferons, with one exception, had been shown previously to signal through one of the three adaptor proteins known so far to contain a pLxIS motif: TRIF, MAVS and STING. Thus, pLxIS-motif-containing adaptor proteins specifically hardwire nucleic-acid recognition to antiviral defenses. In some embodiments, the intracellular signaling domain of the CFP comprises an ICD of an innate immune response protein. In some embodiments, the innate immune response protein is selected from a an intracellular signaling domain derived from TLR3, TLR4, TLR7, TLR 9, TRIF, RIG-1, MYD88, MAL, IRAK1, MDA-5, an IFN-receptor, STING, MAVS, TRIF, TASL, NLRP1, NLRP2, NLRP3, NLRP4, NLRP5, NLRP6, NLRP7, NLRP89, NLRP9, NLRP10, NLRP11, NLRP12, NLRP13, NLRP1-14, NOD1, NOD2, Pyrin, AIM2, NLRC4, FCGR3A, FCERIG, CD40, Tank1-binding kinase (TBK), TNFR1, a chemokine, MHC Class II transactivator (CIITA), IPAF, BIRC1, a RIG-I-like receptor (RLR) protein, macrophage galactose-type lectin (MGL), DC-SIGN (CLEC4L), Langerin (CLEC4K), Myeloid DAP12 associating lectin (MDL)-1 (CLEC5A), a DC associated C type lectin 1 (Dectin 1) subfamily protein, dectin 1/CLEC7A, DNGR1/CLEC9A, Myeloid C type lectin like receptor (MICL) (CLEC12A), CLEC2 (CLEC1B), CLEC12B, a DC immunoreceptor (DCIR) subfamily protein, DCIR/CLEC4A, Dectin 2/CLEC6A, Blood DC antigen 2 (BDCA2) (CLEC4C), and Mincle (macrophage inducible C type lectin) (CLEC4E). In some embodiments, the CFP comprises at least one intracellular signaling domain that comprises an amino acid sequence motif, pLxIS.

In some embodiments, the composition further comprises a pro-inflammatory polypeptide. In some embodiments, the pro-inflammatory polypeptide is a chemokine, cytokine. In some embodiments, the chemokine is selected from the group consisting of IL-1, IL3, IL5, IL-6, il8, IL-12, IL-13, IL-23, TNF, CCL2, CXCL9, CXCL10, CXCL11, IL-18, IL-23, IL-27, CSF, MCSF, GMCSF, IL17, IP-10, RANTES, and interferon. In some embodiments, the cytokine is selected from the group consisting of IL-1, IL3, IL5, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, and interferon.

In some embodiments, the intracellular signaling domains from intracellular adaptor proteins known to be highly active in innate immune defense are incorporated in the chimeric receptor protein. In some embodiments, on or more mutations are introduced in one or more intracellular domains to reduce responsiveness of the intracellular domain to intracellular stimulus that is characteristic of the native intracellular adaptor protein domain, without compromising the effectiveness of the chimeric protein. In some embodiments, such effectiveness is referred to as the enhanced phagocytic potential compared to an identical cell that does not express a chimeric protein. In some embodiments, such effectiveness is referred to as the enhanced inflammatory potential compared to an identical cell that does not express a chimeric protein. In some embodiments, such effectiveness is referred to as the enhanced NF-kappa B activation, or interferon activation in the cell expressing the chimeric protein, compared to an identical cell that does not express a chimeric protein.

### Delivery Vehicles—Nanoparticles

In some embodiments, the myeloid cells are specifically targeted for delivery. Myeloid cells can be targeted using specialized biodegradable polymers, such as PLGA (poly(lactic-co-glycolic) acid and/or polyvinyl alcohol (PVA). In some embodiments, one or more compounds can be selectively incorporated in such polymeric structures to affect the myeloid cell function. In some embodiments, the targeting structures are multilayered, e.g., of one or more PLGA and one or more PVA layers. In some embodiments, the targeting structures are assembled in an order for a layered activity. In some embodiments, the targeted polymeric structures are organized in specific shaped components, such as labile structures that can adhere to a myeloid cell surface and deliver one or more components such as growth factors and cytokines, such as to maintain the myeloid cell in a microenvironment that endows a specific polarization. In some embodiments, the polymeric structures are such that they are not phagocytosed by the myeloid cell, but they can remain adhered on the surface. In some embodiments the one or more growth factors may be M1 polarization factors, such as a cytokine. In some embodiments the one or more growth factors may be an M2 polarization factor, such as a cytokine. In some embodiments, the one or more growth factors may be a macrophage activating cytokine, such as IFNγ. In some embodiments the polymeric structures are capable of sustained release of the one or more growth factors in an in vivo environment, such as in a solid tumor.

In some embodiments, the recombinant nucleic acid comprises a sequence encoding a homeostatic regulator of inflammation. In some embodiments, the homeostatic regulator of inflammation is a sequence in an untranslated region (UTR) of an mRNA. In some embodiments, the sequence in the UTR is a sequence that binds to an RNA binding protein. In some embodiments, translation is inhibited or prevented upon binding of the RNA binding protein to the sequence in an untranslated region (UTR). In some embodiments the RNA has a poly A tail. In some embodiments, the RNA (mRNA) has a poly A tail that has 200-1000 A (adenosine) residues. In some embodiments the mRNA has a poly A tail of about 200-800 A residues. In some embodiments the mRNA has a poly A tail of about 800-1200 A residues. In some embodiments the mRNA has a poly A tail of about, or at the most 1500 A residues.

In some embodiments, the target cell is a mammalian cell. In some embodiments, the target cell is a human cell. In some embodiments, the target cell comprises a cell infected with a pathogen. In some embodiments, the target cell is a cancer cell. In some embodiments, the target cell is a cancer cell that is a lymphocyte. In some embodiments, the target cell is a cancer cell that is an ovarian cancer cell. In some embodiments, the target cell is a cancer cell that is a breast cell. In some embodiments, the target cell is a cancer cell that is a pancreatic cell. In some embodiments, the target cell is a cancer cell that is a glioblastoma cell.

In some embodiments, the recombinant nucleic acid is DNA. In some embodiments, the recombinant nucleic acid is RNA. In some embodiments, the recombinant nucleic acid is mRNA. In some embodiments, the recombinant nucleic acid is an unmodified mRNA. In some embodiments, the recombinant nucleic acid is a modified mRNA. In some embodiments, the recombinant nucleic acid is a circRNA. In some embodiments, the recombinant nucleic acid is a tRNA. In some embodiments, the recombinant nucleic acid is a microRNA.

Also provided herein is a vector comprising a recombinant nucleic acid sequence encoding a CFP described herein. In some embodiments, the vector is viral vector. In some embodiments, the viral vector is retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′UTR and/or a 3′UTR flanking the at least one nucleic acid sequence encoding one or more polypeptides. In some embodiments, the vector further comprises one or more regulatory regions.

Also provided herein is a polypeptide encoded by the recombinant nucleic acid of a composition described herein.

Provided herein is a composition comprising a recombinant nucleic acid sequence encoding a CFP comprising a phagocytic or tethering receptor (PR) subunit (e.g., a phagocytic receptor fusion protein (PFP)) comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein upon binding of the CFP to the antigen of the target cell, the killing or phagocytosis activity of a myeloid cell, such as a neutrophil, monocyte, myeloid dendritic cell (mDC), mast cell or macrophage expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP.

TABLE 1B
Exemplary Chimeric Fusion Protein
Signal Peptide Sequences

| SEQ | | |
|---|---|---|
| ID NO | CFP/Domain | Sequence |
| 7 | GMCSF Signal | MWLQSLLLLGTVACSIS |
| | peptide | |

TABLE 1C
Exemplary Chimeric Fusion Protein Extracellular
(ECD)/Hinge Domains

| SEQ | | |
|---|---|---|
| ID NO | CFP/Domain | Sequence |
| 8 | CD89 ECD | DSIHQDYTTQN |
| 9 | Linker | SGGGGAAADYKDDDDKGS |
| 10 | Linker-CD89 | SGGGGAAADYKDDDDKGSDSIHQDYTTQN |
| | ECD | |

TABLE ID
Exemplary Chimeric Fusion Protein
Transmembrane Domains (TMDs)

| SEQ | | |
|---|---|---|
| ID NO | CFP/Domain | Sequence |
| 11 | CD89 TMD | LIRMAVAGLVLVALLAILV |

TABLE 2
Exemplary Chimeric Fusion Protein Intracellular Signaling Domains (ICDs)

| SEQ | | |
|---|---|---|
| ID | | |
| NO | CFP/Domain | Sequence |
| 12 | CD89 | ENWHSHTALNKEASADVAEPSWSQQMCQPGLTFARTPSVCK |
| | intracellular | |
| | domain | |
| 13 | Fc-epsilon | LYCRRLKIQVRKAAITSYEKSDGVYTGLSTRNQETYETLKHEK |
| | receptor subunit | PPQ |
| | gamma | |
| | (FCER1G) | |
| | intracellular | |
| | signaling | |
| | domain | |
| 14 | FCER1G | LYCRLKIQVRKAAITSYEKSDGVYTGLSTRNQETYETLKHEKP |
| | intracellular | PQ |
| | signaling | |
| | domain | |
| 15 | FCER1G | RLKIQVRKAAITSYEKSDGVYTGLSTRNQETYETLKHEKPPQ |
| | intracellular | |
| | signaling | |
| | domain | |
| 16 | FCER1G | RRLKIQVRKAAITSYEKSDGVYTGLSTRNQETYETLKHEKPPQ |
| | intracellular | |
| | signaling | |
| | domain | |
| 17 | PI3K | YEDMRGILYAAPQLRSIRGQPGPNHEEDADSYENM |
| | recruitment | |
| | domain | |
| 18 | CD40 | KKVAKKPTNKAPHPKQEPQEINFPDDLPGSNTAAPVQETLHGC |
| | intracellular | QPVTQEDGKESRISVQERQ |
| | signaling | |
| | domain | |
| 19 | TRIF (1-255) | MACTGPSLPSAFDILGAAGQDKLLYLKHKLKTPRPGCQGQDL |
| | intracellular | LHAMVLLKLGQETEARISLEALKADAVARLVARQWAGVDST |
| | signaling | EDPEEPPDVSWAVARLYHLLAEEKLCPASLRDVAYQEAVRTL |
| | domain | SSRDDHRLGELQDEARNRCGWDIAGDPGSIRTLQSNLGCLPPS |
| | | SALPSGTRSLPRPIDGVSDWSQGCSLRSTGSPASLASNLEISQSP |
| | | TMPFLSLHRSPHGPSKLCDDPQASLVPEPVPGGCQEPEEMSW |
| 20 | TRIF (short) | GSIRTLQSNLGCLPPSSALPSGTRSLPRPIDGVSDWSQGCSLRST |
| | (153-387) | GSPASLASNLEISQSPTMPFLSLHRSPHGPSKLCDDPQASLVPEP |
| | intracellular | VPGGCQEPEEMSWPPSGEIASPPELPSSPPPGLPEVAPDATSTGL |
| | signaling | PDTPAAPETSTNYPVECTEGSAGPQSLPLPILEPVKNPCSVKDQ |
| | domain | TPLQLSVEDTTSPNTKPCPPTPTTPETSPPPPPPPPSSTPCSAHLT |
| | | PSSLFPSSLE |
| 21 | TRIF (short) | RGSIRTLQSNLGCLPPSSALPSGTRSLPRPIDGVSDWSQGCSLRS |
| | (R-153-387) | TGSPASLASNLEISQSPTMPFLSLHRSPHGPSKLCDDPQASLVPE |
| | intracellular | PVPGGCQEPEEMSWPPSGEIASPPELPSSPPPGLPEVAPDATSTG |
| | signaling | LPDTPAAPETSTNYPVECTEGSAGPQSLPLPILEPVKNPCSVKD |
| | domain | QTPLQLSVEDTTSPNTKPCPPTPTTPETSPPPPPPPPSSTPCSAHL |
| | | TPSSLFPSSLE |
| 22 | TRIF (long) | GSIRTLQSNLGCLPPSSALPSGTRSLPRPIDGVSDWSQGCSLRST |
| | (153-545) | GSPASLASNLEISQSPTMPFLSLHRSPHGPSKLCDDPQASLVPEP |
| | intracellular | VPGGCQEPEEMSWPPSGEIASPPELPSSPPPGLPEVAPDATSTGL |
| | signaling | PDTPAAPETSTNYPVECTEGSAGPQSLPLPILEPVKNPCSVKDQ |
| | domain | TPLQLSVEDTTSPNTKPCPPTPTTPETSPPPPPPPPSSTPCSAHLT |
| | | PSSLFPSSLESSSEQKFYNFVILHARADEHIALRVREKLEALGVP |
| | | DGATFCEDFQVPGRGELSCLQDAIDHSAFIILLLTSNFDCRLSL |
| | | HQVNQAMMSNLTRQGSPDCVIPFLPLESSPAQLSSDTASLLSG |
| | | LVRLDEHSQIFARKVANTFKPHRLQARKAMWRKEQD |

TABLE 3
Exemplary Chimeric Fusion Protein Intracellular Domains

| SEQ | | |
|---|---|---|
| ID | | |
| NO | CFP/Domain | Sequence |
| 23 | CD40-GS | KKVAKKPTNKAPHPKQEPQEINFPDDLPGSNTAAPVQETLHGC |
| | linker- | QPVTQEDGKESRISVQERQGSRLKIQVRKAAITSYEKSDGVYT |
| | FCERIG | GLSTRNQETYETLKHEKPPQ |
| 24 | FCERIG-GS | RLKIQVRKAAITSYEKSDGVYTGLSTRNQETYETLKHEKPPQG |
| | linker-TRIF | SGSIRTLQSNLGCLPPSSALPSGTRSLPRPIDGVSDWSQGCSLRS |
| | (short) | TGSPASLASNLEISQSPTMPFLSLHRSPHGPSKLCDDPQASLVPE |
| | (153-387) | PVPGGCQEPEEMSWPPSGEIASPPELPSSPPPGLPEVAPDATSTG |
| | | LPDTPAAPETSTNYPVECTEGSAGPQSLPLPILEPVKNPCSVKD |
| | | QTPLQLSVEDTTSPNTKPCPPTPTTPETSPPPPPPPPSSTPCSAHL |
| | | TPSSLFPSSLE |
| 25 | FCERIG-GS | RLKIQVRKAAITSYEKSDGVYTGLSTRNQETYETLKHEKPPQG |
| | linker-PI3K | SYEDMRGILYAAPQLRSIRGQPGPNHEEDADSYENM |
| | recruitment | |
| | domain | |

TABLE 4
Exemplary Chimeric Fusion Protein Sequences

| SEQ | | |
|---|---|---|
| ID | | |
| NO | CFP/Domain | Sequence |
| 26 | TROP2-CD89 | QVQLQQSGSELKKPGASVKVSCKASGYTFTNYGMNWVKQAP |
| | | GQGLKWMGWINTYTGEPTYTDDFKGRFAFSLDTSVSTAYLQI |
| | | SSLKADDTAVYFCARGGFGSSYWYFDVWGQGSLVTVSSGGG |
| | | GSGGGGSGGGGSDIQLTQSPSSLSASVGDRVSITCKASQDVSIA |
| | | VAWYQQKPGKAPKLLIYSASYRYTGVPDRFSGSGSGTDFTLTI |
| | | SSLQPEDFAVYYCQQHYITPLTFGAGTKVEIKRGSGGSDSIHQD |
| | | YTTQNLIRMAVAGLVLVALLAILVENWHSHTALNKEASADVA |
| | | EPSWSQQMCQPGLTFARTPSVCK |
| 27 | TROP2-CD89 | QVQLQQSGSELKKPGASVKVSCKASGYTFTNYGMNWVKQAP |
| | | GQGLKWMGWINTYTGEPTYTDDFKGRFAFSLDTSVSTAYLQI |
| | | SSLKADDTAVYFCARGGFGSSYWYFDVWGQGSLVTVSSGGG |
| | | GSGGGGSGGGGSDIQLTQSPSSLSASVGDRVSITCKASQDVSIA |
| | | VAWYQQKPGKAPKLLIYSASYRYTGVPDRFSGSGSGTDFTLTI |
| | | SSLQPEDFAVYYCQQHYITPLTFGAGTKVEIKRSGGGGAAADY |
| | | KDDDDKGSDSIHQDYTTQNLIRMAVAGLVLVALLAILVENWH |
| | | SHTALNKEASADVAEPSWSQQMCQPGLTFARTPSVCK |
| 28 | TROP2-CD89- | QVQLQQSGSELKKPGASVKVSCKASGYTFTNYGMNWVKQAP |
| | FcR | GQGLKWMGWINTYTGEPTYTDDFKGRFAFSLDTSVSTAYLQI |
| | | SSLKADDTAVYFCARGGFGSSYWYFDVWGQGSLVTVSSGGG |
| | | GSGGGGSGGGGSDIQLTQSPSSLSASVGDRVSITCKASQDVSIA |
| | | VAWYQQKPGKAPKLLIYSASYRYTGVPDRFSGSGSGTDFTLTI |
| | | SSLQPEDFAVYYCQQHYITPLTFGAGTKVEIKRSGGGGAAADY |
| | | KDDDDKGSDSIHQDYTTQNLIRMAVAGLVLVALLAILVRLKIQ |
| | | VRKAAITSYEKSDGVYTGLSTRNQETYETLKHEKPPQ |
| 29 | TROP2-CD89- | QVQLQQSGSELKKPGASVKVSCKASGYTFTNYGMNWVKQAP |
| | FcR-PI3K | GQGLKWMGWINTYTGEPTYTDDFKGRFAFSLDTSVSTAYLQI |
| | | SSLKADDTAVYFCARGGFGSSYWYFDVWGQGSLVTVSSGGG |
| | | GSGGGGSGGGGSDIQLTQSPSSLSASVGDRVSITCKASQDVSIA |
| | | VAWYQQKPGKAPKLLIYSASYRYTGVPDRFSGSGSGTDFTLTI |
| | | SSLQPEDFAVYYCQQHYITPLTFGAGTKVEIKRSGGGGAAADY |
| | | KDDDDKGSDSIHQDYTTQNLIRMAVAGLVLVALLAILVRLKIQ |
| | | VRKAAITSYEKSDGVYTGLSTRNQETYETLKHEKPPQGSYED |
| | | MRGILYAAPQLRSIRGQPGPNHEEDADSYENM |
| 30 | TROP2-CD89- | QVQLQQSGSELKKPGASVKVSCKASGYTFTNYGMNWVKQAP |
| | CD40 | GQGLKWMGWINTYTGEPTYTDDFKGRFAFSLDTSVSTAYLQI |
| | | SSLKADDTAVYFCARGGFGSSYWYFDVWGQGSLVTVSSGGG |
| | | GSGGGGSGGGGSDIQLTQSPSSLSASVGDRVSITCKASQDVSIA |
| | | VAWYQQKPGKAPKLLIYSASYRYTGVPDRFSGSGSGTDFTLTI |
| | | SSLQPEDFAVYYCQQHYITPLTFGAGTKVEIKRSGGGGAAADY |
| | | KDDDDKGSDSIHQDYTTQNLIRMAVAGLVLVALLAILVKKVA |
| | | KKPTNKAPHPKQEPQEINFPDDLPGSNTAAPVQETLHGCQPVT |
| | | QEDGKESRISVQERQ |
| 31 | TROP2-CD89- | QVQLQQSGSELKKPGASVKVSCKASGYTFTNYGMNWVKQAP |
| | CD40-FcR | GQGLKWMGWINTYTGEPTYTDDFKGRFAFSLDTSVSTAYLQI |
| | | SSLKADDTAVYFCARGGFGSSYWYFDVWGQGSLVTVSSGGG |
| | | GSGGGGSGGGGSDIQLTQSPSSLSASVGDRVSITCKASQDVSIA |
| | | VAWYQQKPGKAPKLLIYSASYRYTGVPDRFSGSGSGTDFTLTI |
| | | SSLQPEDFAVYYCQQHYITPLTEGAGTKVEIKRSGGGGAAADY |
| | | KDDDDKGSDSIHQDYTTQNLIRMAVAGLVLVALLAILVKKVA |
| | | KKPTNKAPHPKQEPQEINFPDDLPGSNTAAPVQETLHGCQPVT |
| | | QEDGKESRISVQERQGSRLKIQVRKAAITSYEKSDGVYTGLST |
| | | RNQETYETLKHEKPPQ |
| 32 | TROP2-CD89- | QVQLQQSGSELKKPGASVKVSCKASGYTFTNYGMNWVKQAP |
| | TRIF | GQGLKWMGWINTYTGEPTYTDDFKGRFAFSLDTSVSTAYLQI |
| | | SSLKADDTAVYFCARGGFGSSYWYFDVWGQGSLVTVSSGGG |
| | | GSGGGGSGGGGSDIQLTQSPSSLSASVGDRVSITCKASQDVSIA |
| | | VAWYQQKPGKAPKLLIYSASYRYTGVPDRFSGSGSGTDFTLTI |
| | | SSLQPEDFAVYYCQQHYITPLTFGAGTKVEIKRSGGGGAAADY |
| | | KDDDDKGSDSIHQDYTTQNLIRMAVAGLVLVALLAILVRGSIR |
| | | TLQSNLGCLPPSSALPSGTRSLPRPIDGVSDWSQGCSLRSTGSP |
| | | ASLASNLEISQSPTMPFLSLHRSPHGPSKLCDDPQASLVPEPVP |
| | | GGCQEPEEMSWPPSGEIASPPELPSSPPPGLPEVAPDATSTGLPD |
| | | TPAAPETSTNYPVECTEGSAGPQSLPLPILEPVKNPCSVKDQTP |
| | | LQLSVEDTTSPNTKPCPPTPTTPETSPPPPPPPPSSTPCSAHLTP |
| | | SSLFPSSLE |
| 33 | TROP2-CD89- | QVQLQQSGSELKKPGASVKVSCKASGYTFTNYGMNWVKQAP |
| | FcR-TRIF | GQGLKWMGWINTYTGEPTYTDDFKGRFAFSLDTSVSTAYLQI |
| | | SSLKADDTAVYFCARGGFGSSYWYFDVWGQGSLVTVSSGGG |
| | | GSGGGGSGGGGSDIQLTQSPSSLSASVGDRVSITCKASQDVSIA |
| | | VAWYQQKPGKAPKLLIYSASYRYTGVPDRFSGSGSGTDFTLTI |
| | | SSLQPEDFAVYYCQQHYITPLTFGAGTKVEIKRSGGGGAAADY |
| | | KDDDDKGSDSIHQDYTTQNLIRMAVAGLVLVALLAILVRLKIQ |
| | | VRKAAITSYEKSDGVYTGLSTRNQETYETLKHEKPPQGSGSIR |
| | | TLQSNLGCLPPSSALPSGTRSLPRPIDGVSDWSQGCSLRSTGSP |
| | | ASLASNLEISQSPTMPFLSLHRSPHGPSKLCDDPQASLVPEPVP |
| | | GGCQEPEEMSWPPSGEIASPPELPSSPPPGLPEVAPDATSTGLPD |
| | | TPAAPETSTNYPVECTEGSAGPQSLPLPILEPVKNPCSVKDQTP |
| | | LQLSVEDTTSPNTKPCPPTPTTPETSPPPPPPPPSSTPCSAHLTP |
| | | SSLFPSSLE |

TABLE 5
Exemplary TROP2 binder sequences (CDR sequences are underlined)

| Structure | Domain | Sequence |
|---|---|---|
| Anti- | MWLQSLLLLGTVACSISQVQLQQSGSELKKPGASVKVSCKASGYTFTNYGMN | |
| TROP2- | WVKQAPGQGLKWMGWINTYTGEPTYTDDFKGRFAFSLDTSVSTAYLQISSLK | |
| | ADDTAVYFCARGGFGSSYWYFDVWGQGSLVTVSSGGGGSGGGGSGGGGSDI | |
| | QLTQSPSSLSASVGDRVSITCKASQDVSIAVAWYQQKPGKAPKLLIYSASYRY | |
| | TGVPDRFSGSGSGTDFTLTISSLOPEDFAVYYCQQHYITPLTFGAGTKVEIKRS | |
| | GGGGAAADYKDDDDKGSDSIHQDYTTQNLIRMAVAGLVLVALLAILVENWH | |
| | SHTALNKEASADVAEPSWSQQMCQPGLTFARTPSVCK (SEQ ID NO: 34) | |
| CD89 | Signal peptide | MWLQSLLLLGTVACSIS |
| | Anti-TROP2 | QVQLQQSGSELKKPGASVKVSCKASGYTFTNYGMNWVKQAP |
| | domain | GQGLKWMGWINTYTGEPTYTDDFKGRFAFSLDTSVSTAYLQIS |
| | | SLKADDTAVYFCARGGFGSSYWYFDVWGQGSLVTVSSGGGG |
| | | SGGGGSGGGGSDIQLTQSPSSLSASVGDRVSITCKASQDVSIAV |
| | | AWYQQKPGKAPKLLIYSASYRYTGVPDRFSGSGSGTDFTLTISS |
| | | LQPEDFAVYYCQQHYITPLTFGAGTKVEIKR |
| | linker | SGGGGAAADYKDDDDKGS |
| | CD89 TMD | LIRMAVAGLVLVALLAILV |
| | CD89 TMD | DSIHQDYTTQNLIRMAVAGLVLVALLAILVENWHSHTALNKE |
| | and | ASADVAEPSWSQQMCQPGLTFARTPSVCK |
| | intracellular | |
| | domain | |
| Anti- | MWLQSLLLLGTVACSISQVQLQQSGSELKKPGASVKVSCKASGYTFTNYGMN | |
| TROP2- | WVKQAPGQGLKWMGWINTYTGEPTYTDDFKGRFAFSLDTSVSTAYLQISSLK | |
| CD89- | ADDTAVYFCARGGFGSSYWYFDVWGQGSLVTVSSGGGGSGGGGSGGGGSDI | |
| FcR | QLTQSPSSLSASVGDRVSITCKASQDVSIAVAWYQQKPGKAPKLLIYSASYRY | |
| | TGVPDRFSGSGSGTDFTLTISSLQPEDFAVYYCQQHYITPLTFGAGTKVEIKRS | |
| | GGGGAAADYKDDDDKGSDSIHQDYTTQNLIRMAVAGLVLVALLAILVRLKIQ | |
| | VRKAAITSYEKSDGVYTGLSTRNQETYETLKHEKPPQ (SEQ ID NO: 35) | |
| | Signal peptide | MWLQSLLLLGTVACSIS |
| | Anti-TROP2 | QVQLQQSGSELKKPGASVKVSCKASGYTFTNYGMNWVKQAP |
| | domain | GQGLKWMGWINTYTGEPTYTDDFKGRFAFSLDTSVSTAYLQIS |
| | | SLKADDTAVYFCARGGFGSSYWYFDVWGQGSLVTVSSGGGG |
| | | SGGGGSGGGGSDIQLTQSPSSLSASVGDRVSITCKASQDVSIAV |
| | | AWYQQKPGKAPKLLIYSASYRYTGVPDRFSGSGSGTDFTLTISS |
| | | LQPEDFAVYYCQQHYITPLTFGAGTKVEIKR |
| | linker | SGGGGAAADYKDDDDKGS |
| | CD89 TMD | DSIHQDYTTQNLIRMAVAGLVLVALLAILV |
| | FcR | RLKIQVRKAAITSYEKSDGVYTGLSTRNQETYETLKHEKPPQ |
| | intracellular | |
| | domain (ICD) | |
| Anti- | MWLQSLLLLGTVACSISQVQLQQSGSELKKPGASVKVSCKASGYTFTNYGMN | |
| TROP2- | WVKQAPGQGLKWMGWINTYTGEPTYTDDFKGRFAFSLDTSVSTAYLQISSLK | |
| CD89- | ADDTAVYFCARGGFGSSYWYFDVWGQGSLVTVSSGGGGSGGGGSGGGGSDI | |
| FcR-PI3K | QLTQSPSSLSASVGDRVSITCKASQDVSIAVAWYQQKPGKAPKLLIYSASYRY | |
| | TGVPDRFSGSGSGTDFTLTISSLQPEDFAVYYCQQHYITPLTFGAGTKVEIKRS | |
| | GGGGAAADYKDDDDKGSDSIHQDYTTQNLIRMAVAGLVLVALLAILVRLKIQ | |
| | VRKAAITSYEKSDGVYTGLSTRNQETYETLKHEKPPQGSYEDMRGILYAAPQL | |
| | RSIRGQPGPNHEEDADSYENM (SEQ ID NO: 36) | |
| | Signal peptide | MWLQSLLLLGTVACSIS |
| | Anti-TROP2 | QVQLQQSGSELKKPGASVKVSCKASGYTFTNYGMNWVKQAP |
| | domain | GQGLKWMGWINTYTGEPTYTDDFKGRFAFSLDTSVSTAYLQIS |
| | | SLKADDTAVYFCARGGFGSSYWYFDVWGQGSLVTVSSGGGG |
| | | SGGGGSGGGGSDIQLTQSPSSLSASVGDRVSITCKASQDVSIAV |
| | | AWYQQKPGKAPKLLIYSASYRYTGVPDRFSGSGSGTDFTLTISS |
| | | LQPEDFAVYYCQQHYITPLTFGAGTKVEIKR |
| | Linker | SGGGGAAADYKDDDDKGS |
| | CD89 TMD | DSIHQDYTTQNLIRMAVAGLVLVALLAILV |
| | FcR | RLKIQVRKAAITSYEKSDGVYTGLSTRNQETYETLKHEKPPQ |
| | intracellular | |
| | domain (ICD) | |
| | Linker | GS |
| | PI3K | YEDMRGILYAAPQLRSIRGQPGPNHEEDADSYENM |
| | recruitment | |
| | domain | |
| | (PI3K) ICD | |
| Anti- | MWLQSLLLLGTVACSISQVQLQQSGSELKKPGASVKVSCKASGYTFTNYGMN | |
| TROP2- | WVKQAPGQGLKWMGWINTYTGEPTYTDDFKGRFAFSLDTSVSTAYLQISSLK | |
| CD89- | ADDTAVYFCARGGFGSSYWYFDVWGQGSLVTVSSGGGGSGGGGSGGGGSDI | |
| CD40 | QLTQSPSSLSASVGDRVSITCKASQDVSIAVAWYQQKPGKAPKLLIYSASYRY | |
| | TGVPDRFSGSGSGTDFTLTISSLQPEDFAVYYCQQHYITPLTFGAGTKVEIKRS | |
| | GGGGAAADYKDDDDKGSDSIHQDYTTQNLIRMAVAGLVLVALLAILVKKVA | |
| | KKPTNKAPHPKQEPQEINFPDDLPGSNTAAPVQETLHGCQPVTQEDGKESRIS | |
| | VQERQ (SEQ ID NO: 37) | |
| | Leader | MWLQSLLLLGTVACSIS |
| | Anti-TROP2 | QVQLQQSGSELKKPGASVKVSCKASGYTFTNYGMNWVKQAP |
| | domain | GQGLKWMGWINTYTGEPTYTDDFKGRFAFSLDTSVSTAYLQIS |
| | | SLKADDTAVYFCARGGFGSSYWYFDVWGQGSLVTVSSGGGG |
| | | SGGGGSGGGGSDIQLTQSPSSLSASVGDRVSITCKASQDVSIAV |
| | | AWYQQKPGKAPKLLIYSASYRYTGVPDRFSGSGSGTDFTLTISS |
| | | LQPEDFAVYYCQQHYITPLTFGAGTKVEIKR |
| | Linker | SGGGGAAADYKDDDDKGS |
| | CD89 TMD | DSIHQDYTTQNLIRMAVAGLVLVALLAILV |
| | CD40 ICD | KKVAKKPTNKAPHPKQEPQEINFPDDLPGSNTAAPVQETLHGC |
| | | QPVTQEDGKESRISVQERQ |
| Anti- | MWLQSLLLLGTVACSISQVQLQQSGSELKKPGASVKVSCKASGYTFTNYGMN | |
| TROP2- | WVKQAPGQGLKWMGWINTYTGEPTYTDDFKGRFAFSLDTSVSTAYLQISSLK | |
| CD89- | ADDTAVYFCARGGFGSSYWYFDVWGQGSLVTVSSGGGGSGGGGSGGGGSDI | |
| CD40- | QLTQSPSSLSASVGDRVSITCKASQDVSIAVAWYQQKPGKAPKLLIYSASYRY | |
| FcR | TGVPDRFSGSGSGTDFTLTISSLQPEDFAVYYCQQHYITPLTFGAGTKVEIKRS | |
| | GGGGAAADYKDDDDKGSDSIHQDYTTQNLIRMAVAGLVLVALLAILVKKVA | |
| | KKPTNKAPHPKQEPQEINFPDDLPGSNTAAPVQETLHGCQPVTQEDGKESRIS | |
| | VQERQGSRLKIQVRKAAITSYEKSDGVYTGLSTRNQETYETLKHEKPPQ (SEQ | |
| | ID NO: 38) | |
| | Signal peptide | MWLQSLLLLGTVACSIS |
| | Anti-TROP2 | QVQLQQSGSELKKPGASVKVSCKASGYTFTNYGMNWVKQAP |
| | domain | GQGLKWMGWINTYTGEPTYTDDFKGRFAFSLDTSVSTAYLQIS |
| | | SLKADDTAVYFCARGGFGSSYWYFDVWGQGSLVTVSSGGGG |
| | | SGGGGSGGGGSDIQLTQSPSSLSASVGDRVSITCKASQDVSIAV |
| | | AWYQQKPGKAPKLLIYSASYRYTGVPDRFSGSGSGTDFTLTISS |
| | | LQPEDFAVYYCQQHYITPLTFGAGTKVEIKR |
| | Linker | SGGGGAAADYKDDDDKGS |
| | CD89 TMD | DSIHQDYTTQNLIRMAVAGLVLVALLAILV |
| | CD40 ICD | KKVAKKPTNKAPHPKQEPQEINFPDDLPGSNTAAPVQETLHGC |
| | | QPVTQEDGKESRISVQERQ |
| | Linker | GS |
| | FcR ICD | RLKIQVRKAAITSYEKSDGVYTGLSTRNQETYETLKHEKPPQ |
| Anti- | MWLQSLLLLGTVACSISQVQLQQSGSELKKPGASVKVSCKASGYTFTNYGMN | |
| TROP2- | WVKQAPGQGLKWMGWINTYTGEPTYTDDFKGRFAFSLDTSVSTAYLQISSLK | |
| CD89- | ADDTAVYFCARGGFGSSYWYFDVWGQGSLVTVSSGGGGSGGGGSGGGGSDI | |
| TRIF | QLTQSPSSLSASVGDRVSITCKASQDVSIAVAWYQQKPGKAPKLLIYSASYRY | |
| | TGVPDRFSGSGSGTDFTLTISSLQPEDFAVYYCQQHYITPLTFGAGTKVEIKRS | |
| | GGGGAAADYKDDDDKGSDSIHQDYTTQNLIRMAVAGLVLVALLAILVRGSIR | |
| | TLQSNLGCLPPSSALPSGTRSLPRPIDGVSDWSQGCSLRSTGSPASLASNLEISQ | |
| | SPTMPFLSLHRSPHGPSKLCDDPQASLVPEPVPGGCQEPEEMSWPPSGEIASPP | |
| | ELPSSPPPGLPEVAPDATSTGLPDTPAAPETSTNYPVECTEGSAGPQSLPLPILEP | |
| | VKNPCSVKDQTPLQLSVEDTTSPNTKPCPPTPTTPETSPPPPPPPPSSTPCSAHLT | |
| | PSSLFPSSLE (SEQ ID NO: 39) | |
| | Signal peptide | MWLQSLLLLGTVACSIS |
| | Anti-TROP2 | QVQLQQSGSELKKPGASVKVSCKASGYTFTNYGMNWVKQAP |
| | domain | GQGLKWMGWINTYTGEPTYTDDFKGRFAFSLDTSVSTAYLQIS |
| | | SLKADDTAVYFCARGGFGSSYWYFDVWGQGSLVTVSSGGGG |
| | | SGGGGSGGGGSDIQLTQSPSSLSASVGDRVSITCKASQDVSIAV |
| | | AWYQQKPGKAPKLLIYSASYRYTGVPDRFSGSGSGTDFTLTISS |
| | | LQPEDFAVYYCQQHYITPLTFGAGTKVEIKR |
| | Linker | SGGGGAAADYKDDDDKGS |
| | CD89 TMD | DSIHQDYTTQNLIRMAVAGLVLVALLAILV |
| | TRIF-ICD | RGSIRTLQSNLGCLPPSSALPSGTRSLPRPIDGVSDWSQGCSLRS |
| | | TGSPASLASNLEISQSPTMPFLSLHRSPHGPSKLCDDPQASLVPE |
| | | PVPGGCQEPEEMSWPPSGEIASPPELPSSPPPGLPEVAPDATSTG |
| | | LPDTPAAPETSTNYPVECTEGSAGPQSLPLPILEPVKNPCSVKD |
| | | QTPLQLSVEDTTSPNTKPCPPTPTTPETSPPPPPPPPSSTPCSAHL |
| | | TPSSLFPSSLE |
| Anti- | MWLQSLLLLGTVACSISQVQLQQSGSELKKPGASVKVSCKASGYTFTNYGMN | |
| TROP2- | WVKQAPGQGLKWMGWINTYTGEPTYTDDFKGRFAFSLDTSVSTAYLQISSLK | |
| CD89- | ADDTAVYFCARGGFGSSYWYFDVWGQGSLVTVSSGGGGSGGGGSGGGGSDI | |
| FcR- | QLTQSPSSLSASVGDRVSITCKASQDVSIAVAWYQQKPGKAPKLLIYSASYRY | |
| TRIF | TGVPDRFSGSGSGTDFTLTISSLQPEDFAVYYCQQHYITPLTFGAGTKVEIKRS | |
| | GGGGAAADYKDDDDKGSDSIHQDYTTQNLIRMAVAGLVLVALLAILVRLKIQ | |
| | VRKAAITSYEKSDGVYTGLSTRNQETYETLKHEKPPQGSGSIRTLQSNLGCLPP | |
| | SSALPSGTRSLPRPIDGVSDWSQGCSLRSTGSPASLASNLEISQSPTMPFLSLHR | |
| | SPHGPSKLCDDPQASLVPEPVPGGCQEPEEMSWPPSGEIASPPELPSSPPPGLPE | |
| | VAPDATSTGLPDTPAAPETSTNYPVECTEGSAGPQSLPLPILEPVKNPCSVKDQ | |
| | TPLQLSVEDTTSPNTKPCPPTPTTPETSPPPPPPPPSSTPCSAHLTPSSLFPSSLE | |
| | (SEQ ID NO: 40) | |
| | Signal peptide | MWLQSLLLLGTVACSIS |
| | Anti-TROP2 | QVQLQQSGSELKKPGASVKVSCKASGYTFTNYGMNWVKQAP |
| | domain | GQGLKWMGWINTYTGEPTYTDDFKGRFAFSLDTSVSTAYLQIS |
| | | SLKADDTAVYFCARGGFGSSYWYFDVWGQGSLVTVSSGGGG |
| | | SGGGGSGGGGSDIQLTQSPSSLSASVGDRVSITCKASQDVSIAV |
| | | AWYQQKPGKAPKLLIYSASYRYTGVPDRFSGSGSGTDFTLTISS |
| | | LQPEDFAVYYCQQHYITPLTFGAGTKVEIKR |
| | Linker | SGGGGAAADYKDDDDKGS |
| | CD89 TMD | DSIHQDYTTQNLIRMAVAGLVLVALLAILV |
| | FcR ICD | RLKIQVRKAAITSYEKSDGVYTGLSTRNQETYETLKHEKPPQ |
| | Linker | GS |
| | TRIF-ICD | GSIRTLQSNLGCLPPSSALPSGTRSLPRPIDGVSDWSQGCSLRST |
| | | GSPASLASNLEISQSPTMPFLSLHRSPHGPSKLCDDPQASLVPEP |
| | | VPGGCQEPEEMSWPPSGEIASPPELPSSPPPGLPEVAPDATSTGL |
| | | PDTPAAPETSTNYPVECTEGSAGPQSLPLPILEPVKNPCSVKDQ |
| | | TPLQLSVEDTTSPNTKPCPPTPTTPETSPPPPPPPPSSTPCSAHLTP |
| | | SSLFPSSLE |
| Anti- | MWLQSLLLLGTVACSISQVQLQQSGSELKKPGASVKVSCKASGYTFTNYGMNW | |
| TROP2- | VKQAPGQGLKWMGWINTYTGEPTYTDDFKGRFAFSLDTSVSTAYLQISSLKADD | |
| CD89 | TAVYFCARGGFGSSYWYFDVWGQGSLVTVSSGGGGSGGGGSGGGGSDIQLTQSP | |
| | SSLSASVGDRVSITCKASQDVSIAVAWYQQKPGKAPKLLIYSASYRYTGVPDRFS | |
| | GSGSGTDFTLTISSLQPEDFAVYYCQQHYITPLTEGAGTKVEIKRGSGGSDSIHQDY | |
| | TTQNLIRMAVAGLVLVALLAILVENWHSHTALNKEASADVAEPSWSQQMCQPGLTF | |
| | ARTPSVCK (SEQ ID NO: 41) | |
| | Signal peptide | MWLQSLLLLGTVACSIS |
| | Anti-TROP2 | QVQLQQSGSELKKPGASVKVSCKASGYTFTNYGMNWVKQAPGQ |
| | scFv VH | GLKWMGWINTYTGEPTYTDDFKGRFAFSLDTSVSTAYLQISSLKA |
| | domain | DDTAVYFCARGGFGSSYWYEDVWGQGSLVTVSS |
| | Linker 1 | GGGGSGGGGSGGGGS |
| | Anti-TROP2 | DIQLTQSPSSLSASVGDRVSITCKASQDVSIAVAWYQQKPGKAPK |
| | scFv VL | LLIYSASYRYTGVPDRFSGSGSGTDFTLTISSLQPEDFAVYYCQQH |
| | domain | YITPLTEGAGTKVEIKR |
| | Linker 2 | GSGGS |
| | CD89 TM and | DSIHQDYTTQNLIRMAVAGLVLVALLAILVENWHSHTALNKEASAD |
| | ICD | VAEPSWSQQMCQPGLTFARTPSVCK |
| | (italicized | |
| | section | |
| | denotes the | |
| | transmembrane | |
| | domain | |
| | [TM] | |
| | sequence) | |

Provided herein is a composition comprising a recombinant nucleic acid sequence encoding a CFP comprising a phagocytic or tethering receptor (PR) subunit (e.g., a phagocytic receptor fusion protein (PFP)) comprising: an extracellular domain comprising an antigen binding domain specific to an antigen of a target cell; a transmembrane domain; and an intracellular domain comprising an intracellular signaling domain; and wherein the transmembrane domain and the extracellular domain are operatively linked; and wherein upon binding of the CFP to the antigen on the target cell, the killing or phagocytosis activity of the myeloid cell, such as a neutrophil, monocyte, myeloid dendritic cell (mDC), mast cell or macrophage cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, -fold, 17-fold, 18-fold, 19-fold, 20-fold, 25-fold, 30-fold, 40-fold, 50-fold, 75-fold, or 100-fold compared to a cell not expressing the CFP.

Provided herein is a recombinant nucleic acid sequence encoding a CFP as described in the immediate above paragraph, wherein the intracellular domain comprises at least one innate immune activating intracellular domain, e.g., a pattern recognition receptor intracellular signaling domain, a TLR intracellular signaling domain, an FcR intracellular signaling domain, an intracellular adapter protein signaling domain, or fragments thereof, that is capable of activating innate immune response of the myeloid cell, activating its phagocytic potential, activating inflammatory cytokine and chemokine response, antigen presentation and T cell activation of the myeloid cell that expresses the CFP, and upon contact with its target antigen, e.g., upon engagement of the antigen binding domain with the target antigen.

In some embodiments, the pro-inflammatory signaling domain comprises an intracellular signaling domain derived from TLR3, TLR4, TLR7, TLR 9, TRIF, RIG-1, MYD88, MAL, IRAK1, MDA-5, an IFN-receptor, STING, MAVS, TRIF or TASL intracellular domains, an NLRP family member, NLRP1-14, NOD1, NOD2, Pyrin, AIM2, NLRC4, FCGR3A, FCERIG, IL-1, IL3, IL5, IL-6, IL-12, IL-13, IL-23, TNF, IL-18, IL-23, IL-27, CSF, MCSF, GMCSF, IL17, IP-10, or RANTES.

In some embodiments, the CFP comprises an intracellular signaling domain comprising a sequence derived from protein that activates interferon responsive transcription factors IRF1, IRF2, IRF3, IRF4, IRF5, IRF6, IRF7, IRF8, or IRF9.

In some embodiments, the CFP comprises intracellular signaling domain comprising a sequence derived from an intracellular adaptor protein. In some embodiments, the adaptor protein may comprise a transmembrane that anchors it to a organelle, such as mitochondria, endoplasmic reticulum or a lysosomal compartment. In some embodiments, the intracellular adaptor protein is a cytosolic protein.

A CFP, as described herein may comprise an antigen binding domain of Table 1A, an extracellular/hinge domain of Table 1C, and one or two or more intracellular signaling domain of Table 2. Optionally, a CFP as described herein may comprise a signal peptide sequence of Table 1B.

In some embodiments, a CFP can comprise a sequence with at least 85% sequence identity to a sequence in Table 4. For example, a CFP can comprise a sequence with at least 86%, 87%, 88%, 89%, 90%, 91%, 92%, 93%, 94%, 95%, 96%, 97%, 98%, 99 or 100% sequence identity to a sequence in Table 4. In some embodiments, a CFP further comprises a signal peptide sequence, such as a signal peptide sequence from Table 1B.

In some embodiments, the intracellular signaling domain comprises an intracellular signaling domain derived from a TRIF intracellular domain having an amino acid sequence of any one of SEQ ID NOs: 19-22 or at least 85% sequence identity to any one of SEQ ID NOs: 19-22. In some embodiments, the intracellular signaling domain comprises a sequences that has at least at least 86%, or at least 87%, or at least 88% or at least 89% sequence identity to any one of SEQ ID NOs: 19-22. In some embodiments, the intracellular signaling domain comprises an intracellular signaling domain derived from an TRIF intracellular signaling domain having at least 90% sequence identity to any one of SEQ ID NOs: 19-22. In some embodiments, the intracellular signaling domain comprises a sequences that has at least at least 91%, or at least 92%, or at least 93% or at least 94% sequence identity to any one of SEQ ID NOs: 19-22. In some embodiments, the intracellular signaling domain comprises an intracellular signaling domain derived from an TRIF intracellular signaling domain having at least 95% sequence identity to any one of SEQ ID NOs: 19-22. In some embodiments, the intracellular domain of a CFP comprises an intracellular signaling domain derived from an TRIF intracellular signaling domain having at least 90% sequence identity to any one of SEQ ID NOs: 19-22; wherein the CFP comprises and extracellular binding domain capable of binding a TROP2 molecule on a target cell, a CD89 transmembrane domain, and/or one or more additional intracellular signaling domains.

In some embodiments, an exemplary anti-TROP2-binding CFP described herein comprises an extracellular antigen binding domain having a sequence of any one of SEQ ID NOs: 1-3, or a heavy chain variable domain comprising a CDR3 sequence of GGFGSSYWYFDV, and/or a light chain variable domain comprising a CDR3 sequence of QQHYITPLT and further comprises an intracellular domain of any one of the sequences in Table 2 or Table 3. In some embodiments, an exemplary anti-TROP2-binding CFP described herein comprises a sequence with at least 80-100% sequence identity to any one of SEQ ID NOs: 26-33.

In some embodiments, provided herein is a pharmaceutical product comprising a recombinant mRNA encoding a chimeric antigen receptor comprising: an anti-TROP2 binding scFv extracellular domain; and a CD89 transmembrane domain; wherein the pharmaceutical product is formulated in an aqueous formulation for delivery systemically. In some embodiments, the pharmaceutical composition comprises an scFv comprising a heavy chain and a light chain, the heavy chain comprising a CDR3 having a sequence GGFGSSYWYFDV, and the light chain comprising a CDR3 having a sequence QQHYITPLT. In some embodiments, the heavy chain further comprises a CDR1 sequence of NYGMN, and a CDR2 sequence of WINTYTGEPTYTDDFKG; and the light chain further comprises a CDR1 sequence of KASQDVSIAVA, and a CDR2 sequence of SASYRYT. The pharmaceutical composition described above comprises a CD89 transmembrane domain having a sequence LIRMAVAGLVLVALLAILV.

An exemplary anti-TROP2 second generation chimeric fusion protein (e.g., a drug product) comprising an anti-TROP2 scFv comprising a heavy chain variable domain having a sequence (CDRs highlighted according to Kabat naming convention) QVQLQQSGSELKKPGASVKVSCKASGYTFTNYGMNWVKQAPGQGLKWMGWINTYTG EPTYTDDFKGRFAFSLDTSVSTAYLQISSLKADDTAVYFCARGGFGSSYWYFDVWGQG SLVTVSSG. An exemplary TROP2 ScFv complete construct has the following sequence, MWLQSLLLLGTVACSISQVQLQQSGSELKKPGASVKVSCKASGYTFTNYGMNWVKQ APGQGLKWMGWINTYTGEPTYTDDFKGRFAFSLDTSVSTAYLQISSLKADDTAVYFCA RGGFGSSYWYFDVWGQGSLVTVSSGGGGSGGGGSGGGGSDIQLTQSPSSLSASVGDRV SITCKASQDVSIAVAWYQQKPGKAPKLLIYSASYRYTGVPDRFSGSGSGTDFTLTISSLQ PEDFAVYYCQQHYITPLTFGAGTKVEIKRGSGGSDSIHQDYTTQNLIRMAVAGLVLVALLA ILVENWHSHTALNKEASADVAEPSWSQQMCQPGLTFARTPSVCK. (SEQ ID NO: 41) The bold letters represent the signal peptide sequence. A mature protein may lack this sequence, and one of skill in the art can interpret the protein sequence as expressed without the signal peptide sequence. The underlined regions are the CDR sequence of the scFV that binds to TROP2 in sequence, CDR1, CDR2, CDR3 for the heavy and light chains. The italicize alphabets represent the amino acid sequence of the CD89 TMD.

Chimeric Proteins with TLR Intracellular Domains, TLR Intracellular Signaling Pathways and NF-Kappa B Activation:

In some embodiments, the intracellular signaling domain comprises an intracellular signaling domain derived from a TLR protein. In some embodiments, the CFP designed to comprise an intracellular signaling domain derived from a TLR intracellular signaling domains can activate NF-kappa B upon engagement of the receptor's extracellular domain to its target. In some embodiments, the intracellular domain may comprise an intracellular signaling domain of the endolysosomal TLR, e.g., TLR3, TLR7, TLR8, or TLR9. In some embodiments, the intracellular signaling domain may be derived from a TLR3 protein. In some embodiments, the intracellular signaling domain may be derived from a TLR7, 8, or 9 protein. In some embodiments, the intracellular domain may comprise an intracellular signaling domain of the cell surface TLRs 1, 2, 4, 5, 6, and 10. In some embodiments, the cytoplasmic domain for inflammatory response comprises an intracellular signaling domain of TLR3, TLR4, TLR9, MYD88, TRIF, RIG-1, MDA5, CD40, IFN receptor, NLRP-1, NLRP-2, NLRP-3, NLRP-4, NLRP-5, NLRP-6, NLRP-7, NLRP-8, NLRP-9, NLRP-10, NLRP-11, NLRP-12, NLRP-13, NLRP-14, NOD1, NOD2, Pyrin, AIM2, NLRC4 and/or CD40.

In some embodiments, the phagocytic scavenger receptor (PR) fusion protein (PFP) comprises a pro-inflammatory cytoplasmic domain for activation of IL-1 signaling cascade.

In some embodiments, the cytoplasmic portion of the chimeric receptor (for example, phagocytic receptor (PR) fusion protein (PFP)) comprises a cytoplasmic domain from a toll-like receptor, such as the intracellular signaling domains of toll-like receptor 3 (TLR3), toll-like receptor 4 (TLR4), toll-like receptor 7 (TLR7), toll-like receptor 8 (TLR8), toll-like receptor 9 (TLR9).

In some embodiments, an intracellular domain such as for example, a MyD88, TRIF, TIRAP/MAL, TLR, MAVS, MDA5, STING, RIGI, TASL mentioned herein may be adjusted or modified for pairing or inclusion with another structural domain such as a transmembrane domain. In some embodiments, the transmembrane domain is a CD68 domain. In some embodiments, the transmembrane domain is a CD64 domain. In some embodiments the transmembrane domain is a CD89 domain.

For the purpose of this disclosure, any pathway, signaling intermediate, or activating moiety discussed in the paragraphs above may be considered as activable or functional as it applies, upon induction of the CFP disclosed that comprises a TLR intracellular signaling domain as disclosed herein. Likewise, the CFP disclosed herein may be useful in targeting any of the applicable targets that are described in the pathways discussed. Any pathway or part thereof readily known to one of skill in the art as of date from existing literature that relates to the signaling domain, signaling pathways, signaling intermediates, transcription factors of activated genes is understood to be within the prevue of this disclosure.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the CFP functionally incorporates into a cell membrane of a cell when the CFP is expressed in the cell. In some embodiments, upon binding of the CFP to the antigen of the target cell, the killing activity of a cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, -fold, 17-fold, 18-fold, 19-fold, 20-fold, 25-fold, 30-fold, 40-fold, 50-fold, 75-fold, or 100-fold compared to a cell not expressing the CFP.

### Therapeutic Compositions

In one aspect, TROP2 targeting myeloid cells can be generated ex vivo and a cell therapy product thus generated can be administered to a subject in need thereof. However, generating cell therapy product can be cost expensive and time consuming.

Alternatively, in another aspect, a recombinant polynucleotide can be prepared as a therapeutic component that can be administered to a subject in need thereof, wherein the recombinant polynucleotide is designed for uptake and expression specifically in myeloid cells; wherein expression of the recombinant polynucleotide activates a myeloid cell and induces target cell phagocytosis and killing.

### A. Therapeutic Myeloid Cell Compositions

In some embodiments, the pharmaceutical composition comprises a population of cells comprising therapeutically effective dose of the myeloid cells. In some embodiments, the population of cells: differentiate into effector cells in the subject after administration; infiltrate into a diseased site of the subject after administration or migrate to a diseased site of the subject after administration; and/or have a life-span of at least 5 days in the subject after administration.

In some embodiments, myeloid cells may be further modified or manipulated to develop a therapeutically effective myeloid cells. Isolated cells can be manipulated by expressing a gene or a fragment thereof in the cell, without altering its functional and developmental plasticity, differential potential and cell viability.

In some embodiments, myeloid cells may be further modified or manipulated to develop a therapeutically effective myeloid cells by expressing a non-endogenous polynucleotide into the cell. A non-endogenous polynucleotide may encode for a protein or a peptide. Alternatively, a non-endogenous polypeptide may be a non-coding sequence, such as an inhibitory RNA, or a morpholino.

In some embodiments, myeloid cells may be further modified or manipulated to develop a therapeutically effective myeloid cells by stably altering the genomic sequence of the cell. In some embodiments, the myeloid cell is manipulated by editing the myeloid cell genome using a CRISPR-CAS system. In some embodiments, one or more genes may be edited to silence the gene expression. In some embodiments, the myeloid cell is manipulated to delete a gene. In some embodiments, one or more genes may be edited to enhance the gene expression. In some embodiments, the genetic material is introduced into a myeloid cell in the form of a messenger RNA, wherein the messenger RNA encodes a protein or a peptide, thereby rendering the myeloid cell therapeutically effective. In some embodiments, naked DNA or messenger RNA (mRNA) may be used to introduce the nucleic acid inside the myeloid cell.

B. Therapeutic mRNA Compositions

In some embodiments, DNA or mRNA encoding the chimeric antigen receptor is introduced into the phagocytic cell by a nucleic acid delivery vehicle. In some embodiments, the nucleic acid delivery vehicle may be a nanoparticle encapsulating the nucleic acid cargo. The nanoparticle may be a polymeric nanoparticle. For example, myeloid cells can be targeted using specialized biodegradable polymers, such as PLGA (poly(lactic-co-glycolic) acid and/or polyvinyl alcohol (PVA). In some embodiments, one or more compounds can be selectively incorporated in such polymeric structures to affect the myeloid cell function. In some embodiments, the targeting structures are multilayered, e.g., of one or more PLGA and one or more PVA layers. In some embodiments, the targeting structures are assembled in an order for a layered activity. In some embodiments, the targeted polymeric structures are organized in specific shaped components, such as labile structures that can adhere to a myeloid cell surface and deliver one or more components such as growth factors and cytokines, such as to maintain the myeloid cell in a microenvironment that endows a specific polarization. In some embodiments, the polymeric structures are such that they are not phagocytosed by the myeloid cell, but they can remain adhered on the surface. In some embodiments, the delivery vehicle may comprise a lipid. In some embodiments, the phagocytic cell is in vivo.

In some embodiments the mRNA is single stranded and may be codon optimized. In some embodiments the mRNA may comprise one or more modified or unnatural bases such as 5′-Methylcytosine, or Pseudouridine or methyl pseudouridine. In some embodiments greater than or about 50% uridine (‘U’) residues of the mRNA may be converted to methyl-pseudouridine. In some embodiments, the mRNA may be 50-10,000 bases long. In one aspect the transgene is delivered as an mRNA. The mRNA may comprise greater than about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10,000 bases. In some embodiments, the mRNA may be more than 10,000 bases long. In some embodiments, the mRNA may be about 11,000 bases long. In some embodiments, the mRNA may be about 12,000 bases long. In some embodiments, the mRNA comprises a transgene sequence that encodes a fusion protein. LNP encapsulated DNA or RNA can be used for transfecting a macrophage or can be administered to a subject. In some embodiments, the mRNA is incorporated into an effector myeloid cell population by transient transfection. In some embodiments the transient transfection method comprises electroporation of the mRNA.

In some embodiments a therapeutically effective dose ranges between 1-5,000 micrograms/ml of the mRNA per human subject. In some embodiments, 1-5,000 micrograms/ml of the mRNA may be delivered using a suitable protocol for the methods described above. In some embodiments, 1-2,000 micrograms/ml of the mRNA may be delivered. In some embodiments, 1-1,000 micrograms/ml of the mRNA may be delivered. In some embodiments, 1-1,000 micrograms/ml of the mRNA may be delivered. In some embodiments, 1-500 micrograms/ml of the mRNA may be delivered. In some embodiments, 1-250 micrograms/ml of the mRNA may be delivered. In some embodiments, about 500 micrograms/ml of the mRNA or less may be delivered. In some embodiments, about 250 micrograms/ml of the mRNA or less may be delivered. In some embodiments, about 10 micrograms/ml of the mRNA may be delivered. In some embodiments, about 20 micrograms/ml of the mRNA may be delivered. In some embodiments, about 30 micrograms/ml of the mRNA may be delivered. In some embodiments, about 40 micrograms/ml of the mRNA may be delivered. In some embodiments, about 50 micrograms/ml of the mRNA is used. In some embodiments, about 60 micrograms/ml of the mRNA may be delivered. In some embodiments, about 80 micrograms/ml of the mRNA may be delivered. In some embodiments, about 100 micrograms/ml of the mRNA is used. In some embodiments, about 150 micrograms/ml of the mRNA is use may be delivered d. In some embodiments, about 200 micrograms/ml of the mRNA may be delivered. In some embodiments, 20, 50, 100, 150, 200, 250, 300, 400, 500 or about 1000 micrograms/ml of the mRNA is used as a therapeutically effective dose on a human subject.

mRNA constructs may be thawed on ice and gently pipetted to monocytes and pre-mixed. In some embodiments, the mRNA is electroporated into the cells. Cells following elutriation may be pooled, centrifuged and may be subjected to electroporation with mRNA using MaxCyte ATX system optimized for the said purpose. In some embodiments, optimized electroporation buffer, cell density, and/or mRNA concentration is used for each protocol for each construct.

In some embodiments, a polynucleotide may be introduced into a myeloid cell in the form of a circular RNA (circRNAs). In circular RNAs (circRNAs) the 3′ and 5′ ends are covalently linked. CircRNA may be delivered inside a cell using LNPs.

In some embodiments, a stable integration of transgenes into macrophages and other phagocytic cells may be accomplished via the use of a transposase and transposable elements, in particular, mRNA-encoded transposase. In one embodiment, Long Interspersed Element-1 (L1) RNAs may be contemplated for retrotransposition of the transgene and stable integration into a macrophage or a phagocytic cell. Retrotransposon may be used for stable integration of a recombinant nucleic acid encoding a phagocytic or tethering receptor (PR) fusion protein (PFP).

In some embodiments, the myeloid cell may be modified by expressing a transgene via incorporation of the transgene in a transient expression vector. In some embodiments expression of the transgene may be temporally regulated by a regulator from outside the cell. Examples include the Tet-on Tet-off system, where the expression of the transgene is regulated via presence or absence of tetracycline.

In some embodiments, the myeloid cell may be modified to develop a therapeutically effective cell by contacting the cell with a compound, which compound may be an inhibitor or an activator of a protein or enzyme within the myeloid cell.

In some embodiments, a polynucleotide encoding a chimeric antigen receptor may be introduced into an isolated myeloid cell that is obtained by the method described in the preceding section, where the chimeric antigen receptor upon expression in the myeloid cell augments an innate immune response function of the myeloid cell. In some embodiments, the chimeric antigen receptor expression can direct a myeloid cell to a specific target in vivo or in vitro. In some embodiments, the chimeric antigen receptor may increase the phagocytic potential of the myeloid cell. In some embodiments, the chimeric antigen receptor increases the immunogenicity of the myeloid cell. In some embodiments, the chimeric antigen receptor may increase augment intracellular signaling. In some embodiments, the chimeric antigen receptor may function cooperatively with one or more proteins within the cell. In some embodiments, the chimeric antigen receptor may dimerize or multimerize with a second receptor or transmembrane protein inside the myeloid cell, where the second receptor or transmembrane protein is an endogenous protein. In some embodiments, the cells are cultured ex vivo briefly after thawing or after incorporation of the nucleic acid. In some embodiments, the ex vivo culture is performed in presence of a suitable medium, that may comprise a regulated serum component, e.g., human serum albumin (HSA). In some embodiments, the ex vivo culture and manipulation may be performed in low serum containing media. In some embodiment, the serum is specifically treated for compliment deactivation. In some embodiments, the myeloid cells may be cultured ex vivo as described above, in the presence of M-CSF. In some embodiments, the myeloid cells may be cultured ex vivo as described above, in the presence of GM-CSF. In some embodiments, the myeloid cells may be cultured in the presence of one or more cytokines. In some embodiments, the myeloid cells may be cultured or manipulated ex vivo in the absence of growth factor or cytokines for a period. In some embodiments, the method provided herein comprises isolation or enrichment and manipulation of a myeloid cell in less than 72 hours, 70 hours, 65 hours, 60 hours, 55 hours, 50 hours, 45 hours, 40 hours, or 35 hours, or 30 hours, or 28 hours, or 26 hours or 24 hours. In some embodiments, the myeloid cell may be culture for less than 24 hours, or less than 20 hours or less than 16 hours, or less than 14 hours, or less than 12 hours, or less than 10 hours, or less than 8 hours, or less than 6 hours or less than about 4 hours. The myeloid cell following isolation or enrichment and manipulation may be cultured briefly and frozen till further use. In some embodiments, the myeloid cell is thawed once or at the most twice.

In some embodiments, the therapeutically competent cells are cells that have been electroporated with a recombinant nucleic acid encoding a polypeptide, frozen and thawed, culture stabilized for less than 24 hours, and wherein the cells in the cell population at the time of administration exhibit (i) greater than at least 70% viability, (ii) greater than at least 50% CD14+ and CD16− cells; and/or greater than 50% CD11b+/CD14+/CD16− cells; (iii) less than 5% CD3+ cells, less than 5% CD19+ cells, less than about 10% CD56+ cells, less than about 10% CD42b+ cells (iv) greater than 50% cells express the polypeptide encoded by the electroporated nucleic acid. In some embodiments, the therapeutically competent cells are cells that have been electroporated with a recombinant nucleic acid encoding a polypeptide, culture stabilized for less than 24 hours, frozen and thawed, and wherein the cells in the cell population at the time of administration exhibit (i) greater than at least 70% viability, (ii) greater than at least 50% CD14+ and CD16− cells; and/or greater than 50% CD11b+/CD14+/CD16− cells; (iii) less than 5% CD3+ cells, less than 5% CD19+ cells, less than about 10% CD56+ cells, less than about 10% CD42b+ cells (iv) greater than 50% cells express the polypeptide encoded by the electroporated nucleic acid. In some embodiments, the therapeutically competent cells are cells that have been culture stabilized for less than 24 hours, that have been electroporated with a recombinant nucleic acid encoding a polypeptide, frozen and thawed, and wherein the cells in the cell population at the time of administration exhibit (i) greater than at least 70% viability, (ii) greater than at least 50% CD14+ and CD16− cells; and/or greater than 50% CD11b+/CD14+/CD16− cells; (iii) less than 5% CD3+ cells, less than 5% CD19+ cells, less than about 10% CD56+ cells, less than about 10% CD42b+ cells (iv) greater than 50% cells express the polypeptide encoded by the electroporated nucleic acid. Cells must be pathogen free. In the above embodiments, the therapeutically competent cells may have been frozen and thawed not more than twice, preferably once, and may be administered within 24 hours of thawing, within 18 hours of thawing, within 8 hours of thawing, or within 2 hours of thawing. Cells are tested for quality assurance to meet the standards as described herein in the disclosure prior to administering.

Provided herein are methods for treating cancer in a subject using a pharmaceutical composition comprising engineered phagocytic cells, particularly macrophages, expressing recombinant nucleic acid encoding a phagocytic receptor (PR) fusion protein (PFP), which is specifically designed to target, attack and kill cancer cells. The PFP is also designated as a chimeric antigenic receptor for phagocytosis (CAR-P), and both the terms may be used interchangeably herein. The engineered phagocytic cells are also designated as CAR-P cells in the descriptions herein.

Cancers include, but are not limited to, T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In general, cellular immunotherapy comprises providing the patient a medicament comprising live cells. In some aspects a patient or a subject having cancer, is treated with autologous cells, the method comprising, isolation or enrichment of PBMC-derived macrophages, modifying the macrophages ex vivo to generate highly phagocytic macrophages capable of tumor lysis by introducing into the macrophages a recombinant nucleic acid encoding chimeric antigenic receptor for phagocytosis which is a phagocytic receptor fusion protein (PFP), and administering the modified macrophages into the patient or the subject.

In one aspect, a subject is administered one or more doses of a pharmaceutical composition comprising therapeutic phagocytic cells, wherein the cells are allogeneic. An HLA may be matched for compatibility with the subject, and such that the cells do not lead to graft versus Host Disease, GVHD. A subject arriving at the clinic is HLA typed for determining the HLA antigens expressed by the subject, prior to determining a therapeutic or therapeutic regimen.

In some embodiments a therapeutically effective dose ranges between 10\hat{ }7 cells to 10\hat{ }12 myeloid cells for one infusion. The cell number may vary according to the age, body weight and other subject-related parameters and can be determined by a medical practitioner. In some embodiments, a therapeutically effective dose is about 10\hat{ }7 myeloid cells. In some embodiments, a therapeutically effective dose is about 2×10\hat{ }7 myeloid cells. In some embodiments, a therapeutically effective dose is about 3×10\hat{ }7 myeloid cells. In some embodiments, a therapeutically effective dose is about 4×10\hat{ }7 myeloid cells. In some embodiments, a therapeutically effective dose is about 5×10\hat{ }7 myeloid cells. In some embodiments, a therapeutically effective dose is about 6×10\hat{ }7 myeloid cells. In some embodiments, a therapeutically effective dose is about 7×10\hat{ }7 myeloid cells. In some embodiments, a therapeutically effective dose is about 8×10\hat{ }7 myeloid cells. In some embodiments, a therapeutically effective dose is about 9×10\hat{ }7 myeloid cells. In some embodiments, a therapeutically effective dose is about 10\hat{ }8 myeloid cells. In some embodiments, a therapeutically effective dose is about 2×10\hat{ }8 myeloid cells. In some embodiments, a therapeutically effective dose is about 3×10\hat{ }8 myeloid cells. In some embodiments, a therapeutically effective dose is about 4×10\hat{ }8 myeloid cells. In some embodiments, a therapeutically effective dose is about 5×10\hat{ }8 myeloid cells. In some embodiments, a therapeutically effective dose is about 6×10\hat{ }8 myeloid cells. In some embodiments, a therapeutically effective dose is about 7×10\hat{ }8 myeloid cells. In some embodiments, a therapeutically effective dose is about 8×10\hat{ }8 myeloid cells. In some embodiments, a therapeutically effective dose is about 9×10\hat{ }8 myeloid cells. In some embodiments, a therapeutically effective dose is about 10\hat{ }9 myeloid cells. In some embodiments, a therapeutically effective dose is about 2×10\hat{ }9 myeloid cells. In some embodiments, a therapeutically effective dose is about 3×10\hat{ }9 myeloid cells. In some embodiments, a therapeutically effective dose is about 4×10\hat{ }9 myeloid cells.

In some embodiments, a therapeutically effective dose is about 5×10\hat{ }9 myeloid cells. In some embodiments, a therapeutically effective dose is about 6×10\hat{ }9 myeloid cells. In some embodiments, a therapeutically effective dose is about 7×10\hat{ }9 myeloid cells. In some embodiments, a therapeutically effective dose is about 8×10\hat{ }9 myeloid cells. In some embodiments, a therapeutically effective dose is about 9×10\hat{ }9 myeloid cells. In some embodiments, a therapeutically effective dose is about 10\hat{ }10 myeloid cells. In some embodiments, a therapeutically effective dose is about 5×10\hat{ }10 myeloid cells. In some embodiments a therapeutically effective dose is about 10\hat{ }11 myeloid cells. In some embodiments a therapeutically effective dose is about 5×10\hat{ }11 myeloid cells. In some embodiments a therapeutically effective dose is about 10\hat{ }12 myeloid cells.

### Methods for Generation of Novel Chimeric Receptors Fusion Proteins (CFP) Constructs

In one aspect, provided herein is a method for generating novel chimeric receptor proteins, including, for example, identification of novel domains that can be useful in augmenting a myeloid cell function such that when the fusion receptor is expressed in a myeloid cell, it functions as an effector myeloid cell of the specifications described herein. Generation of fusion proteins as described herein can be performed using well known molecular cloning techniques, and the sequences can be verified after generating of the recombinant nucleic acid.

Preparation of Recombinant Nucleic Acid Encoding A Chimeric Antigen Receptor: Recombinant nucleic acid constructs are prepared that encode chimeric antigen receptor (CAR) designed for expression in a myeloid cell and are incorporated in plasmid vectors for amplification and/or testing expression in an eukaryotic cell. The recombinant CARs are constructed using molecular cloning techniques known in the art. A recombinant CAR protein comprises an intracellular domain, a transmembrane domain and an extracellular domain. Each domain or subsection of a domain can be encoded by a nucleic acid sequence that is generated by PCR from heterologous source sequences, and pieced together by cloning individually into the vector, or ligated into a longer nucleic acid that is then inserted into the multi-cloning sites of a suitable plasmid or vector with appropriate promoter and 3′-regulatory elements for amplification. Briefly, an exemplary CAR is prepared by incorporating a nucleic sequence encoding one or more signaling domains, (e.g., a PI3Kinase recruiting domain), a nucleic acid sequence encoding the CD8 hinge and transmembrane domain, a nucleic acid sequence encoding an extracellular domain, having a sequence encoding a target antigen binding scFv at the extracellular end. Certain constructs include a FLAG peptide sequence at the extracellular end designed such that it does not pose hindrance to the scFv binding to its target antigen. These components are ligated together into a sequence that encode a fully functional transmembrane CAR. The nucleic acid subunits encoding individual domains of the recombinant protein is designed to include intervening short flexible linker sequences between two domains. The construct is ligated in a plasmid having a promoter and 3′ stabilizing structural units. In one variation, the construct is placed within an Alu retrotransposon element that encodes ORF2p and has the respective 5′- and 3′-UTR sequences, a CMV promoter. The plasmid is amplified in E. coli, validated by sequencing or stored in (−) 80° C.

### Phagocytosis Assay:

Antigen-linked silica or polysterene beads ranging in diameters 1 nm, 5 nm or 10 nm were used for a screen of macrophages. Inert beads can be coated in a supported lipid bilayer and the antigens can be ligated to the lipid bilayer. J774 macrophage cell lines can be prepared, each cell line expressing a cloned recombinant plasma membrane protein. The recombinant plasma membrane protein may also express a fluorescent tag. The cell lines can be maintained and propagated in complete RPMI media with heat inactivated serum and antibiotics (Penicillin/Streptomycin). On the day of the assay, cells can be plated at a density of 1×10\hat{ }6 cells/ml per well in 6 well plates or in a relative proportion in 12 or 24 well plates, and incubated for 2-6 hours. The cells can be then washed once in Phosphate Buffer Saline, and the beads can be added in serum depleted or complement depleted nutrient media. Cells can be visualized by light microscopy at 30 minutes and 2 hours after addition of the beads. Immunofluorescence reaction may be performed using tagged antibody, and fluorescent confocal microscopy is used to detect the interaction and co-localization of cellular proteins at engulfment. Confidence levels can be determined by Kruskal-Wallis test with Dunn's multiple comparison correction.

In some examples, dye loaded tumor cells can be fed to macrophage cell lines and phagocytosis is assessed by microscopy.

### Cytokine Production:

Macrophage cell lines can be cultured as described above. In one assay, each J774 cell line expressing a plasma membrane protein is plated in multi-wells and challenged with antigen-linked beads and cytokine production was assayed by collecting the supernatants at 4 hours and 24 hours. Cytokines can be assayed from the supernatant by ELISA. In another fraction, cells can be collected at 4 and 24 hours after incubation with the beads and flow cytometry is performed for detection of cytokines. In each case, multiple cytokines can be assayed in a multiplex format, which can be selected from: IL-1α, IL-1β, IL-6, IL-12, IL-23, TNF-α, GMCSF, CXCL1, CXCL3, CXCL9, CXCL-10, MIP1-α and MIP-2. Macrophage inflammatory cytokine array kit (R&D Systems) is used.

Intracellular signaling pathway for inflammatory gene and cytokine activation can be identified by western blot analysis for phosphorylation of MAP kinases, JNK, Akt signaling pathway, Interferon activation pathway including phosphorylation and activation of STAT-1.

### Functional Assays

### Inflammasome Activation Assay:

Activation of NLRP3 inflammasome is assayed by ELISA detection of increased IL-1 production and detection caspase-1 activation by western blot, detecting cleavage of procaspase to generate the shorter caspase. In a microwell plate multiplex setting, Caspase-Glo (Promega Corporation) is used for faster readout of Caspase 1 activation.

iNOS Activation Assay:

Activation of the oxidative burst potential can be measured by iNOS activation and NO production using a fluorimetric assay NOS activity assay kit (AbCAM).

### Cancer Cell Killing Assay:

Raji B cells can be used as cancer antigen presenting cells. Raji cells can be incubated with whole cell crude extract of cancer cells, and co-incubated with J774 macrophage cell lines. The macrophages can destroy the cells after 1 hour of infection, which can be detected by microscopy or detected by cell death assay.

Method of Manufacturing Myeloid Cells from a Subject
Myeloid Cell Isolation from PBMCs:

Peripheral blood mononuclear cells can be separated from normal donor buffy coats by density centrifugation using Histopaque 1077 (Sigma). After washing, CD14+ monocytes can be isolated from the mononuclear cell fraction using CliniMACS GMP grade CD14 microbeads and LS separation magnetic columns (Miltenyi Biotec). Briefly, cells can be resuspended to appropriate concentration in PEA buffer (phosphate-buffered saline [PBS] plus 2.5 mmol/L ethylenediaminetetraacetic acid [EDTA] and human serum albumin [0.5% final volume of Alburex 20%, Octopharma]), incubated with CliniMACS CD14 beads per manufacturer's instructions, then washed and passed through a magnetized LS column. After washing, the purified monocytes can be eluted from the demagnetized column, washed and re-suspended in relevant medium for culture. Isolation of CD14+ cells from leukapheresis: PBMCs can be collected by leukapheresis from cirrhotic donors who gave informed consent to participate in the study. Leukapheresis of peripheral blood for mononuclear cells (MNCs) is carried out using an Optia apheresis system by sterile collection. A standard collection program for MNC is used, processing 2.5 blood volumes. Isolation of CD14 cells is carried out using a GMP-compliant functionally closed system (CliniMACS Prodigy system, Miltenyi Biotec). Briefly, the leukapheresis product is sampled for cell count and an aliquot taken for pre-separation flow cytometry. The percentage of monocytes (CD14+) and absolute cell number can be determined, and, if required, the volume is adjusted to meet the required criteria for selection (≤20×10⁹ total white blood cells; <400×10⁶ white blood cells/mL; ≤3.5×10⁹ CD14 cells, volume 50-300 mL). CD14 cell isolation and separation is carried out using the CliniMACS Prodigy with CliniMACS CD14 microbeads (medical device class III), TS510 tubing set and LP-14 program. At the end of the process, the selected CD14+ positive monocytes can be washed in PBS/EDTA buffer (CliniMACS buffer, Miltenyi) containing pharmaceutical grade 0.5% human albumin (Alburex), then re-suspended in TexMACS (or comparator) medium for culture.

### Cell Count and Purity:

Cell counts of total MNCs and isolated monocyte fractions can be performed using a Sysmex XP-300 automated analyzer (Sysmex). Assessment of macrophage numbers is carried out by flow cytometry with TruCount tubes (Becton Dickinson) to determine absolute cell number, as the Sysmex consistently underestimated the number of monocytes. The purity of the separation is assessed using flow cytometry (FACSCanto II, BD Biosciences) with a panel of antibodies against human leukocytes (CD45-VioBlue, CD15-FITC, CD14-PE, CD16-APC), and product quality is assessed by determining the amount of neutrophil contamination (CD45int, CD15pos).

Cell Culture—Development of Cultures with Healthy Donor Samples

Optimal culture medium for macrophage differentiation is investigated, and three candidates can be tested using for the cell product. In addition, the effect of monocyte cryopreservation on deriving myeloid cells and macrophages for therapeutic use is examined. Functional assays can be conducted to quantify the phagocytic capacity of myeloid cells and macrophages and their capacity for further polarization, and phagocytic potential as described elsewhere in the disclosure.

Full-Scale Process Validation with Subject Samples

Monocytes cultured from leukapheresis from Prodigy isolation can be cultured at 2×10⁶ monocytes per cm² and per mL in culture bags (MACS GMP differentiation bags, Miltenyi) with GMP-grade TexMACS (Miltenyi) and 100 ng/mL M-CSF. Monocytes can be cultured with 100 ng/ml GMP-compliant recombinant human M-CSF (R&D Systems). Cells can be cultured in a humidified atmosphere at 37° C., with 5% CO₂ for 7 days. A 50% volume media replenishment is carried out twice during culture (days 2 and 4) with 50% of the culture medium removed, then fed with fresh medium supplemented with 200 ng/ml M-CSF (to restore a final concentration of 100 ng/ml).

### Cell Harvesting:

For normal donor-derived macrophages, cells can be removed from the wells at day 7 using Cell Dissociation Buffer (Gibco, Thermo Fisher) and a pastette. Cells can be resuspended in PEA buffer and counted, then approximately 1×10⁶ cells per test can be stained for flow cytometry. Leukapheresis-derived macrophages can be removed from the culture bags at day 7 using PBS/EDTA buffer (CliniMACS buffer, Miltenyi) containing pharmaceutical grade 0.5% human albumin from serum (HAS; Alburex). Harvested cells can be resuspended in excipient composed of two licensed products: 0.9% saline for infusion (Baxter) with 0.5% human albumin (Alburex).

### Flow Cytometry Characterization:

Monocyte and macrophage cell surface marker expression can be analyzed using either a FACSCanto II (BD Biosciences) or MACSQuant 10 (Miltenyi) flow cytometer. Typically, approximately 20,000 events can be acquired for each sample. Cell surface expression of leukocyte markers in freshly isolated and day 7 matured cells is carried out by incubating cells with specific antibodies (final dilution 1:100). Cells are incubated for 5 min with FcR block (Miltenyi) then incubated at 4° C. for 20 min with antibody cocktails. Cells can be washed in PEA, and dead cell exclusion dye DRAQ7 (BioLegend) is added at 1:100. Cells can be stained for a range of surface markers as follows: CD45-VioBlue, CD14-PE or CD14-PerCP-Vio700, CD163-FITC, CD169-PE and CD16-APC (all Miltenyi), CCR2-BV421, CD206-FITC, CXCR4-PE and CD115-APC (all BioLegend), and 25F9-APC and CD115-APC (eBioscience). Both monocytes and macrophages can be gated to exclude debris, doublets and dead cells using forward and side scatter and DRAQ7 dead cell discriminator (BioLegend) and analyzed using FlowJo softwcan be (Tree Star). From the initial detailed phenotyping, a panel is developed as Release Criteria (CD45-VB/CD206-FITC/CD14-PE/25F9 APC/DRAQ7) that defined the development of a functional macrophage from monocytes. Macrophages can be determined as having mean fluorescence intensity (MFI) five times higher than the level on day 0 monocytes for both 25F9 and CD206. A second panel is developed which assessed other markers as part of an Extended Panel, composed of CCR2-BV421/CD163-FITC/CD169-PE/CD14-PerCP-Vio700/CD16-APC/DRAQ7), but is not used as part of the Release Criteria for the cell product.

Monocytes and macrophages can be isolated from withdrawing a buffy coat layer formed in a sucrose gradient centrifugation sample of isolated peripheral blood cells. CD14 cells can be tested for phagocytic uptake using pHRodo beads, which fluoresce only when taken into acidic endosomes. Briefly, monocytes or macrophages can be cultured with 1-2 μL of pHRodo Escherichia coli bioparticles (Life Technologies, Thermo Fisher) for 1 h, then the medium is taken off and cells are washed to remove non-phagocytosed particles. Phagocytosis is assessed using an EVOS microscope (Thermo Fisher), images captured and cellular uptake of beads quantified using ImageJ software (NIH). The capacity to polarize toward defined differentiated macrophages is examined by treating day 7 macrophages with IFNγ (50 ng/ml) or IL-4 (20 ng/mL) for 48 h to induce polarization to M1 or M2 phenotype (or M[IFNγ] versus M[IL-4], respectively). After 48 h, the cells can be visualized by EVOS bright-field microscopy, then harvested and phenotyped as before. Further analysis is performed on the cytokine and growth factor secretion profile of macrophages after generation and in response to inflammatory stimuli. Macrophages can be generated from healthy donor buffy coats as before, and either left untreated or stimulated with TNFα (50 ng/mL, Peprotech) and polyinosinic: polycytidylic acid (poly I:C, a viral homolog which binds TLR3, 1 g/mL, Sigma) to mimic the conditions present in the inflamed liver, or lipopolysaccharide (LPS, 100 ng/mL, Sigma) plus IFNγ (50 IU/mL, Peprotech) to produce a maximal macrophage activation. Day 7 macrophages can be incubated overnight and supernatants collected and spun down to remove debris, then stored at −80° C. until testing. Secretome analysis is performed using a 27-plex human cytokine kit and a 9-plex matrix metalloprotease kit run on a Magpix multiplex enzyme linked immunoassay plate reader (BioRad).

### Product Stability:

Various excipients can be tested during process development including PBS/EDTA buffer; PBS/EDTA buffer with 0.5% HAS (Alburex), 0.9% saline alone or saline with 0.5% HAS. The 0.9% saline (Baxter) with 0.5% HAS excipient is found to maintain optimal cell viability and phenotype (data not shown). The stability of the macrophages from cirrhotic donors after harvest is investigated in three process optimization runs, and a more limited range of time points assessed in the process validation runs (n=3). After harvest and re-suspension in excipient (0.9% saline for infusion, 0.5% human serum albumin), the bags can be stored at ambient temperature (21-22° C.) and samples taken at 0, 2, 4, 6, 8, 12, 24, 30 and 48 h postharvest. The release criteria antibody panel is run on each sample, and viability and mean fold change from day 0 is measured from geometric MFI of 25F9 and CD206. For mRNA product all excipients and equipment must be satisfactorily RNase treated and free of RNase.

### Statistical Analysis:

Results can be expressed as mean±SD. The statistical significance of differences is assessed where possible with the unpaired two-tailed t-test using GraphPad Prism 6. Results can be considered statistically significant when the P value is <0.05.

Also provided herein is a cell comprising a composition described herein, a vector described herein or a polypeptide described herein. In some embodiments, the cell is a phagocytic cell. In some embodiments, the cell is a stem cell derived cell, a myeloid cell, a macrophage, a dendritic cell, a lymphocyte, a mast cell, a monocyte, a neutrophil, a microglia, or an astrocyte. In some embodiments, the cell is an autologous cell. In some embodiments, the cell is an allogeneic cell. In some embodiments, the cell is an M1 cell. In some embodiments, the cell is an M2 cell. In some embodiments, the cell is an M1 macrophage cell. In some embodiments, the cell is an M2 macrophage cell. In some embodiments, the cell is an M1 myeloid cell. In some embodiments, the cell is an M2 myeloid cell.

Also provided herein is a method of treating a disease in a subject in need thereof comprising administering to the subject a pharmaceutical composition described herein. In some embodiments, the disease is cancer. In some embodiments, the cancer is a solid cancer. In some embodiments, the solid cancer is selected from the group consisting of ovarian cancer, suitable cancers include ovarian cancer, renal cancer, breast cancer, prostate cancer, liver cancer, brain cancer, lymphoma, leukemia, skin cancer, pancreatic cancer, colorectal cancer, lung cancer. In some embodiments, the cancer is a liquid cancer. In some embodiments, the liquid cancer is leukemia or a lymphoma. In some embodiments, the liquid cancer is a T cell lymphoma. In some embodiments, the disease is a T cell malignancy.

In some embodiments, administering comprises infusing or injecting. In some embodiments, administering comprises administering directly to the solid cancer. In some embodiments, administering comprises a circRNA-based delivery procedure, anon-particle encapsulated mRNA-based delivery procedure, an mRNA delivery procedure, particle-based delivery procedure, liposome-based delivery procedure, or an exosome-based delivery procedure.

In some embodiments, a CD4+ T cell response or a CD8+ T cell response is elicited in the subject.

Provided herein is a method for administering a therapeutic comprising any one of the compositions described above. In some embodiments, the therapeutic is administered via a parenteral administration route.

In some embodiments the drug substance is an mRNA. In some embodiments, the drug product is an aqueous or organic solution comprising the mRNA. In some embodiments, the drug substance is in an aqueous excipient. In some embodiments, the drug product may comprise a lipid component. In some embodiments, the drug product may comprise a delivery nanoparticle encapsulating the mRNA. In some embodiments, the mRNA is delivered systemically in a subject in need thereof, the subject being a human. The concentration and total volume are determined based several factors, for example, on age, weight, among other things. In some embodiments, a pharmaceutical composition, e.g., a drug product is delivered systemically, for example, via intravenous, cutaneous, subcutaneous, respiratory, oral, intramuscular or other routes.

In some embodiments, the therapeutic is administered via intravenous administration route. In some embodiments, the therapeutic is administered via subcutaneous administration route. In some embodiments, the therapeutic is administered via intramuscular administration route.

Also provided herein is a method of preparing a pharmaceutical composition comprising the one or more recombinant nucleic acids described herein and a lipid in an aqueous composition described herein. In some embodiments, the composition comprises a vector described herein. In some embodiments, the lipid comprises forming a lipid nanoparticle.

### Pharmaceutical Compositions

In some embodiments, a pharmaceutical composition comprises a composition provided herein and a pharmaceutically acceptable excipient. In some embodiments, the pharmaceutical composition is an aqueous formulation. In some embodiments, the pharmaceutical composition is an aqueous pharmaceutical formulation which has a stable shelf life of at least 6 months. In some embodiments, the aqueous pharmaceutical formulation is lyophilizeable. In some embodiments, the lyophilized composition of the aqueous pharmaceutical formulation further comprises a lyoprotectant. In some embodiments, the lyoprotectant is sucrose. In some embodiments, the lyophilized composition has a stable shelf life of at least 6 months.

In some embodiments, a pharmaceutical composition is provided herein comprising a dose of a recombinant mRNA. In some embodiments, the engineered RNA (may interchangeably termed recombinant mRNA) encodes a TROP2 binding CFP. In some embodiments, a pharmaceutical composition is provided herein comprising a dose of a engineered RNA (engineered mRNA) encoding an anti-TROP2 CFP. In some embodiments, a pharmaceutical composition provided herein comprises a dose of a recombinant mRNA, wherein the recombinant mRNA comprises a sequence encoding a chimeric fusion protein (CFP), the CFP comprising (i) an extracellular domain comprising a TROP2 binding domain; (ii) a transmembrane domain; and (iii) an intracellular domain. In some embodiments, the transmembrane domain comprises a CD89 transmembrane domain. In some embodiments, the transmembrane domain is operatively linked to the extracellular domain. In some embodiments, the intracellular domain comprises a CD89 intracellular domain. Unless otherwise mentioned, every dose measurement indicates the amount of RNA in mgs in the pharmaceutical composition. The RNA is mRNA, which is an engineered mRNA. For example, 1 mg/kg of the pharmaceutical product (DP), e.g., anti-TROP2-CD89 RNA: LNP contains 1 mg of the RNA for every kilograms of body weight of the subject administered a single dose of it. A reported 1.0 mg/mL is 1.0 mg of mRNA/mL of mRNA-LNP product. For example, a dose of 0.10 mg/kg is “0.10 mg of mRNA/kg of patient weight.”

In some embodiments, a pharmaceutical composition comprises a dose of an engineered RNA, wherein the engineered RNA (e.g., engineered mRNA) comprises a sequence encoding a chimeric fusion protein (CFP), the CFP comprising: (i) an extracellular domain comprising an anti-TROP2 binding domain; and (ii) a CD89 transmembrane domain operatively linked to the extracellular domain.

In some embodiments, the dose is from about 0.0005 mg/kg to about 0.5 mg/kg of a engineered mRNA.

In some embodiments, the dose is from about 0.0005 to about 0.001, about 0.001 to about 0.005, about 0.005 to about 0.01, about 0.01 to about 0.05, about 0.05 to about 0.1, or about 0.1 to about 0.5 mg/kg of the recombinant mRNA. In some embodiments, the dose is from about 0.001 to about 0.0015, about 0.0015 to about 0.002, about 0.002 to about 0.0025, about 0.0025 to about 0.003, about 0.003 to about 0.0035, about 0.0035 to about 0.004, about 0.004 to about 0.0045, about 0.0045 to about 0.005, about 0.005 to about 0.0055, about 0.0055 to about 0.006, about 0.006 to about 0.0065, about 0.0065 to about 0.007, about 0.007 to about 0.0075, about 0.0075 to about 0.008, about 0.008 to about 0.0085, about 0.0085 to about 0.009, about 0.009 to about 0.0095, or about 0.0095 to about 0.01 mg/kg of the engineered mRNA. In some embodiments, the dose is from about 0.01 to about 0.015, about 0.015 to about 0.02, about 0.02 to about 0.025, about 0.025 to about 0.03, about 0.03 to about 0.035, about 0.035 to about 0.04, about 0.04 to about 0.045, about 0.045 to about 0.05, about 0.05 to about 0.055, about 0.055 to about 0.06, about 0.06 to about 0.065, about 0.065 to about 0.07, about 0.07 to about 0.075, about 0.075 to about 0.08, about 0.08 to about 0.085, about 0.085 to about 0.09, about 0.09 to about 0.095, or about 0.095 to about 0.1 mg/kg of the engineered mRNA.

In some embodiments, the dose is about 0.001, about 0.0015, about 0.002, about 0.0025, about 0.003, about 0.0035, about 0.004, about 0.0045, about 0.005, about 0.0055, about 0.006, about 0.0065, about 0.007, about 0.0075, about 0.008, about 0.0085, about 0.009, about 0.0095, or about 0.01 mg/kg of the recombinant mRNA. In some embodiments, the dose is about 0.01, about 0.015, about 0.02, about 0.025, about 0.03, about 0.035, about 0.04, about 0.045, about 0.05, about 0.055, about 0.06, about 0.065, about 0.07, about 0.075, about 0.08, about 0.085, about 0.09, about 0.095, or about 0.1 mg/kg of the engineered mRNA.

In some embodiments, the dose is at least about 0.001, at least about 0.0015, at least about 0.002, at least about 0.0025, at least about 0.003, at least about 0.0035, at least about 0.004, at least about 0.0045, at least about 0.005, at least about 0.0055, at least about 0.006, at least about 0.0065, at least about 0.007, at least about 0.0075, at least about 0.008, at least about 0.0085, at least about 0.009, at least about 0.0095, or at least about 0.01 mg/kg of the recombinant mRNA. In some embodiments, the dose is at least about 0.01, at least about 0.015, at least about 0.02, at least about 0.025, at least about 0.03, at least about 0.035, at least about 0.04, at least about 0.045, at least about 0.05, at least about 0.055, at least about 0.06, at least about 0.065, at least about 0.07, at least about 0.075, at least about 0.08, at least about 0.085, at least about 0.09, at least about 0.095, or at least about 0.1 mg/kg of the engineered mRNA.

In some embodiments, the concentration of the recombinant mRNA in the pharmaceutical composition is between about 1.5 to about 0.5 mg/mL or between about 1.3 to about 0.7 mg/mL when stored in a container. In some embodiments, the container is a single-use or multi-use vial.

In some embodiments, the pharmaceutical composition is formulated for intravenous delivery.

### Methods

Provided herein are methods for in vivo delivery of innate immune cell chimeric antigen receptors (CARs) delivered as polynucleotides such as ribonucleic acid (e.g., mRNA) constructs. In some embodiments, a lipid nanoparticle (LNP) encapsulates an engineered mRNA construct for in vivo delivery. In some embodiments, the mRNA construct is delivered systemically via LNP. In some embodiments, systemic delivery of an mRNA encoding a CAR directed to tumor-associated antigens results in the generation of CAR myeloid cells in vivo, and in antigen-specific activation and tumor cell killing.

In some embodiments, the engineered mRNA is expressed predominantly in myeloid cells in vivo. In some embodiments, the engineered mRNA is expressed in vascularized tissues of the subject after infusion. In some embodiments, the expression of a sequence encoded by the engineered mRNA is detectable in a cell within the subject for about 72 hours post infusion. In some embodiments, the administration of the pharmaceutical composition does not generate a cytokine response.

Provided herein is a method of treating a cancer in a subject in need thereof, the method comprising administering to the subject an effective amount of a pharmaceutical composition provided herein. In some embodiments, the subject is human. In some embodiments, administering comprises administering to the subject intravenously.

In some embodiments, the subject is pretreated with medicines, drugs, immunomodulators or immunosuppressive agents to prepare for the therapy described herein. For example, the subject may be administered a specific dose of fludarabine and/or cycloheximide at least once prior to commencement with the therapeutic agent described herein, e.g., an engineered RNA comprising a sequence encoding a CAR, delivered by LNP. In some embodiments, the subject is administered more than once, the dose or fludarabine and/or cycloheximide at a specific interval prior to the first or a subsequent administration of the therapeutic agent comprising an engineered RNA comprising a sequence encoding a CAR. In some embodiments, the subject is administered an anti-inflammatory drug, an anti-emetic drug, an anti-pyretic drug, an antihistaminic drug, an H2 blocker (H2 receptor antagonist), a beta adrenergic blocker, or an anti-cancer drug, as is determined by a medically trained person responsible for the subject, or as directed by the protocol. In some embodiment, a subject may be administered a cytokine. In some embodiments, the cytokine may be administered at least once, at least twice, at least three times, at least four times, at least five times, at least six times, at least seven times or more as is necessary and is determined by the medically trained personnel. In some embodiments, the cytokine is a colony stimulating factor (CSF). In some embodiments the cytokine is a granulocyte colony stimulating factor (GCSF, or CSF3). In some embodiments the cytokine is a granulocyte colony stimulating factor (MCSF, or CSF1). In some embodiments the cytokine is a granulocyte macrophage colony stimulating factor (GMCSF). In some embodiments, the cytokine is IL1. In some embodiments, the cytokine is IL2. In some embodiments, the cytokine is TNF-alpha. In some embodiments, the cytokine is interferon gamma (IFN-γ).

In some embodiments, the effective amount of the pharmaceutical composition ranges from 0.01 mg/kg/dose to 3.0 mg/kg/dose, wherein, milligram (mg) refers to the amount in weight of the engineered mRNA (encoding the CAR) in a unit dose of the drug, administered per kilogram of body weight of a subject. A dose usually refers to an amount (absolute or relative) that is administered to a subject in one single administration. An administration may be, for example an injection, or for example an infusion. An absolute value of a dose may be represented in the example: x mg, which indicates that a total of x mg is administered to the subject in one administration. A relative value of a dose may be represented in the example: x mg/kg, which indicates that x mg is administered to the subject per kg of her/his/its body weight in one administration, for example, if the subject weighs 80 kg, the subject receives 80 times mg of the drug in one administration. In some embodiments, the effective amount of the pharmaceutical composition ranges from 0.05 mg/kg/dose to 2.5 mg/kg/dose. In some embodiments, the effective amount of the pharmaceutical composition ranges from 0.1 mg/kg/dose to 1.0 mg/kg/dose.

In some embodiments, the effective amount of the pharmaceutical composition comprises about 0.1, 0.15, 0.2, 0.22, 0.25, 0.275, 0.3, 0.32, 0.33, 0.34, 0.35, 0.36, 0.38, 0.4, 0.45, 0.475, 0.5, 0.525, 0.55, 0.575, 0.6, 0.7, 0.8, 0.9 or 1.0 mg/kg/dose.

In some embodiments, the effective amount of the pharmaceutical composition is administered in a 60-minute intravenous (IV) infusion per dose.

In some embodiments, the effective amount of the pharmaceutical composition is administered for 2, 3, 4, 5, 6, 7, 8, 9, 10 or more IV infusion doses.

In some embodiments, the effective amount of the pharmaceutical composition is administered at an interval of once in a week.

In some embodiments, the effective amount of the pharmaceutical composition is administered at an interval of once every 10 days.

In some embodiments, the effective amount of the pharmaceutical composition is administered at an interval of once every 2 weeks.

In some embodiments, the effective amount of the pharmaceutical composition is administered for at least 5 doses.

In some embodiments, the effective amount of the pharmaceutical composition is administered at 1 mg/kg/dose at days 1, 8, 15, 29, 36 and 43.

Provided herein is a method of treating a cancer in a subject in need thereof, the method comprising administering to the subject a pharmaceutical composition comprising a recombinant mRNA, wherein the recombinant mRNA comprises a sequence encoding a chimeric fusion protein (CFP), the CFP comprising: an extracellular domain comprising a TROP2 binding domain; and a CD89 transmembrane domain operatively linked to the extracellular domain. In some embodiments, the method comprises administering the pharmaceutical composition to the subject for at least one treatment cycle. In some embodiments, the method comprises administering the pharmaceutical composition to the subject for at least two treatment cycles. In some embodiments, the method comprises administering the pharmaceutical composition to the subject for at least three treatment cycles. In some embodiments, the method comprises administering the pharmaceutical composition to the subject for at least four treatment cycles. In some embodiments, the method comprises administering the pharmaceutical composition to the subject for at least five treatment cycles. In some embodiments, the method comprises administering the pharmaceutical composition to the subject for at least six treatment cycles.

In some embodiments, the pharmaceutical composition is administered to the subject about once a week (QW), about once every 2 weeks (Q2W), about once every 3 weeks (Q3W), about once every 4 weeks (Q4W), about once every 5 weeks (Q5W), about once every 6 weeks (Q6W), about once every 7 weeks (Q7W), about once every 8 weeks (Q8W), about once every 9 weeks (Q9W), about once every 10 weeks (Q10W), about once every 11 weeks (Q11W), or about once every 12 weeks (Q12W) for each of the treatment cycle. In some embodiments, the method comprises administering the pharmaceutical composition to the subject QW or Q2W or Q4W for each of the treatment cycle. In some embodiments, the method comprises administering the pharmaceutical composition to the subject with the same frequency in each of the treatment cycle. In some embodiments, the method comprises administering the pharmaceutical composition to the subject with different frequencies for at least two of the treatment cycles. In some embodiments, the method comprises administering the pharmaceutical composition to the subject QW or Q2W in the first treatment cycle.

In some embodiments, the method comprises administering the pharmaceutical composition to the subject at least 1, at least 2, at least 3, at least 4, at least 5, at least 6, at least 7, at least 8, at least 9, at least 10, at least 11, or at least 12 times in the first treatment cycle. In some embodiments, the method comprises administering the pharmaceutical composition to the subject at least 1 to at least 3, at least 3 to at least 6, at least 6 to at least 9, or at least 9 to at least 12 times in the first treatment cycle. In some embodiments, the method comprises administering the pharmaceutical composition to the subject at most 1, at most 2, at most 3, at most 4, at most 5, at most 6, at most 7, at most 8, at most 9, at most 10, at most 11, or at most 12 times in the first treatment cycle. In some embodiments, the method comprises administering the pharmaceutical composition to the subject at most 1 to at most 3, at most 3 to at most 6, at most 6 to at most 9, or at most 9 to at most 12 times. In some embodiments, the first cycle comprises administering the pharmaceutical composition to the subject between 1 to 12 times in the first treatment cycle.

In some embodiments, the method comprises administering the pharmaceutical composition to the subject between about 1 to about 3, about 3 to about 6, about 6 to about 9, or about 9 to about 12 times in the first treatment cycle.

In some embodiments, the method comprises administering the pharmaceutical composition to the subject 3 times in the first treatment cycle.

In some embodiments, a second cycle follows the first cycle. In some embodiments, the method further comprises administering the pharmaceutical composition to the subject during the second cycle.

In some embodiments, the pharmaceutical composition is administered to the subject about QW, about Q2W, about Q3W, about Q4W, about Q5W, about Q6W, about Q7W, about Q8W, about Q9W, about Q10W, about Q11W, or about Q12W during the second cycle. In some embodiments, the method comprises administering the pharmaceutical composition to the subject Q4W during the second cycle.

In some embodiments, the method comprises administering the pharmaceutical composition to the subject at least 1, at least 2, at least 3, at least 4, at least 5, at least 6, at least 7, at least 8, at least 9, at least 10, at least 11, or at least 12 times in the second cycle. In some embodiments, the method comprises administering the pharmaceutical composition to the subject at least 1 to at least 3, at least 3 to at least 6, at least 6 to at least 9, or at least 9 to at least 12 times in the second cycle. In some embodiments, the method comprises administering the pharmaceutical composition to the subject at most 1, at most 2, at most 3, at most 4, at most 5, at most 6, at most 7, at most 8, at most 9, at most 10, at most 11, or at most 12 times in the second cycle. In some embodiments, the method comprises administering the pharmaceutical composition to the subject at most 1 to at most 3, at most 3 to at most 6, at most 6 to at most 9, or at most 9 to at most 12 times in the second cycle. In some embodiments, the method comprises administering the pharmaceutical composition to the subject between 1 to 12 times in the second cycle. In some embodiments, the method comprises administering the pharmaceutical composition to the subject between about 1 to about 3, about 3 to about 6, about 6 to about 9, or about 9 to about 12 times in the second cycle.

In some embodiments, the method comprises administering the pharmaceutical composition to the subject 3 times in the second cycle.

In some embodiments, the method comprises administering the pharmaceutical composition to the subject for three treatment cycles. In some embodiments, the method comprises administering the pharmaceutical composition to the subject at the same dose for each of the three treatment cycles.

In some embodiments, the method comprises administering the pharmaceutical composition to the subject for four treatment cycles. In some embodiments, the method comprises administering the pharmaceutical composition to the subject every 2 weeks in the first cycle and every 4 weeks in the second, third and fourth cycle. In some embodiments, the method comprising administering the pharmaceutical composition at a dose from about 0.0050 to about 0.05 mg/kg or from about 0.0075 to about 0.03 mg/kg. In some embodiments, the method comprises administering the pharmaceutical composition to the subject once a week in the first cycle and once every 4 weeks in the second, third, and fourth cycle.

In some embodiments, the method comprises administering an effective amount of the pharmaceutical composition to the subject.

In some embodiments, the effective amount of the pharmaceutical composition ranges from about 0.001 to about 0.0015, about 0.0015 to about 0.002, about 0.002 to about 0.0025, about 0.0025 to about 0.003, about 0.003 to about 0.0035, about 0.0035 to about 0.004, about 0.004 to about 0.0045, about 0.0045 to about 0.005, about 0.005 to about 0.0055, about 0.0055 to about 0.006, about 0.006 to about 0.0065, about 0.0065 to about 0.007, about 0.007 to about 0.0075, about 0.0075 to about 0.008, about 0.008 to about 0.0085, about 0.0085 to about 0.009, about 0.009 to about 0.0095, or about 0.0095 to about 0.01 mg/kg of the engineered RNA (e.g., recombinant mRNA).

In some embodiments, the effective amount of the pharmaceutical composition ranges from about 0.01 to about 0.015, about 0.015 to about 0.02, about 0.02 to about 0.025, about 0.025 to about 0.03, about 0.03 to about 0.035, about 0.035 to about 0.04, about 0.04 to about 0.045, about 0.045 to about 0.05, about 0.05 to about 0.055, about 0.055 to about 0.06, about 0.06 to about 0,065, about 0.065 to about 0.07, about 0.07 to about 0.075, about 0.075 to about 0.08, about 0.08 to about 0.085, about 0.085 to about 0.09, about 0.09 to about 0.095, or about 0.095 to about 0.1 mg/kg of the engineered RNA.

In some embodiments, the effective amount of the pharmaceutical composition comprises about 0.001, about 0.0015, about 0.002, about 0.0025, about 0.003, about 0.0035, about 0.004, about 0.0045, about 0.005, about 0.0055, about 0.006, about 0.0065, about 0.007, about 0.0075, about 0.008, about 0.0085, about 0.009, about 0.0095, or about 0.01 mg/kg of the engineered RNA.

In some embodiments, the effective amount of the pharmaceutical composition comprises about 0.01, about 0.015, about 0.02, about 0.025, about 0.03, about 0.035, about 0.04, about 0.045, about 0.05, about 0.055, about 0.06, about 0.065, about 0.07, about 0.075, about 0.08, about 0.085, about 0.09, about 0.095, or about 0.1 mg/kg of the engineered RNA.

Provided herein is a method of treating a cancer in a subject in need thereof, the method comprising administering to the subject a pharmaceutical composition comprising an engineered RNA (mRNA), wherein the engineered mRNA comprises a sequence encoding a chimeric fusion protein (CFP), the CFP comprising: (i) an extracellular domain comprising an anti-TROP2 binding domain; and (ii) a CD89 transmembrane domain operatively linked to the extracellular domain; wherein the subject has a cancer selected from the group consisting of cervical cancer, colorectal cancer, esophageal cancer, gastric adenocarcinoma, HR+/HER2− breast cancer, non-small cell lung cancer (NSCLC), ovarian cancer, pancreatic ductal adenocarcinoma, triple negative breast cancer, and urothelial cancer.

In some embodiments, the cancer is advanced or metastatic. In some embodiments, the cancer comprises an advanced or metastatic epithelial cancer.

In some embodiments, the cancer comprises a tumor. In some embodiments, the tumor is associated with cervical cancer, colorectal cancer, esophageal cancer, gastric adenocarcinoma, HR+/HER2− breast cancer, non-small cell lung cancer (NSCLC), ovarian cancer, pancreatic ductal adenocarcinoma, triple negative breast cancer, or urothelial cancer.

In some embodiments, administering the pharmaceutical composition is associated with a reduction of, or amelioration of the tumor in the subject.

In some embodiments, treating comprises reducing the tumor by at least 1%, 2%, 3%, 4%, 5%, 6%, 7%, 8%, 9% or at least 10% after the treatment.

In some embodiments, treating comprises alleviating at least one of the symptoms associated with cancer.

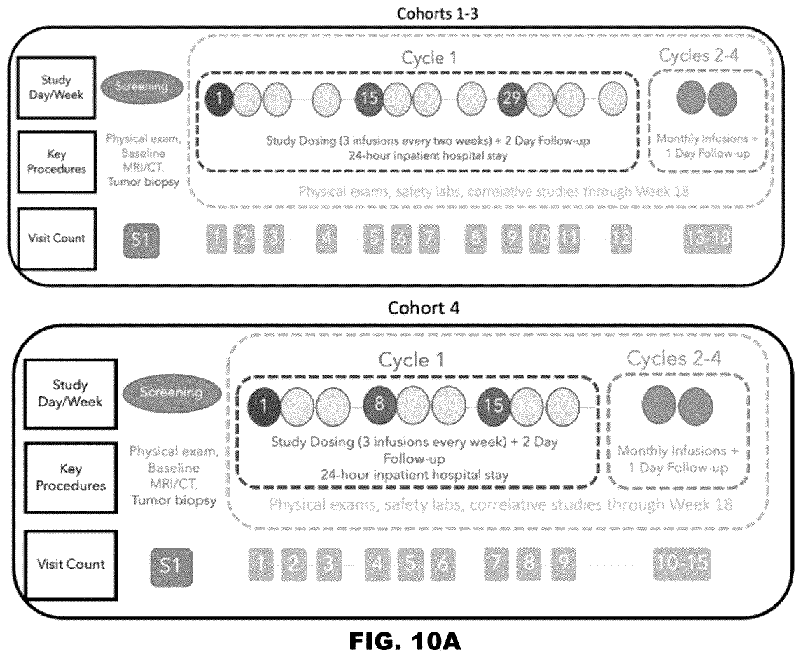

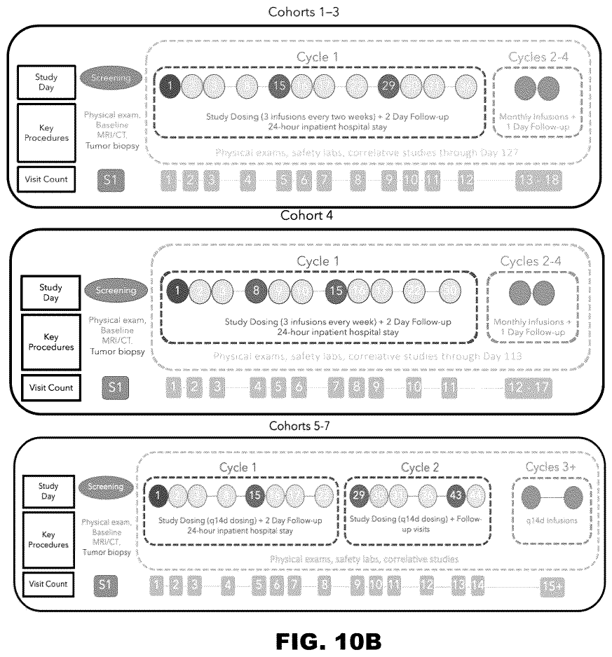

Provided herein is a method of treating a cancer in a subject in need thereof, the method comprising administering to the subject a pharmaceutical composition comprising an engineered RNA, wherein the engineered RNA comprises a sequence encoding a chimeric fusion protein (CFP), the CFP comprising: (i) an extracellular domain comprising an anti-TROP2 binding domain; and (ii) a CD89 transmembrane domain operatively linked to the extracellular domain; wherein the subject does not have: an active CNS metastasis; a carcinomatous meningitis; a clinically significant heart disease; a prior allogenic bone marrow transplantation; a prior solid organ transplant; an active autoimmune disease; an active acute or chronic infection; a liver tumor involvement greater than 50%; or no prior splenectomy. Typically, for first in human studies, study participants (e.g., patients) are selected based on a number of inclusion and exclusion criteria, determined at an initial screening, which include but are not limited to the patient's disease condition and various physiological parameters, described below and more specifically in the examples. Usually, in most circumstances a baseline for any health parameter, e.g. tumor size, stage, patient body weight, body temperature etc., are determined at an initial period prior to onset of the study with the new drug, and further measurements during and after the progression of the therapeutic regimen using the new drug may be compared to this initial measurement, or baseline measurement. However, a baseline for any health/medical parameter at any given period of time during or after the study may be the immediate earlier measurement or any previous measurement that the medical expert in the field may consider suitable and necessary. In some embodiments, a therapeutic regimen or schedule may be described, which may be considered to provide a description of a schedule of administration of a drug at a specific dose or predetermined dose or doses (e.g., amount, concentration), administered at predetermined intervals over a period of time. For example, FIGS. 10A and 10B describe a dosing regimen for the planned study.

Inclusion Criteria: In some embodiments, a subject may be administered a pharmaceutical composition provided herein if she/he/they meet an inclusion criterion. In some embodiments, the inclusion criterion can be: the subject being of an adult age ≥18; and the subject having signed an Informed Consent Form (ICF); for example, additionally the subject having histologically proven, metastatic or advanced epithelial cancer, where in the cancer is selected from the group consisting of: urothelial, cervical, ovarian epithelial, triple-negative breast, HR+/HER2− breast, pancreatic ductal adenocarcinoma, gastric adenocarcinoma, esophageal carcinoma, non-small cell lung, and colorectal; a subject with progressive disease at baseline, refractory or relapsed to standard of care; a subject who has declined standard therapy; a subject with measurable disease based on Response Evaluation Criteria in Solid Tumors (RECIST) criteria v 1.1.; a subject with Eastern Cooperative Oncology Group (ECOG) performance status grade of 0 or 1; a subject with life expectancy of >12 weeks; a subject with echocardiogram (ECHO) or multiple gated acquisition scan showing an ejection fraction ≥50%; a subject with electrocardiogram (ECG) showing no clinically significant abnormality at Screening or showing an average QTc interval <450 msec in males and <470 msec in females (<480 msec for participants with bundle branch block); a subject with oxygen saturation of ≥90% on room air measured by pulse oximetry; a subject with adequate organ function as defined by the following laboratory values at Screening: a. Hemoglobin ≥9.0 g/dL without transfusion support within 2 weeks before Screening; b. Platelet count ≥100,000/μL without transfusion support within 2 weeks before Screening; c. Absolute neutrophil count >1000/mm3 (without granulocyte-colony stimulating factor support within 2 weeks before Screening); d. Creatinine clearance >45 mL/min as calculated using the Cockcroft-Gault equation or creatinine <1.5×ULN; c. ALT<2.5×ULN; ALT<5.0×ULN if known liver disease by cancer; f. AST<2.5×ULN; AST<5.0×ULN if known liver disease by cancer; g. Serum bilirubin <1.5×ULN (≤3.0×ULN if the participant has documented Gilbert syndrome); or h. INR or prothrombin time (PT)≤1.5 ULN unless participant is receiving anticoagulant therapy and INR or PT is within expected or therapeutic range of intended use of anticoagulants; a subject willing and able to provide written informed consent; a subject willing to perform and comply with all study procedures including undergoing study-related biopsies and attending clinic visits as scheduled; a male subject who must abstain from sperm donation during study treatment or for 4 months following last dose of study treatment; a male subject or a female subject who could become pregnant who is willing to practice a highly effective method of contraception.

Lesions situated in a previously irradiated area can be considered measurable if unequivocal progression has been demonstrated in such lesions.

Either Fridericia's or Bazett's formula can be used to correct the QT interval.

Exclusion Criteria: an exclusion criteria may refer to a condition or criterion that when present in a prospective subject for the study will cause elimination of the subject from being entered into the study.

In some embodiments, an exclusion criteria may include for example, a prospective subject having an active CNS metastasis; a carcinomatous meningitis; a clinically significant heart disease; a prior allogenic bone marrow transplantation; a prior solid organ transplant; an active autoimmune disease; an active acute or chronic infection; a liver tumor involvement greater than 50%; or no prior splenectomy.

In some embodiments, a subject is administered a pharmaceutical composition provided herein if they do not meet an exclusion criterion. In some embodiments, the exclusion criterion can be: a subject with known active CNS metastasis and/or carcinomatous meningitis; a subject with previously treated brain metastases provided they are not radiologically stable, (i.e., without evidence of progression for at least 4 weeks by repeat imaging), clinically stable, or without requirement of steroid treatment for at least 14 days prior to the first dose of study intervention; a subject who is pregnant or nursing; a subject >28 days beyond major surgery, including hepatectomy or joint replacement; a subject with prior allogeneic bone marrow transplantation or solid organ transplant; a subject with spinal cord compression not definitively treated with surgery and/or radiation; a subject with uncontrolled pleural effusion, pericardial effusion, or ascites requiring recurrent drainage procedures; a subject with any acute illness including fever (>100.4° F. or >38° C.) within 7 days prior to Day 1; a subject with active systemic bacterial, fungal, or viral infection within 7 days prior to Day 1; a subject with active infection with human immunodeficiency virus (HIV), hepatitis B virus (HBV), hepatitis C virus (HCV), including: a. Positive serology for HIV-1; b. Positive serology for HCV and has not received a documented curative therapy or quantitative RNA PCR is detectable; c. Positive hepatitis B surface antigen or IgM, HBV core antibody unless quantitative DNA PCR is undetectable, and patient is stable on antiviral prophylaxis against HBV reactivation; 10. Other primary malignancies, except: a. Adequately treated basal cell or squamous cell carcinoma b. In situ carcinoma of the cervix or bladder, treated curatively and without evidence of recurrence for at least 2 years prior to the study, or c. A primary malignancy which has been completely resected and in complete remission for at least 2 years; a subject with history of (noninfectious) pneumonitis/interstitial lung disease that required steroids or has current pneumonitis/interstitial lung disease; a subject with prior grade ≥3 immune-related AEs such as pneumonitis, colitis, hepatitis, nephritis; a subject with active autoimmune disease not related to prior therapy for primary malignancy that has required systemic therapy in the last 1 year; a subject with history of symptomatic congestive heart failure (New York Heart Association classes II-IV) or serious active arrhythmias or other clinically significant cardiac disease within 12 months of enrollment; a subject who experienced toxicity from previous anti-cancer therapy defined as toxicities (other than alopecia, or laboratory values listed above) not yet resolved to NCI CTCAE v5.0 Grade≤1 or baseline; a subject who has received: a. Radiotherapy within 2 weeks of first administration of a pharmaceutical composition provided herein; b. Cytotoxic chemotherapy for the treatment within 21 days or 5 half-lives, whichever is shorter of administration of a pharmaceutical composition provided herein; c. Immune therapy for primary malignancy (e.g., monoclonal antibody therapy, checkpoint inhibitors) within 21 days or 5 half-lives, whichever is shorter of first administration of a pharmaceutical composition provided herein; d. Anti-cancer vaccine within 12 weeks of first administration of a pharmaceutical composition provided herein; e. COVID-19 mRNA vaccine within 6 weeks of first administration of a pharmaceutical composition provided herein; a subject who has received a live vaccine ≤6 weeks prior to first administration of a pharmaceutical composition provided herein; a subject who has received packed red blood cells or platelet transfusion within 2 weeks prior to first administration of a pharmaceutical composition provided herein; a subject with history of an allergic reaction to any of the excipients; a subject with enrollment in another interventional clinical trial within 21 days or 5 half-lives of the drug, whichever is shorter of first administration of a pharmaceutical composition provided herein; or a subject with any other condition that, in the opinion of an Investigator, would make the participant unsuitable for the study or unable to comply with the study requirements.

In some embodiments, a subject cannot have tested positive for COVID-19 within 7 days prior to Day 1 and is excluded from the study if tested positive within 7 days prior to Day 1.

In some embodiments, a subject with chronic Grade 2 toxicities (eg, peripheral neuropathy, laboratory values) may be eligible per the discretion of the Investigator and Medical Monitor.

Replacement therapy such as thyroxine, insulin, or physiologic steroid replacement therapy for adrenal or pituitary insufficiency are not considered systemic treatment.

A baseline may be considered as the most recent assessment performed prior to first dose of study treatment. Baseline assessments may be performed within the period defined in the protocol eligibility criteria.

Measurable lesions: Except for lymph nodes as described below, measurable lesions are defined as those that can be accurately measured in at least 1 dimension (longest diameter to be recorded) as ≥10 mm with CT scan (if CT scans have slice thickness greater than 5 mm the minimum size for a measurable lesion is twice the slice thickness).

To be considered pathologically enlarged and measurable, a lymph node must be ≥15 mm in short axis when assessed by CT scan (CT scan slice thickness recommended to be no greater than 5 mm). At baseline and in follow-up, only the short axis will be measured and recorded.

MRI may be substituted for contrast-enhanced CT for lesions at some anatomical sites, but not for lesions in the lungs. The minimum size for measurability is the same as for CT (10 mm) as long as the scans are performed with slice thickness of 5 mm and no gap. If MRI is performed with thicker slices, the size of a measurable lesion at baseline should be twice the slice thickness. In the event there are interslice gaps, this also needs to be considered in determining the size of measurable lesions at baseline.

Nonmeasurable lesions: In some embodiments, all other lesions (or sites of disease) excepting the measurable lesions discussed above, including small lesions (longest diameter <10 mm or pathological lymph nodes with ≥10 to <15 mm short axis), may be considered nonmeasurable. Lymph nodes that have a short axis <10 mm may be considered nonpathological and may not be recorded or followed. Bone lesions, leptomeningeal disease, ascites, pleural/pericardial effusions, lymphangitis cutis/pulmonitis, and abdominal masses (not followed by CT or MRI) may be considered as nonmeasurable. Following a time point response of CR, non-target lymph node lesions and new lymph node lesions may be measured to determine if they are or become pathologic in size.

Target lesions: In some embodiments, target lesions are measurable lesions up to a maximum of 2 lesions per organ and 5 lesions in total, representative of all involved organs, may be identified as target lesions and measured and recorded at baseline. Target lesions may be selected based on their size (lesions with the longest diameter), be representative of all involved organs, and be those that lend themselves to reproducible repeated measurements. It may be the case that, on occasion, the largest lesion does not lend itself to reproducible measurement in which circumstance the next largest lesion that can be measured reproducibly should be selected. Target lesions may be measured at each assessment (longest axis for nonnodal lesions, shortest axis for measurable malignant nodal lesions).

Nontarget lesions: In some embodiments, other lesions (or sites of disease) than those discussed under target lesions above including all non-measurable lesions (including pathological lymph nodes with ≥10 to <15 mm short axis) and all measurable lesions over and above the 5 target lesions may be identified as non-target lesions and recorded at baseline. Measurements of these lesions are generally not required, but the presence, absence, or in rare cases unequivocal progression of each is to be recorded throughout follow-up. Lymph nodes that have a short axis <10 mm may be considered non-pathological and may not to be recorded or followed. Following a time point response of CR, non-target lymph node lesions and new lymph node lesions may be measured to determine if they are or become pathologic in size.

In some embodiments, in order to be considered progression of non-target lesions in the presence of measurable disease, unequivocal progression may be defined as substantial worsening in non-target disease such that, even in the presence of SD or PR in target disease, the overall tumor burden has increased sufficiently to merit discontinuation of the therapy. Cystic lesions other than metastatic cystic lesions may not be considered for measurement. Lytic bone lesions or mixed lytic-blastic lesions, with identifiable soft tissue components, that can be evaluated by cross-sectional imaging techniques such as CT or MRI can be considered as measurable lesions if the soft tissue component meets the definition of measurability described above. Blastic bone lesions are non-measurable. Chest x-ray may not be used for response assessment in this study. CT scans have slice thickness greater than 5 mm, the minimum size for a measurable lesion is twice the slice thickness. MRI is also acceptable in certain situations (eg, for body scan) except for lung. Bone scans and PET scans may be used for bone lesion surveillance but may not be employed for RECIST evaluations. CT or MRI scan may be used to confirm results of bone scans for this purpose. Preferred method for confirmation is MRI. Tumor markers may not be used to determine PD in this study.

Ascites and effusions: New or worsening ascites or effusions are to be considered malignant unless deemed unlikely to be of non-neoplastic origin by cytology or histology.

### Time Point Assessments

The frequency and schedule of tumor assessments is defined in the protocol. The schedule is to be maintained regardless of whether study treatment is reduced, withheld, delayed, or discontinued.

At baseline, tumors and lymph nodes may be classified and documented as target or nontarget lesions as described above. It is possible to record multiple nontarget lesions involving the same organ as a single item (eg, ‘multiple liver metastases’). At all postbaseline (follow-up) evaluations the baseline classification (target, nontarget) may be maintained and lesions may be documented and described in a consistent fashion over time (eg, recorded in the same order on source documents).

At each assessment, a sum of the diameters (longest for nonnodal lesions, short axis for nodal lesions) for all target lesions can be calculated and included in source documents. The baseline sum of the diameters (SoD) may be used as reference to further characterize any objective tumor regression in the measurable dimension of the disease. The lowest SoD (nadir) since (and including) the baseline value may be used as reference for evaluating progression.

After baseline, target lesions may have the actual size documented, if possible, even if the lesions become very small. If in the opinion of the radiologist the lesion has likely disappeared, 0 mm may be recorded. If the lesion is present but too small to measure, an indicator for ‘too small to measure’ may be included in source documents.

In some embodiments, for target lesions, measurements may be taken and recorded in metric notation. Nontarget lesions may be assessed qualitatively (present, resolved, or unequivocal progression) and new lesions, if any, may be documented separately. At each evaluation, progression status is to be determined based upon the time point status for target lesions, nontarget lesions, and new lesions. Finding of new lesions should not be attributable to differences in scanning technique, change in imaging modality or findings thought to represent something other than tumor. Necrosis of pre-existing lesions as part of a response to treatment should be excluded before defining a ‘new’ cystic lesion. A lesion identified on a follow-up study in an anatomical location that was not scanned at baseline is considered a new lesion. If a new lesion is equivocal because of its small size, repeat scans need to confirm there is definitely a new lesion, and progression should be declared using the date of the initial scan.

In some embodiments, a time point progression may not be based solely on bone scan findings. Bone scans are to be used to direct corroborative imaging with CT/MRI if necessary. CT/MRI findings may be used for the determination of progression.

### EXAMPLES

### Example 1. First-In-Human Phase I Study in Human

This example describes a Phase I, open-label, first-in-human, multiple ascending dose to investigate the safety, pharmacokinetics, pharmacodynamics and preliminary efficacy of TROP2 expressing second generation constructs in adults with TROP2+ metastatic colorectal cancer. In this study, the constructs are programmed to be administered anti-TROP2-Fc-alpha fusion receptor in form of an mRNA encoding the construct encapsulated in lipid nanoparticle.

Number of Subjects Planned: Up to 20 safety and efficacy evaluable subjects.

Adults 18 years of age inclusive or older will be screened. Subjects who provide written informed consent and meet all inclusion and exclusion criteria will be entered into the trial. The study will be divided into two parts. Part A will be a multiple ascending dose to determine safety, tolerability, and pharmacokinetics (PK) of anti-TROP2 second generation chimeric fusion protein (anti-TROP2-CD89 CAR) in subjects with TROP2+ LM; Part B will be a dose expansion to determine further safety, tolerability, and PK, as well as preliminary efficacy in patients with TROP2+ CRLM.

The subjects will be administered the formulation of an effective amount of mRNA encoding the anti-TROP2 second generation chimeric fusion protein in a lipid nanoparticle intravenously over 60 minutes. The starting dose and dose regimen will be determined after completion of PK and safety studies in non-human primates.

Baseline evaluations within 4 weeks of the scheduled start of treatment include patient history, physical examination with vital signs and performance status, CT or MRI scans, CBC with differential and platelet count, routine serum chemistries, urinalysis, INR/PTT, EKG, and serum samples for human anti-human antibody (HAHA). In women of childbearing potential, a urine or serum b-HCG is also required within one week of treatment. TROP2 expression will be confirmed by immunohistochemistry at a central laboratory on an archived biopsy specimen obtained within the last 6 months or on a fresh biopsy specimen during screening.

All subjects will receive weekly infusions of anti-TROP2 second generation chimeric fusion protein for the first 12 weeks. Dosing can be continued in the absence of progression of disease or unacceptable toxicity through Week 48. After Week 12 patients may continue in the study through Week 48, but the dose regimen will be modified to once every two weeks. If a patient progresses while receiving once every two weeks dosing, they may escalate to weekly dosing. If a complete responder discontinues therapy at any time on study and develops progressive disease, they may resume dosing on the dose and dose schedule when they discontinued. If a patient has a partial response (PR) and surgical resection of the CRLM is possible, patient may discontinue treatment and opt for tumor resection. Patients who undergo surgical resection will be followed through Week 48. If patient develops progressive disease, they may resume dosing on the dose and dose schedule when they discontinued.

All patients should be closely monitored over the course of their treatment. NCI CTCAE version 5.0 will be used to grade all adverse event and to provide dose reduction, delay, or cessation guidelines in the event of treatment-related toxicity. All patients will also have CT/MRI scans throughout the study to assess for progression of disease as well as response to study drug

The development of any Grade 2 or 3 treatment-related toxicity at the time of a scheduled treatment day will delay treatment by one week. If toxicity has resolved to Grade 1 or lower at that time, treatment may continue with the dose reduced by 25%. If the toxicity reoccurs, the treatment can continue with a 50% reduction from initial dose, and if the toxicity worsened treatment will be permanently discontinued. The decision to continue or discontinue treatment is based solely on physician discretion. The development of any Grade 4 treatment related toxicities, the patient should be discontinued permanently.

In Part A of the study a Continuous Reassessment Method (CRM) will be used to determine the recommended dose for Part B. Primary assessment of safety and tolerability will be at Day 28. Primary assessment of efficacy will be at 12 weeks. Subjects may enroll in a long-term extension study at 48 weeks if they have stable disease (SD), partial response (PR), or complete response (CR).

In Part B the maximum acceptable dose (MAD) from Part A will be assessed for safety, tolerability, and efficacy in patients with CRLM. Primary assessment of efficacy will be objective response rate (ORR) at 12 weeks.

In the event of treatment termination due to unacceptable toxicity the patient will continue in the study until the occurrence of progression of disease, at which time an end-of-study evaluation will be performed, with additional follow-up required until resolution or stabilization of any treatment-related toxicity. All patients will be followed for survival.

To ensure the safety of study subjects, the first two subjects in each dose cohort will be staggered by an interval of 14 days. If there are no safety concerns, then the remaining subjects may enroll at that dose level simultaneously.

The pharmaceutical composition comprises an scFv comprising a heavy chain and a light chain, the heavy chain comprising a CDR3 having a sequence GGFGSSYWYFDV, and the light chain comprising a CDR3 having a sequence QQHYITPLT. In some embodiments, the heavy chain further comprises CDR1 sequence of NYGMN, and a CDR2 sequence of WINTYTGEPTYTDDFKG; and the light chain further comprises a CDR1 sequence of KASQDVSIAVA, and a CDR2 sequence of SASYRYT. The pharmaceutical composition described above comprises a CD89 transmembrane domain having a sequence LIRMAVAGLVLVALLAILV.

An exemplary anti-TROP2 second generation chimeric fusion protein (e.g., a drug product) comprising an anti-TROP2 scFv comprising a heavy chain variable domain having a sequence (CDRs highlighted according to Kabat naming convention)

QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYFDVWGQGSLVTVSSG.

An exemplary anti-TROP2 chimeric fusion protein comprising an antiTROP2 scFv comprises a light chain variable domain having sequence

DIQLTQSPSSLSASVGDRVSITCKASQDVSIAVAWYQQKPGKAPKLLIY
SASYRYTGVPDRFSGSGSGTDFTLTISSLQPEDFAVYYCQQHYITPLTF
GAGTKVEIKR

An exemplary TROP2 scFv construct has the following sequence,

MWLQSLLLLGTVACSISQVQLQQSGSELKKPGASVKVSCKASGYTFTNY
GMNWVKQAPGQGLKWMGWINTYTGEPTYTDDFKGRFAFSLDTSVSTAYL
QISSLKADDTAVYFCARGGFGSSYWYFDVWGQGSLVTVSSGGGGSGGGG
SGGGGSDIQLTQSPSSLSASVGDRVSITCKASQDVSIAVAWYQQKPGKA
PKLLIYSASYRYTGVPDRFSGSGSGTDFTLTISSLQPEDFAVYYCQQHY
ITPLTFGAGTKVEIKRGSGGSDSIHQDYTTQNLIRMAVAGLVLVALLAI
LVENWHSHTALNKEASADVAEPSWSQQMCQPGLTFARTPSVCK

The bold letters represent the signal peptide sequence. A mature protein may lack this sequence, and one of skill in the art can interpret the protein sequence as expressed without the signal peptide sequence. The underlined regions are the CDR sequence of the scFV that binds to TROP2 in sequence, CDR1, CDR2, CDR3 for the heavy and light chains. The italicize alphabets represent the amino acid sequence of the CD89 TMD.

An exemplary TROP2 scFv product used for in vivo study can comprise a sequence

QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYEDVWGQGSLVTVSSGGGGGGGGSGGGGSDIQLTQSPSSLS
ASVGDRVSITCKASQDVSIAVAWYQQKPGKAPKLLIYSASYRYTGVPDR
FSGSGSGTDFTLTISSLQPEDFAVYYCQQHYITPLTFGAGTKVEIKRSG
GGGAAADYKDDDDKGSDSIHQDYTTQNLIRMAVAGLVLVALLAILVENW
HSHTALNKEASADVAEPSWSQQMCQPGLTFARTPSVCK

An exemplary TROP2 scFv product used for in vivo study can comprise a sequence

QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYFDVWGQGSLVTVSSGGGGSGGGGSGGGGSDIQLTQSPSSL
SASVGDRVSITCKASQDVSIAVAWYQQKPGKAPKLLIYSASYRYTGVPD
RFSGSGSGTDFTLTISSLQPEDFAVYYCQQHYITPLTFGAGTKVEIKRG
SGGSDSIHQDYTTQNLIRMAVAGLVLVALLAILVENWHSHTALNKEASA
DVAEPSWSQQMCQPGLTFARTPSVCK

This approach takes advantage of the natural propensity for intravenous (i.v) delivery.

The proprietary mRNA construct encodes for a receptor consisting of a TROP2-targeted scFv (derived from the complementarity-determining regions (CDR) of sacituzumab), as well as the transmembrane domain and cytoplasmic tail of CD89. Upon administration of the anti-TROP2 binder, the LNP is taken up by numerous cell types, however, a functional CAR can only be expressed on the surface of cells that also express the Fc receptor common gamma chain, predominately myeloid cells. The common gamma chain includes the immunoreceptor tyrosine-based activation motif (ITAM) domains necessary for cellular signaling. Upon scFV recognition of TROP2, the intracellular signaling domains become activated, resulting in tumor cell phagocytosis, inflammatory cytokine production and presentation of tumor antigens to T cells.

The primary objective of this study is to evaluate the safety and tolerability of anti-TROP2 second generation chimeric fusion protein in subjects with TROP2⁺ metastatic colorectal cancer, and to establish the maximum acceptable dose (MAD) and recommended Phase 2 dose (RP2D), based on observed adverse events (AEs) including all potential dose limiting toxicities (DLTs).

The secondary objectives of the this study are to determine: (i) Pharmacokinetics (ii) Objective response of anti-TROP2 second generation chimeric fusion protein [Time Frame: Up to 12 months] (iii) Duration of response.

Secondary objectives further include:
Area under the curve [Time frame: Up to 12 months]
Maximum plasma concentration [Time Frame: Up to 12 months]
Time of maximum plasma concentration [Time Frame: Up to 12 months]
Half-life [Time Frame: Up to 12 months]
Objective response of anti-TROP2 second generation chimeric fusion PROTEIN [Time Frame: Up to 12 months]
Duration of response

The exploratory objectives of this study are to determine (a) Preliminary efficacy including Progression Free Survival (PFS) and Overall Survival (OS) (b) Conversion to surgical candidate (c) Correlative biomarker studies.

In general, the exploratory objective include:
Progression Free Survival (PFS)
Overall Survival (OS)
Conversion to surgical candidate
Development of anti-drug antibodies (ADA) to anti-TROP2 second generation chimeric fusion protein
The level of TROP2 expression in tumors to identify potential biomarkers of response
The treatment-related effects of cytokine and chemokine production, TCR expansion and cell phenotype in the blood to identify potential biomarkers of response.
Assess the treatment-related effects to tumor architecture and cell phenotype to identify potential biomarkers of response

The overall study design includes a multicenter, open-label, Phase 1 first-in-human study with dose cohort expansion to assess the safety, tolerability, pharmacokinetics (PK), and efficacy of the anti-TROP2 second generation binder in subjects with TROP2+ colorectal liver metastases. Adults 18 years of age inclusive or older will be screened. Subjects who provide written informed consent and meet all inclusion and exclusion criteria will be entered into the trial. The study will be divided into two parts. Part A will be a multiple ascending dose to determine safety, tolerability, and PK of anti-trop2 second generation chimeric fusion protein in subjects with TROP2+ liver metastasis (LM); Part B will be a dose expansion to determine further safety, tolerability, and PK, as well as preliminary efficacy in patients with TROP2+ colorectal liver metastasis (CRLM). The main eligibility criteria are delineated below:

Inclusion Criteria: Subjects are eligible for the study if the following criteria are met:

1. Ascending dose portion of the trial (Part A): Histologically proven, metastatic TROP2+ LM with progressive disease at baseline, refractory to standard of care or who have declined standard therapy.

2. Cohort extension portion of the trial (Part B): Histologically proven, metastatic TROP2+ CRLM with progressive disease at baseline, refractory to standard of care or who have declined standard therapy.

3. Tumor TROP2+ with a score of 2+ or 3+ as determined by immunohistochemistry (IHC). This will be performed at a central laboratory.

4. Measurable disease based on RECIST criteria v 1.1.

Exclusion Criteria: Subjects are excluded from the study if any of the following criteria are met:

1. Known active CNS metastasis and/or carcinomatous meningitis.

2. Prior allogenic bone marrow transplantation or solid organ transplant.

3. Active autoimmune disease

4. Active acute or chronic infections

5. Liver tumor involvement greater than 50%.

6. No prior splenectomy. Patients with a prior partial splenic artery embolization may be eligible per the discretion of the Investigator.

Duration of Treatment: There will be a total of 3 cycles. Each cycle is weekly dosing for 4 weeks. After 3 cycles, in the absence of disease progression, patients may continue in the study through week 48 at the Investigator's discretion, but the dose regimen will be modified to once every two weeks. If patients develop progressive disease, patients can escalate to weekly dosing. If patient progresses on weekly dosing, treatment will be discontinued.

### Dose Limiting Toxicity Definition:

A DLT is defined using the Common Terminology Criteria for Adverse Events (CTCAE) v5.0 as specified. All toxicities will be considered at least “possibly” related to ANTI-TROP2 SECOND GENERATION CHIMERIC FUSION PROTEIN unless they have no temporal association with the administration of ANTI-TROP2 SECOND GENERATION CHIMERIC FUSION PROTEIN but rather is related to other etiologies such as concomitant medications or conditions, or subject's underlying disease. For this study, the following are considered DLTs if they occur within 28 days of the first dose (Day 28), except as noted below:
Death
Any CTCAE Grade 4 toxicity
CTCAE Grade 3 toxicity in vital organs (central nervous system [CNS], heart and lung)
CTCAE Grade 3 toxicity that does not decrease to £ Grade 2 within 72 hours with maximal supportive care (eg, for nausea, vomiting, diarrhea), except renal and hepatic laboratory abnormalities.
CTCAE Grade 3 toxicity that does not decrease to £ Grade 2 within 7 days for renal and hepatic laboratory abnormalities
Any Grade 3 infusion related reaction lasting >24 hours and patient was premedicated with H1 antagonist, H2 antagonist, and corticosteroids
Exceptions to DLT Grade 3 or 4 criteria include:
   Laboratory values that are not considered clinically significant by the investigator

### Study Halting Rules:

At any dose level, should a subject experience a DLT as listed above, further dosing of the subject and enrollment will be temporarily halted, and the steering committee will be immediately notified and convened to review the safety data. Following review of the safety data, the steering committee will recommend, based on frequency of DLTs, whether to:
Continue the study as planned, including administration of the additional doses
Expand the dose cohort to obtain additional safety information
Decrease the dose either to a previous lower dose or to an intermediate dose
Eliminate administration of additional doses
Terminate the study and follow all subjects for safety

### Statistical Considerations:

Dose progression will be determined by a Continuous Replacement Model

### Endpoints/Criteria for Evaluation:

### Safety:

AEs, IRs, serious AEs (SAEs). Clinically significant abnormal physical exam findings and laboratory results will be reported as AEs.
Vital signs
Serum chemistries, hematology, T cell count, and plasma cytokines

Anti-TROP2 binder Cell Kinetics: pharmacokinetic parameters (e.g., Cmax, Tmax, T½) of the drug/product in blood after each infusion by qPCR

ADA: Proportion of subjects who develop ADA as measured by ELISA

### Clinical Response:

RECIST v1.1
Conversion to surgical candidate

### Correlative Markers of Response:

Tumor penetration of cells by IHC Image Mass Cytometry
Recruitment and trafficking of other immune cells by mass cytometry imaging
Upregulation of cytokine and chemokine production by transcriptional analysis

TABLE 6
The schedule of events
Schedule of Events

| | Screening | | Cycle 1 | | | | | | | |
|---|---|---|---|---|---|---|---|---|---|---|
| | & | | Dose | | | Dose | | | Dose | |
| | Prep | | 1 | | | 2 | | | 3 | |
| | Study Day | | | | | | | | | |
| | S1 | Baseline | D 1 | D 2 | D 3 | D 8 | D 9 | D 10 | D 15 | D 16 |
| | Study Week/Month | | | | | | | | | |
| | | | W 1 | | | W 2 | | | W 3 | |
| | Window | | | | | | | | | |
| | −28 | −5 | | 0 | 0 | 1 | 0 | 0 | 1 | 0 |
| Informed Consent | X | | | | | | | | | |
| Confirm eligibility | X | | | | | | | | | |
| Medical History | X | | | | | | | | | |
| Complete Physical | X | | | | | | | | | |
| Exam | | | | | | | | | | |
| Targeted Physical | | | X | X | X | X | X | X | X | X |
| Exam | | | | | | | | | | |
| Vital Signs | X | | X | X | X | X | X | X | X | X |
| ECOG Performance | X | | | | | | | | | |
| Score | | | | | | | | | | |
| 12 Lead ECG | X | | | | | | | | | |
| ECHO | | | | | | | | | | |
| MRI/CT | X | | | | | | | | | |
| Pulse Oximetry | X | | X | | | X | | | X | |
| Laboratory: | | | | | | | | | | |
| Serum Chemistry | X | X | X | | | X | | | X | |
| CEA | X | | X | | | X | | | X | |
| CBC w/Differential, | X | X | X | X | X | X | X | X | X | X |
| Platelets | | | | | | | | | | |
| Uninalysis | X | | X | | | | | | | |
| INR and aPTT | X | | X | | | X | | | X | |

| | | | | | | | Cycles 4- | |
|---|---|---|---|---|---|---|---|---|
| | | | | | Cycle | | 12 | |
| | Cycle 1 | | | | 2, 3 | | Dose | End |
| | | Dose | | | Dose | | 13- | of |
| | | 4 | | | 5-12 | Biopsy | 30 | Study |
| | Study Day | | | | | | | |
| | D 17 | D 22 | D 23 | D 24 | D 29-D 90 | D 91 | D 91-D 248 | |
| | Study Week/Month | | | | | | | |
| | | | | | W 5- | | W 13- | |
| | | W 4 | | | W 12 | W 12 | W 48 | |
| | Window | | | | | | | |
| | | | | | | | +/−7 | |
| | 0 | 1 | 0 | 0 | ±3 | ±3 | days | |
| Informed Consent | | | | | | | | |
| Confirm eligibility | | | | | | | | |
| Medical History | | | | | | | | |
| Complete Physical | | | | | | | | |
| Exam | | | | | | | | |
| Targeted Physical | X | X | X | X | X | | X | X |
| Exam | | | | | | | | |
| Vital Signs | X | X | X | X | X | | X | X |
| ECOG Performance | | | | | | | | |
| Score | | | | | | | | |
| 12 Lead ECG | | | | | | | | |
| ECHO | | | | | | | | |
| MRI/CT | | X | | | X | | X | X |
| Pulse Oximetry | | | | | | | | |
| Laboratory: | | | | | | | | |
| Serum Chemistry | | X | | | X | | X | X |
| CEA | | X | | | X | | X | X |
| CBC w/Differential, | X | X | X | X | X | | X | X |
| Platelets | | | | | | | | |
| Uninalysis | | | | | | | | X |
| INR and aPTT | | X | | | X | | X | X |

### Example 2. A Multicenter, Open-Label, Phase 1 First-In-Human Study to Assess the Safety, Tolerability in Advanced Epithelial Cancer

The pharmaceutical composition comprises an scFv comprising a heavy chain and a light chain, the heavy chain comprising a CDR3 having a sequence GGFGSSYWYFDV, and the light chain comprising a CDR3 having a sequence QQHYITPLT. In some embodiments, the heavy chain further comprises a CDR1 sequence of NYGMN, and a CDR2 sequence of WINTYTGEPTYTDDFKG; and the light chain further comprises a CDR1 sequence of KASQDVSIAVA, and a CDR2 sequence of SASYRYT. The pharmaceutical composition described above comprises a CD89 transmembrane domain having a sequence LIRMAVAGLVLVALLAILV.

The study has 4 Cohorts. Each Cohort has 4 Cycles. For Cohorts 1-3, the dosing regimen will be every 14 days for 3 doses, followed by administration once every 28 days for three doses. For Cohort 4, the dosing regimen will be modified. Participants will receive one dose of anti-TROP2-chimeric antigen receptor investigational drug every week for 3 doses, followed by administration once every 28 days for three additional doses.

The conditions treated in this clinical study include malignant epithelial tumors.

The study type is interventional, Phase 1.

The study model is a Single Group Assignment. Number of arm, 1.

The study anticipates to enroll 48 patients.

### Primary Outcome Measure:

1. To evaluate the safety and tolerability of the anti-TROP2-chimeric antigen receptor investigational drug through incidence of Adverse Events. Adverse Events will be graded according to the NCI-CTCAE, version 5.0 [Time Frame: Up to Week 20].

2. To establish the maximum tolerated dose (MTD) based on dose limiting toxicities (DLTs) [Time Frame: Up to Week 20]

3. to establish the maximum tolerated dose (MTD) based on recommended Phase 2 dose (RP2D) [Time Frame: Up to Week 20]

### Secondary Outcome Measure:

4. To further characterize the safety of the anti-trop2-chimeric antigen receptor investigational drug through incidence of Adverse Events. Adverse Events will be graded according to the NCI-CTCAE, version 5.0 [Time Frame: Up to Week 20].

5. To determine the pharmacokinetics (PK) of the anti-trop2-chimeric antigen receptor investigational drug PK Parameter: Plasma concentrations [Time Frame: Up to Week 20]

6. To determine the pharmacokinetics (PK) of the anti-trop2-chimeric antigen receptor investigational drug PK Parameter: Area under the curve [Time Frame: Up to Week 20]

7. To determine the pharmacokinetics (PK) of the anti-trop2-chimeric antigen receptor investigational drug PK Parameter: Maximum observed plasma concentration [Time Frame: Up to Week 20]

8. To determine the pharmacokinetics (PK) of the anti-trop2-chimeric antigen receptor investigational drug PK Parameter: Time of maximum observed plasma concentration [Time Frame: Up to Week 20]

9. To determine the pharmacokinetics (PK) of the anti-trop2-chimeric antigen receptor investigational drug PK Parameter: Apparent terminal Half-life [Time Frame: Up to Week 20]

10. To determine the pharmacokinetics (PK) of the anti-trop2-chimeric antigen receptor investigational drug PK Parameter: Plasma Clearance [Time Frame: Up to Week 20]

11. To determine the pharmacokinetics (PK) of the anti-trop2-chimeric antigen receptor investigational drug PK Parameter: Volume of Distribution [Time Frame: Up to Week 20]

12. To determine the pharmacokinetics (PK) of the anti-trop2-chimeric antigen receptor investigational drug PK Parameter: Mean residence time [Time Frame: Up to Week 20]

13. To determine the pharmacokinetics (PK) of the anti-trop2-chimeric antigen receptor investigational drug PK Parameter: terminal rate constant [Time Frame: Up to Week 20]

14. Determine rate of ICANS—For grading of potential immune effector cell-associated neurotoxicity syndrome (ICANS), use of the 10-point immune effector cell-associated encephalopathy (ICE) screening tool [Time Frame: Up to Week 20]

15. Determine rate of Grade 3-5 CRS [Time Frame: Up to Week 20]

Eligibility: The following criteria are followed for enrolling patients

Minimum Age: 18 Years; Sex: All,

The study does not accept healthy volunteer.

### Criteria: Inclusion Criteria;

1. Adults age ≥18 inclusive at the time the Informed Consent Form (ICF) is signed.

2. Histologically proven, metastatic or advanced epithelial cancer including the following cancer types:
a. Urothelial
b. Cervical
c. Ovarian epithelial
d. Triple-negative breast
e. HR+/HER2− breast
f. Pancreatic ductal adenocarcinoma
g. Gastric adenocarcinoma
h. Esophageal carcinoma
i. Non-small cell lung
j. Colorectal

3. Progressive disease at baseline, refractory or relapsed to standard of care or who have declined standard therapy.

4. Measurable disease based on Response Evaluation Criteria in Solid Tumors (RECIST) criteria v 1.1.

5. Eastern Cooperative Oncology Group (ECOG) performance status grade of 0 or 1.

6. Life expectancy of >12 weeks.

7. Echocardiogram (ECHO) or multiple gated acquisition scan showing an ejection fraction greater than or equal to 50%.

8. Electrocardiogram (ECG) showing no clinically significant abnormality at Screening or showing an average QTc interval <450 msec in males and <470 msec in females (<480 msec for participants with bundle branch block). Either Fridericia's or Bazett's formula may be used to correct the QT interval.

9. Oxygen saturation of greater than or equal to 90% on room air measured by pulse oximetry.

10. Adequate organ function as defined by laboratory values at Screening.

11. Willing and able to provide written informed consent.

12. Willing to perform and comply with all study procedures including undergoing study-related biopsies and attending clinic visits as scheduled.

13. Men must abstain from sperm donation during study treatment or for 4 months following last dose of study treatment.

14. Men and WOCBP must be willing to practice a highly effective method of contraception.

### Exclusion Criteria:

1, Known active CNS metastasis and/or carcinomatous meningitis. Participants with previously treated brain metastases may participate provided they are radiologically stable, (ie, without evidence of progression for at least 4 weeks by repeat imaging), clinically stable, and without requirement of steroid treatment for at least 14 days prior to the first dose of study intervention.

2. Pregnant or nursing women.

3. Must be >28 days beyond major surgery, including hepatectomy or joint replacement.

4. Prior allogeneic bone marrow transplantation or solid organ transplant.

5. Spinal cord compression not definitively treated with surgery and/or radiation.

6. Uncontrolled pleural effusion, pericardial effusion, or ascites requiring recurrent drainage procedures.

7. Any acute illness including fever (>100.4° F. or >38° C.) within 7 days prior to Day 1.

8. Active systemic bacterial, fungal, or viral infection within 7 days prior to Day 1. Participant cannot have tested positive for COVID-19 within 7 days prior to Day 1.

9. Active infection with human immunodeficiency virus (HIV), hepatitis B virus (HBV), hepatitis C virus (HCV).

10. Other primary malignancies, except: a. Adequately treated basal cell or squamous cell carcinoma b. In situ carcinoma of the cervix or bladder, treated curatively and without evidence of recurrence for at least 2 years prior to the study, or c. A primary malignancy which has been completely resected and in complete remission for at least 2 years.

11. History of (noninfectious) pneumonitis/interstitial lung disease that required steroids or has current pneumonitis/interstitial lung disease.

12. Prior grade ≥3 immune-related AEs such as pneumonitis, colitis, hepatitis, nephritis; prior dermatitis and endocrinopathies are allowed provided corticosteroids are no longer required and endocrine-replacement therapy is stable and discontinued from prior therapy.

13. Active autoimmune disease not related to prior therapy for primary malignancy that has required systemic therapy in the last 1 year.

14. History of symptomatic congestive heart failure (New York Heart Association classes II-IV) or serious active arrhythmias or other clinically significant cardiac disease within 12 months of enrollment.

15. Toxicity from previous anti-cancer therapy defined as toxicities (other than alopecia, or laboratory values listed above) not yet resolved to NCI CTCAE v5.0 Grade≤1 or baseline. Participants with chronic Grade 2 toxicities (eg, peripheral neuropathy, laboratory values) may be eligible per the discretion of the Investigator and Medical Monitor.

16. Has received:
a. Radiotherapy within 2 weeks of first administration of the anti-trop2-chimeric antigen receptor investigational drug.
b. Cytotoxic chemotherapy for the treatment within 21 days or 5 half-lives, whichever is shorter, of administration of the anti-TROP2-chimeric antigen receptor investigational drug.
c. Immune therapy for primary malignancy (eg, monoclonal antibody therapy, checkpoint inhibitors) within 21 days or 5 half-lives, whichever is shorter of first administration of the anti-trop2-chimeric antigen receptor investigational drug.
d. Anti-cancer vaccine within 12 weeks of first administration of the anti-trop2-chimeric antigen receptor investigational drug e. COVID-19 mRNA vaccine within 6 weeks of first administration of the anti-trop2-chimeric antigen receptor investigational drug.

17. Has received a live vaccine≤6 weeks prior to first administration of the anti-trop2-chimeric antigen receptor investigational drug.

18. Has received packed red blood cells or platelet transfusion within 2 weeks prior to first administration of the anti-trop2-chimeric antigen receptor investigational drug.

19. History of an allergic reaction to any of the excipients.

20. Enrollment in another interventional clinical trial within 21 days or 5 half-lives of the drug, whichever is shorter, of first administration of the anti-trop2-chimeric antigen receptor investigational drug.

21. Any other condition that, in the opinion of the Investigator, would make the participant unsuitable for the study or unable to comply with the study requirements.

### Example 3. Non-Clinical Studies: Data from Mouse Gp75+ Tumor Model

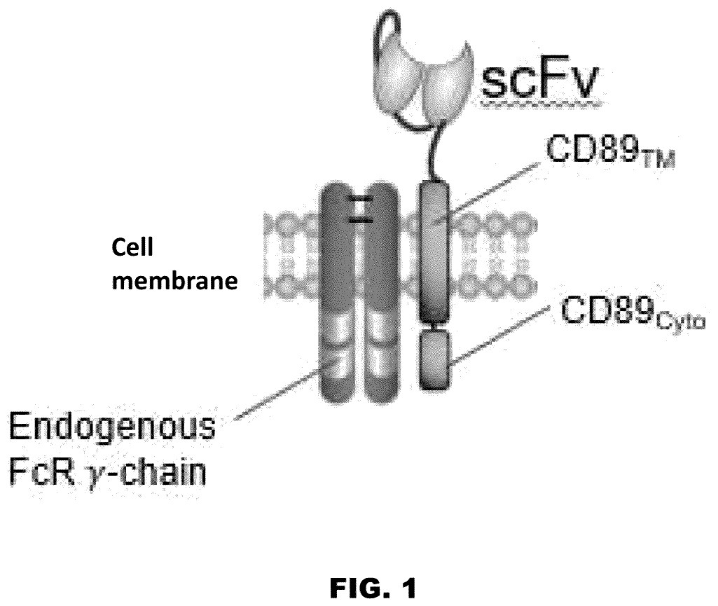

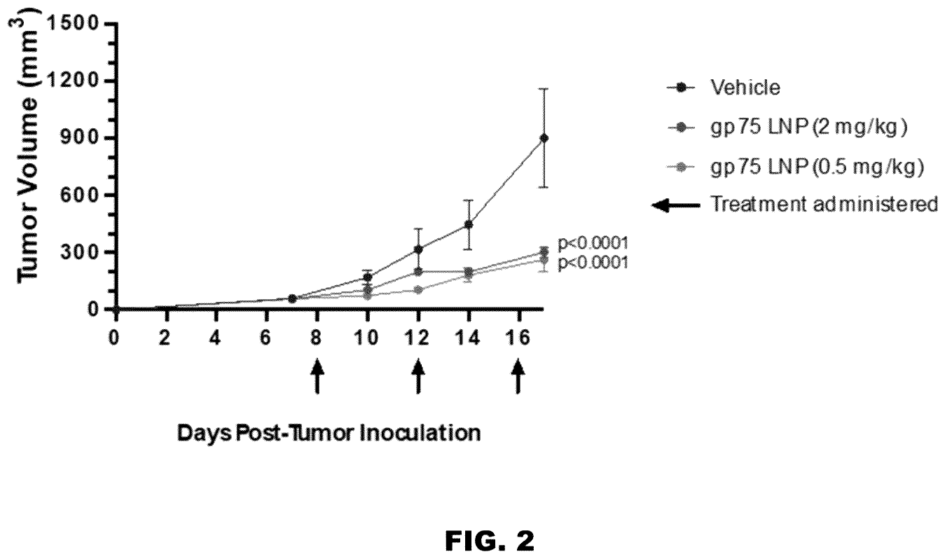

The objective of these studies was to evaluate the anti-tumor efficacy of myeloid cells that were programmed with anti-GP75-CD89 LNP. The anti-GP75 CD89 mRNA construct comprises an anti-gp75 scFv derived from TA99 which is fused to the truncated CD89 having the transmembrane and cytoplasmic domains. Efficacy of cancer antigen targeting myeloid CAR in surrogate mouse B16/F10-Ova melanoma tumors (subcutaneous, SC) in C57BL/6 mice was studied. The mouse model is a surrogate B16/F10 syngeneic animal model. In this surrogate model, GP75+B16/F10 melanoma tumor were developed and treated with anti-GP75 CAR mRNA in an LNP composition. The anti-GFP-CAR is structurally similar to an anti-TROP2 CAR except that the extracellular antigen binding domain is an scFv that binds to antigen GP75 instead of an scFv that binds to TROP2 antigen. The generalized structure is depicted in FIG. 1. C57BL/6 mice (n=5/group) were inoculated with 2× 105 GP75+B16/F10-OVA cells/mouse in the right flank on Day 0. In all experiments, treatments were initiated when tumors reached volumes of >30 mm3. The anti-GP75 CAR mRNA-LNP was injected intravenously (IV) into immunocompetent C57BL/6 mice bearing GP75+B16/F10-Ova melanoma tumors ranging from 4 days apart (Q4D) to 1 week apart (Q7D) and tumor volume was monitored. Mice received Vehicle Control, LNP loaded with GP75-CD89 mRNA at doses of 0.5 or 2 mg/kg/dose, by IV injection, (data are shown in FIG. 2) at days 8, 12 and 16 post inoculation of the tumor. Both 0.5 mg/kg and 2 mg/kg showed statistically significant slower/stalled tumor growth compared to vehicle. This shows that the CAR had anti-tumor activity, and can slow and even stall tumor progression.

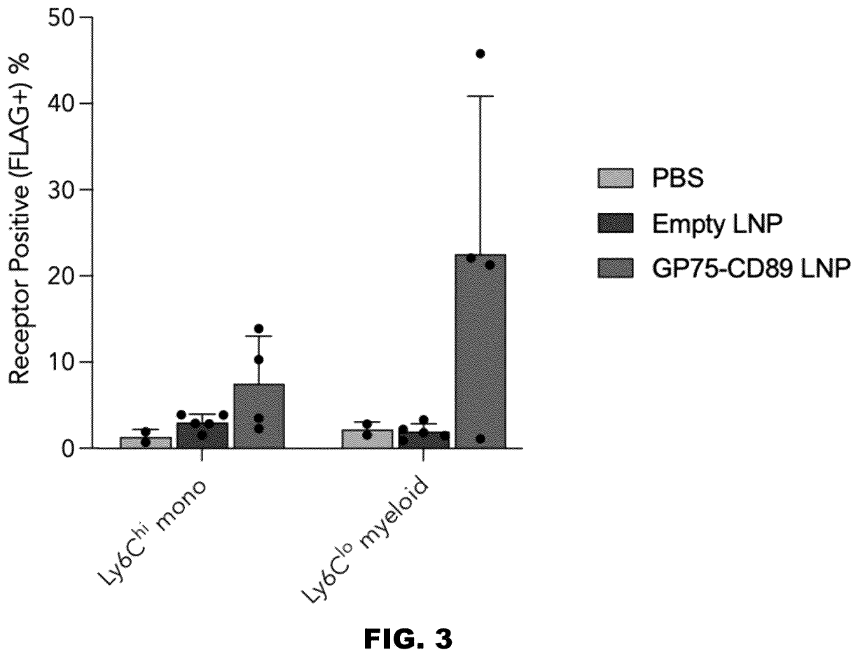

In one study, C57BL/6 mice bearing tumors were treated with Vehicle (PBS), Empty LNP (2 mg/kg) or GP75-CD89 LNP (2 mg/kg) on Days 9, 11, 13, and 15. Tumors were harvested 24 hours after the 4th dose. Single cell suspension was obtained from tumor and CAR expression was evaluated by FACS using anti-FLAG Ab, in CD45+ CD11b+ Ly6Chi (monocytes) and in CD45+ CD11b+ Ly6C^{lo} (resident myeloid cells) populations. Error bars represent standard error of the mean. Statistical significance was established by ordinary two-way ANOVA vs Empty LNP. For mice treated with 2 mg/kg/dose gp75 LNP, anti-GP75 CAR expression was detected in up to 15% of the CD11b+ Ly6C+ cells in tumors (FIG. 3).

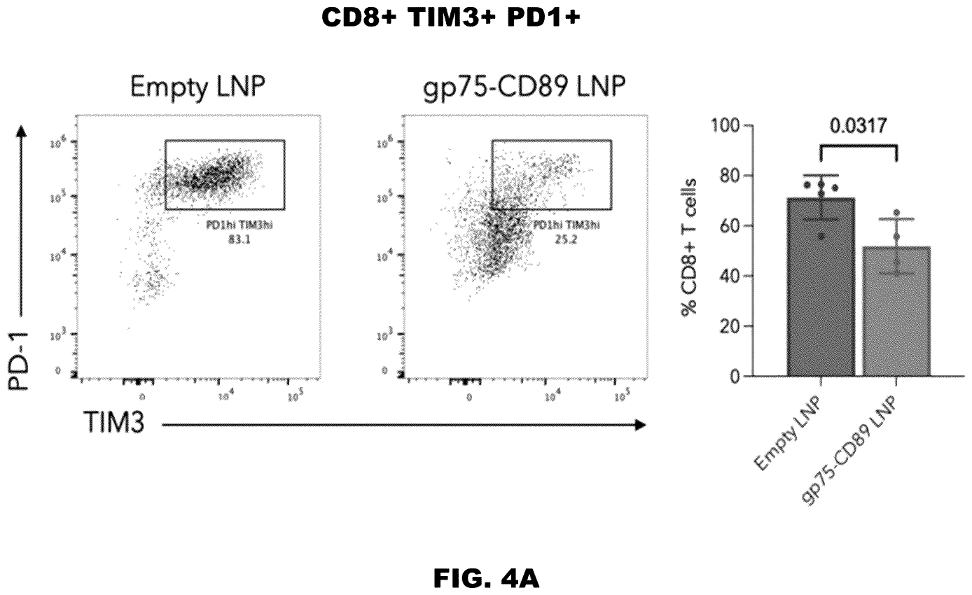

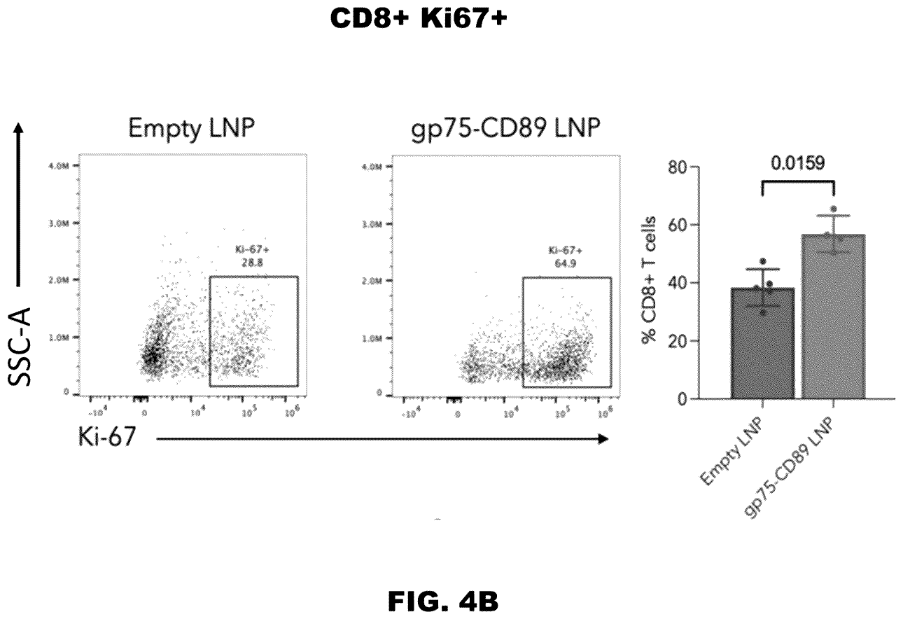

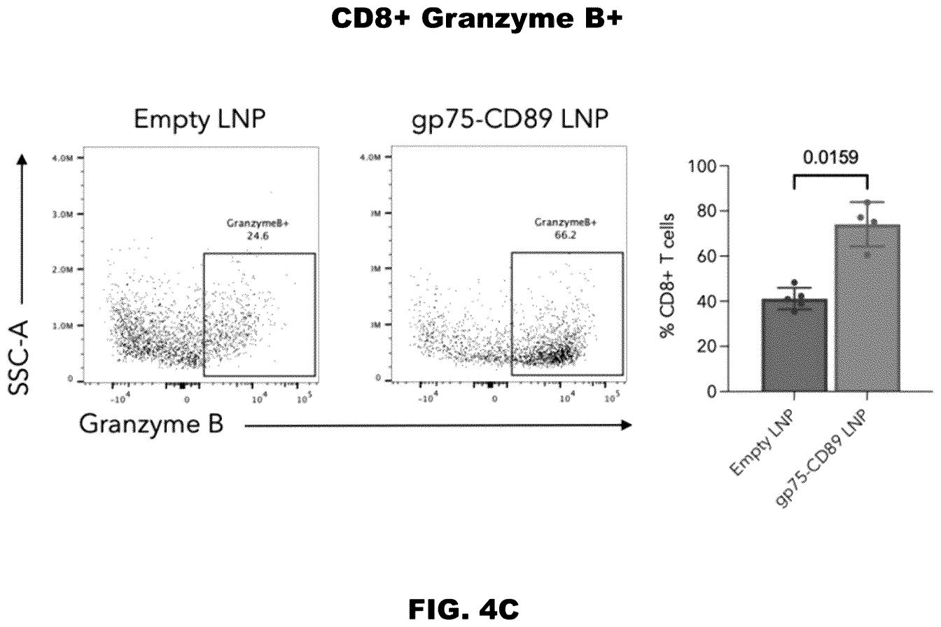

C57BL/6 mice bearing tumors were treated with Vehicle (PBS), Empty LNP (2 mg/kg) or gp75-CD89 LNP (2 mg/kg) on Days 9, 11, 13, and 15. Tumors were harvested 24 hours after the 4th dose. Single cell suspension was obtained from tumor. FIG. 4A, Dot plots and bar graph depicting activation and exhaustion (TIM3/PD-1), FIG. 4B, Dot plots and bar graph depicting proliferation (Ki-67), FIG. 4C, Dot plots and bar graph depicting cytolytic activity (Granzyme B). Dots in bar graphs represent individual animals. Error bars represent standard error of the mean. Statistical significance was established using a Mann-Whitney test by comparing Empty LNP with gp75-CD89 LNP. Treatment was associated with an increase in proliferating (Ki-67+) (FIG. 4B) and cytolytic (granzyme B+) (FIG. 4C). CD8+ T cells exhibiting an activated phenotype (with reduction in PD-1 expression level) (FIG. 4A). Dots on bar graphs of FIGS. 4A-4C represent individual animals. Error bars represent standard error of the mean. Statistical significance was established using a Mann-Whitney test by comparing Empty LNP with gp75-CD89 LNP.

Serum analysis revealed increases in chemokines and cytokines associated with the induction of an adaptive immune response, including CXCL10 (IP-10), CCL5 (RANTES), CCL2 (MCP-1), CCL7 (MCP-3), CCL3 (MIP-1-α), CCL4 (MIP-1-β), CXCL2 (MIP-2), TNF-α, GM CSF, IFN-γ and IL-17a, which were most pronounced at 2 mg/kg.

### Example 4. Non-Clinical Studies: Data from Mouse Triple-Negative Breast Cancer Model

This study shows the activity of Test composition (Anti-TROP2-CD89 mRNA encapsulated in LNP) against human TROP2-expressing tumors in a xenograft model in immune deficient mice. The objectives of these studies were to evaluate the anti-tumor efficacy of Test composition at different dosing regimens in the TROP2+ HCC-1954 human epithelial breast cancer tumor xenograft model in NSG and NCG mice. Because these animals do not have a fully replete immune system, these studies address only the direct anti-tumor activity of programmed myeloid cells. The investigation of other downstream immune elements is not possible. Contrary to syngeneic models where other immune cells, and particularly the adaptive compartment (T and B cells) can contribute to the anti-tumor effect following activation by CAR myeloid cells, clinical efficacy in xenograft models solely relies on CAR myeloid cells, which need to be constantly provided to the tumor site. Despite this caveat, treatment of NSG and NCG mice harboring the TROP2 expressing HC-1954 xenograft with Test composition was associated with significant anti-tumor activity.

NSG or NCG mice (n=5/group) were inoculated with 2×10\hat{ }6 TROP2+ HCC-1954 cells (triple negative breast cancer) per mouse, via subcutaneous (SC) injection in the right flank on Day 0. When tumor volume reached 50-100 mm³, mice received injections of Vehicle (DPBS), Empty LNP, or Test composition at doses of 0.1, 0.5, 1 or 2 mg/kg. Mice were injected intravenously (IV) following various dosing regimens including every 4 days (Q4D) for a total of 5 injections, weekly (QW) for a total of 3 or 4 injections, or every other week (Q2W) for a total of 4 injections.

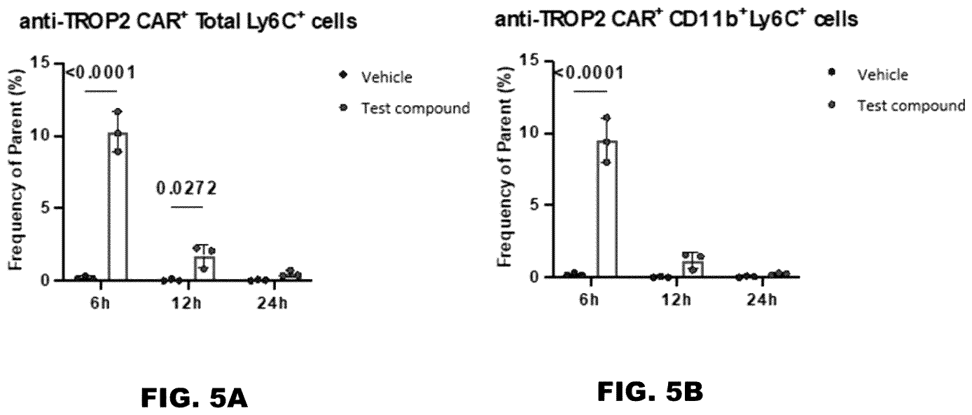

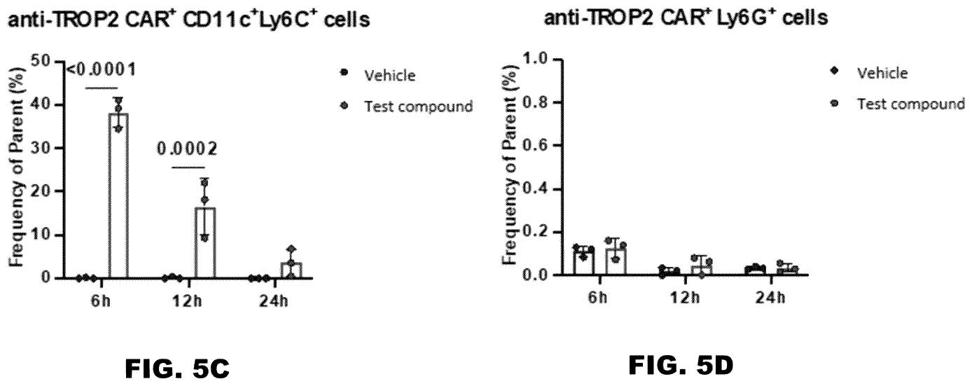

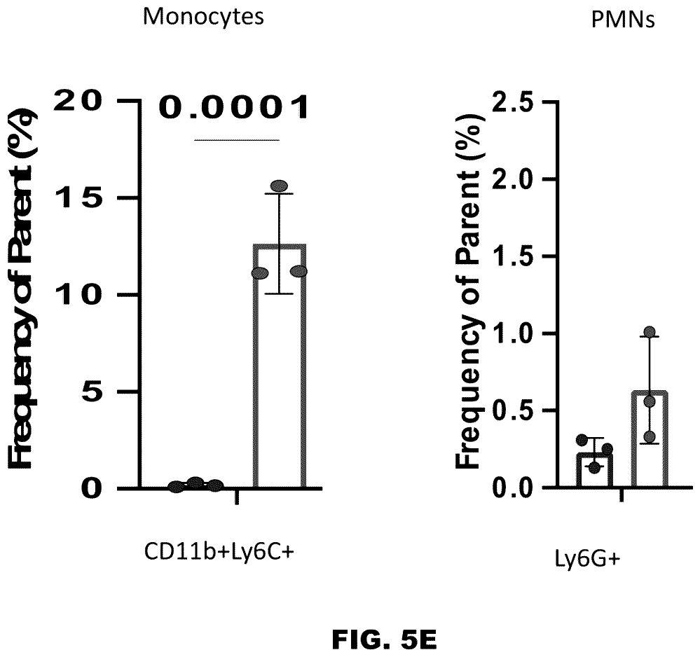

Examination of CFP expression was conducted on various cell populations (Ly6C+, Ly6G+) from blood, spleen, bone marrow and tumor in tumor-bearing animals at 6, 12 and 24 hours after Test composition infusion. CFP expression was restricted to cells of the myeloid lineage, especially Ly6C+ (monocytes), as soon as 6 hours post infusion, in all tissues examined (e.g., whole blood). FIGS. 5A-5D depict expression of anti-TROP2 CFP in myeloid cells from whole blood following intravenous infusion of Test composition in a TROP2+ HCC-1954 subcutaneous xenograft model in NCG Mice. Expression was evaluated by FACS. FIG. 5A depicts the total Ly6C+ cells; FIG. 5B depicts Ly6C+ CD11b+ cells; FIG. 5C depicts Ly6C+ CD11c+ cells; and FIG. 5D depicts Ly6G+ cells. Statistical significance was determined for Test composition vs Vehicle treated mice (n=3/group/time point) at each time point using an Ordinary two-way ANOVA followed by Sidak's multiple comparisons test. Even with myeloid cell migration patterns and phenotypical changes, CFP expression was detected in the blood and spleen for up to 24 hours. FIG. 5E shows a set of results in concurrence with the above, where expression of the CAR was found in monocytes and DCs. Expression was depicted as % of total number of isolated cells having the specific marker described in the figure, that are positive for the CAR expression. The data shows that the CAR is expressed across myeloid subsets.

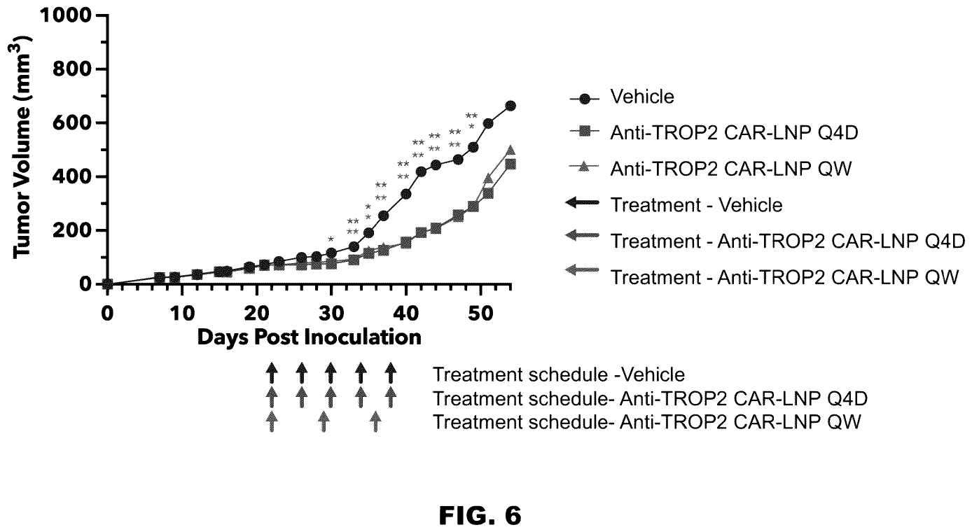

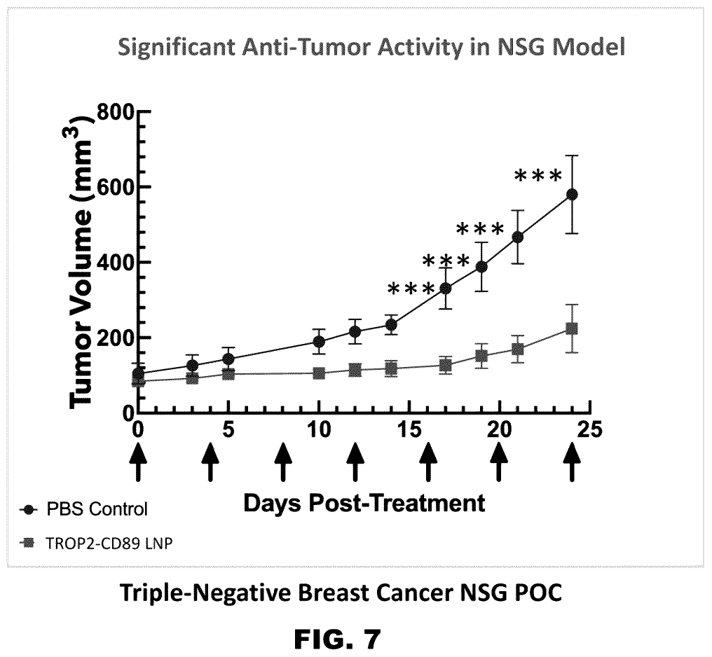

Anti-tumor efficacy was dose-dependent with anti-tumor activity evident in animals treated with Test composition ≥0.5 mg/kg/dose compared to Vehicle and to Empty LNP controls. A significant anti-tumor efficacy was observed with Test composition at 2 mg/kg when administered IV Q4D and was observed for up to 11 days following interruption of treatment. FIG. 6 depicts mean tumor volume per group following once every four days (Q4D) or once weekly intravenous administration of the Test composition in a TROP2⁺ HCC-1954 subcutaneous xenograft model in NSG mice. Vehicle (DPBS) or Test composition (2 mg/kg) was administered IV once every four days (Q4D), or weekly (QW), starting 22 days post inoculation. Statistical significance was determined using an Mann-Whitney tests followed by 2-stage false discovery rate approach. * p<0.05; ** p<0.01; *** p<0.001. Red and Green asterisks define statistical significance of Test composition Q4D and QW treatment compared to Vehicle, respectively. Significant efficacy was also observed with Test composition when administered IV QW or Q2W at 0.5 mg/kg and 1 mg/kg. Treatment with Empty LNP was not associated with any anti-tumor efficacy, confirming the antigen-specificity of the anti-tumor effect. Treatment appeared to be well-tolerated with no visible adverse events reported, no body weight loss, and no relevant change in body condition noted in mice treated with Test composition. Similar results consistent with the above is shown the study depicted in FIG. 7, where the dosing schedule of the test composition after tumor inoculation is shown in arrows.

### Example 5. Non-Clinical Studies: Non-Human Primate Studies

Adult primates (cynomolgus monkeys) express TROP2. The following studies were undertaken to evaluate the presence and specificity of the anti-TROP2 CFP in nonhuman primate blood leukocytes after Test composition infusion. Flow cytometry was used to evaluate CD66abce+ granulocytes, CD14+ CD11b+ monocytes, CD11b+ CD14− myeloid cells, CD3+ T cells, CD20+ B cells, and CD16+ NK cells in the blood of cynomolgus monkeys treated with Test composition.

The results were consistent across studies, demonstrating the presence of anti-TROP2 CAR+ myeloid cell populations, including monocytes, CD11b+ myeloid cells, and granulocytes following the 1st IV infusion of Test composition (Dose 1). The number of CAR positive cells in the blood was dose dependent, with the highest expression observed in the monocyte population 12 hours after IV administration of Test composition at 1 mg/kg/day. The number of CFP positive cells was also significantly elevated after IV administration of Test composition at 0.5 mg/kg/day. The number of CFP positive cells in the blood peaked 12 hours after the first infusion of Test composition in all myeloid cell types including monocytes, CD11b+ myeloid cells, and granulocytes, and was reduced at 24 hours post-infusion in monocytes (Mean 0.92%+/−SD 1.98%), in CD11b+ myeloid cells (Mean 0.78%+/−SD 1.16%), and in granulocytes (Mean 0.48%+/−SD 0.84%). In CD11b+ myeloid cells and granulocytes, CAR expression was only slightly above background at 24 hours post Test composition infusion. No significant CFP expression was observed in non-myeloid cell populations including T cells, B cells, and NK cells. The anti TROP2 CAR expression profile was similar between males and females in all tested cell types.

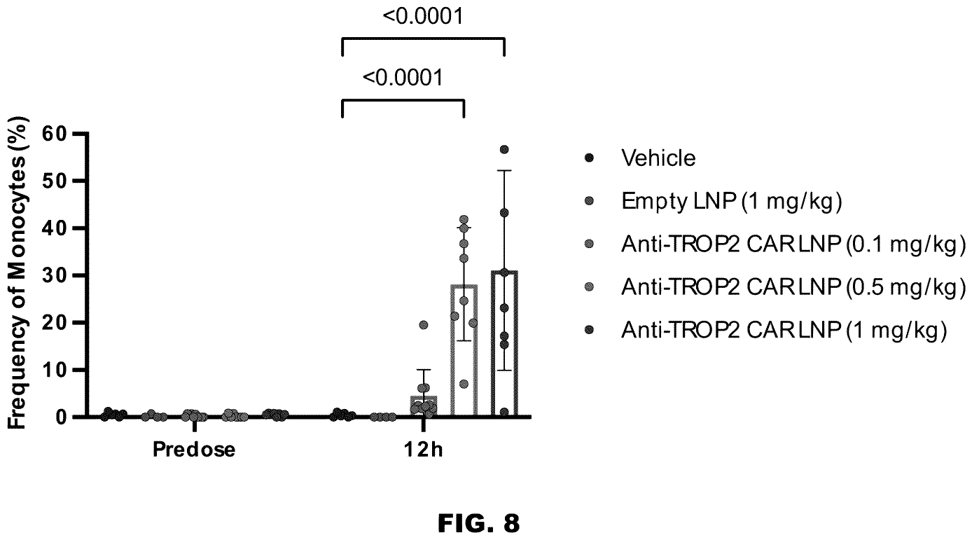

FIG. 8 depicts the frequency of monocytes expressing anti-TROP2 CFPs in whole blood after intravenous infusion of Test composition in Cynomolgus monkeys. Cynomolgus monkeys were administered with Vehicle, Empty LNP at 1 mg/kg/dose, or Test composition at 0.1, 0.5, or 1 mg/kg/dose, via IV infusion. The frequency of anti-TROP2 CAR was evaluated by flow cytometry at 12 h following the first infusion. Statistical analysis was performed by 2-way ANOVA followed by Dunnett Multiple comparison test, by comparing each treatment group to Vehicle.

The effect of Test composition on the central nervous and cardiovascular systems was evaluated in cynomolgus monkeys. Groups of 4 (2 males, 2 females) to 10 (5 males, 5 females) cynomolgus monkeys were administered Vehicle, Empty LNP at 1.0 mg/kg/dose, or Test composition at 0.1 mg/kg/dose (Days 1, 8, 15 and 29, 36, 43), or Test composition at 0.5 or 1.0 mg/kg/dose (Days 1, 8, 15 and 29), via a 60-minute IV infusion. See Table 7.

There were no Test composition related effects on neurological examinations performed on Day 8 following a 60-minute IV infusion of 0.1, 0.5, or 1.0 mg/kg/dose of Test composition, or on Day 36 following a 60-minute IV infusion of 0.1 mg/kg/dose of Test composition, in cynomolgus monkeys. There were also no test-compound-related effects on electrocardiology (ECG) examinations on Day 43 following a 60-minute IV infusion administration of 0.1 mg/kg/dose of Test composition in cynomolgus monkeys, or in any dose group following a 30-day recovery period.

In the studies described below, the anti-TROP2-CD89 CAR: LNP was used to assess the distribution in a model where on-target-off-tumor activities of the drug could be monitored. Distribution of mRNA was performed in highly perfused tissues (liver, lungs, heart, brain, kidneys, ovaries/testes) from cynomolgus monkeys in the recovery arm of the GLP toxicological study 30 days following administration of the drug. The no observed adverse effect level (NOAEL) in non-human primates was 0.1 mg/kg.

Pharmacokinetic studies in non-human primates indicated that observed half-lives of the two lipids are approximately 8-10 hours and 35-55 hours, respectively for Cohort 1. Thus, the Q14D dosing interval allows for complete drug product clearance prior to the repeat doses. The hypersensitivity reactions observed in NHPs were not predictive of hypersensitivity reactions in patients.

A human equivalent dose (HED) at which hypersensitivity reactions were observed in the NHP toxicology studies is 0.16 mg/kg.

Secondary Pharmacodynamics and Studies from Similar Pharmacological Product

In vivo safety pharmacology was tested, the effect of anti-TROP2-CD89 CAR: LNP on the central nervous and cardiovascular systems were evaluated as part of the GLP-compliant, repeat-dose toxicology study conducted in groups of 4 (2 males [M], 2 females [F]) to 10 (5M, 5F) cynomolgus monkeys that were administered empty LNP at 1 mg/kg/dose, or anti-TROP2-CD89 CAR: LNP at 0.1 mg/kg/dose (Days 1, 8, 15 and 29, 36, 43), or anti-TROP2 CAR: LNP at 0.5 or 1.0 mg/kg/dose or 2.0 mg/kg/dose (Days 1, 8, 15 and 22 or 29 or as indicated), via a 60-minute IV infusion. The results are provided in Table 7.

| | | | |
|---|---|---|---|
| | | Gender, | |
| | Dose/Route; | Number per | |
| Test System/Model | Frequency | group | Findings |

Absorption

| | | | |
|---|---|---|---|
| 21-day intravenous | Vehicle (saline); | 1 to 2 | Peak concentrations of the |
| infusion toxicity study | Empty LNP at 2.0 | M/group, | lipids were attained at the EOI, |
| with toxicokinetic of | mg/kg/dose; or anti- | 1 to 2 | thereafter concentrations |
| lipid nanoparticle | TROP2 CAR:LNP at | F/group | declined and were still |
| formulated or anti- | 0.5, 1, or 2 | | quantifiable 144 hours after the |
| TROP2 CAR:LNP | mg/kg/dose; 60- | | end of the infusion. Overall, |
| drug product in non- | minute IV infusion | | systemic exposure to PEG and |
| naïve cynomolgus | Days 1, 8, 15, and 22 | | ionizing lipid was increased in |
| monkeys with a 14- | | | a general dose proportional |
| day recovery period | | | manner across the dose range |
| | | | in both sexes. No sex |
| | | | differences were apparent |
| | | | across doses on Day 1 or Day |
| | | | 22. |
| 43-day study of anti- | Vehicle (saline); | 2 to 5 | Peak concentrations of Lipid 1 |
| TROP2 CAR:LNP | Empty LNP 1.0 | M/group, | and Lipid 2 were |
| (Anti-TROP2 CAR | mg/kg/dose; or anti- | 2 to 5 | attained at the EOI, thereafter |
| LNP) by intravenous | TROP2 CAR:LNP at | F/group | concentrations declined and |
| infusion in | 0.1 mg/kg/dose; 60- | | were still quantifiable 144 |
| cynomolgus | minute IV infusion | | hours after the end of the |
| nonhuman | Days 1, 8, 15, 29, | | infusion. |
| primate with a 30-day | 36, 43 anti-TROP2 | | Groups 3 recovery animals had |
| recovery period - lipid | CAR:LNP at 0.5 or | | measurable concentrations |
| toxicokinetic | 1.0 mg/kg/dose; 60- | | of Lipid 1 on Day 73 close to |
| parameters | min IV | | the limit of quantification (~2-3 |
| | infusion Days 1, 8, 15, | | fold). Recovery animals from |
| | and 29 | | Group 5 (1 mg/kg) had |
| | | | measurable concentrations of |
| | | | Lipid 1 and Lipid 2 on Day |
| | | | 29/30 following the final dose. |
| | | | Group 4 (0.5 mg/kg) and Group |
| | | | 5 (1 mg/kg) recovery animals |
| | | | still showed exposure on Day |
| | | | 59 |

Distribution

| | | | |
|---|---|---|---|
| 43-day study of anti- | Vehicle (saline); | 2 to | Following a 30-day dose-free |
| TROP2 CAR:LNP by | Empty LNP 1.0 | 5M/group, | recovery period, the anti- |
| IV infusion in | mg/kg/dose; or anti- | 2 to 5F/group | TROP2 CAR:LNP mRNA drug |
| cynomolgus | TROP2 CAR:LNP at | | substance was below the RT- |
| nonhuman primate | 0.1 mg/kg/dose; 60- | | qPCR detection limit (<0.001 |
| with a 30-day recovery - | min IV infusion Days | | mg/kg) in the evaluated tissues |
| biodistribution of | 1, 8, 15, 29, 36, 43; | | (liver, kidney, lung, heart, |
| anti-TROP2 | anti-TROP2 | | brain, blood cells, testis, and |
| CAR:LNP mRNA | CAR:LNP at 0.5 or | | ovary) |
| drug substance by RT | 1.0 mg/kg/dose; 60- | | |
| qPCR limit test | min IV infusion Days | | |
| | 1, 8, 15, 29 | | |

Excretion

| | | | |
|---|---|---|---|
| 43-day study of anti- | Vehicle (saline); | 2 to 5 | In monkey excreta, there was |
| TROP2 CAR:LNP | Empty LNP 1.0 | M/group, | no detectable Lipid 2 in urine at |
| (Anti-TROP2 CAR | mg/kg/dose; or anti- | 2 to 5 | any time points. Lipid 2 |
| LNP) by intravenous | TROP2 CAR:LNP at | F/group | concentrations in monkey feces |
| infusion in | 0.1 mg/kg/dose; 60- | | were minimal and the |
| cynomolgus | min IV infusion Days | | cumulative percentage of a |
| nonhuman primate | 1, 8, 15, 29, 36, 43; | | lipid excreted unchanged in |
| with a 30-day recovery | anti-TROP2 | | feces was ~1.1%. |
| period - urine lipid | CAR:LNP at 0.5 or | | |
| levels | 1.0 mg/kg/dose; 60- | | |
| | min IV infusion Days | | |
| | 1, 8, 15, 29 | | |

### Example 6. In Vitro Expression of Anti-TROP2 CAR

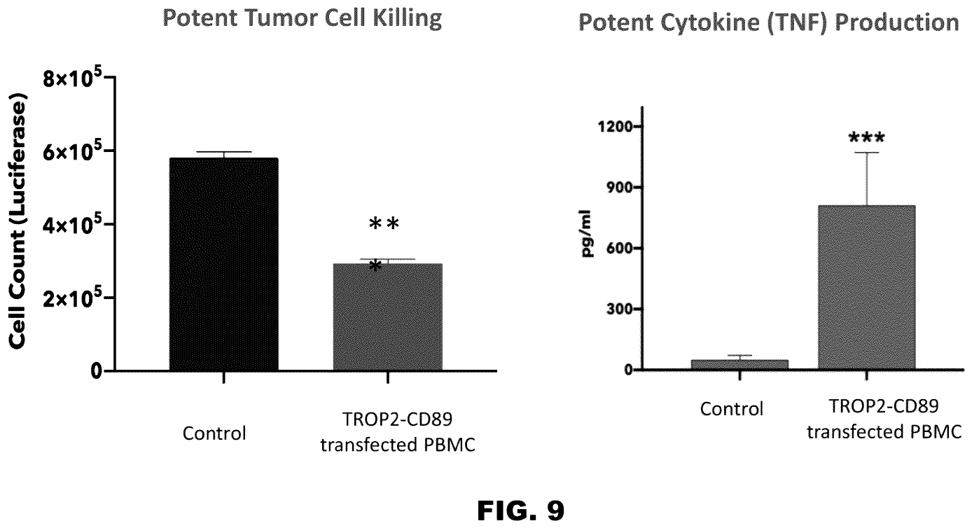

Direct tumor-cell killing and cytokine production was assessed in TROP2-CD89 CAR construct transfected PBMCs. These cells showed elevated TROP2-positive tumor cell killing when brought in contact (FIG. 9, left bar diagram). These cells also demonstrated high cytokine levels, shown in FIG. 9, right, depicting TNF production.

### Example 7. First-In-Human Phase I Study Design and Progress

This example describes a Phase 1, open-label, non-randomized, first-in-human, dose escalation study to investigate the safety, tolerability, pharmacokinetics (PK), pharmacodynamics (PD), and preliminary efficacy of a Test composition in adults with advanced or metastatic epithelial tumors. This is a multicenter study that includes previously treated patients with metastatic epithelial cancers including: urothelial, cervical, ovarian epithelial, Triple-negative breast, HR+/HER2− breast, pancreatic ductal adenocarcinoma, gastric adenocarcinoma, esophageal carcinoma, Non-small cell lung, and colorectal cancer.

The study was designed as follows: Adult patients 18 years of age inclusive or older will be screened. Patients who provide written informed consent and meet all inclusion and exclusion criteria will be entered into the trial.

Screening evaluations 28 days (4 weeks) prior to the scheduled start of treatment include participant history, physical examination with vital signs and performance status, computed tomography (CT) or magnetic resonance imaging (MRI) scans, complete blood count (CBC) with differential and platelet count, routine serum chemistries, urinalysis, International normalized ratio (INR)/PTT, EKG, and serum samples for human anti-human antibody (for both anti-TROP2 CAR and anti-PEG antibodies). In women of childbearing potential, serum β-HCG is also re-quired within one week of treatment.

The study consists of 4 Cycles. For Cohorts 1-3, during Cycle 1, all participants will receive infusions of Test compound every 14 days for 3 doses. For Cohort 4, all participants will receive infusions of Test compound every 7 days for 3 doses. Participants will continue in the study in the absence of unacceptable toxicity through Week 18, but the dose regimen will be modified to once every 28 days for Cycles 2-4 for all Cohorts. If a participant has stable disease (SD), or a partial response (PR), the participant will continue monthly dosing through Week 48 until progressive disease or a complete response. Once a participant discontinues dosing, follow-up will continue, with evaluations performed every 3 months until the end of the study or until progression of disease.

All patients should be closely monitored over the course of their treatment and to provide dose reduction, delay, or cessation guidelines in the event of treatment-related toxicity. NCI CTCAE version 5.0 will be used to grade all adverse events. All patients will also have CT/MRI scans throughout the study to assess for progression of disease as well as response to study drug.

A baseline tumor biopsy will be obtained during screening if no archived biopsy (<6 months) is available. Biopsies at Week 12 and EOT are optional for Cohorts 1 and 2. Biopsies for Cohorts 3 and 4 are required. The biopsy will be used to determine if the myeloid cells programed by Test compound have migrated to the tumor and to assess the change in the tumor microenvironment (including infiltration of T- and NK cells).

Whole blood and serum will be obtained for anti-drug antibodies (ADA), cell phenotype, cell kinetics, and TCR sequencing.

TABLE 8
Summary of Dose Escalation and Study Cohorts

| | Dose | |
|---|---|---|
| | (mg/kg/ | |
| Cohort | dose) | Dose Regimen |
| Cohort 1 (n = 3, up to 12) | 0.0075 | Cycle 1: Dosing every 14 days |
| Cohort 2 (n = 3, up to 12) | 0.015 | Cycles 2-4: Dosing every 28 days |
| Cohort 3 (n = 3, up to 12) | 0.03 | |
| Cohort (n = 3, up to 12) | 0.03 | Cycle 1: Dosing every 7 days |
| | | Cycles 2-4: Dosing every 28 days |

FIG. 10A summarizes this part of the clinical study design. For Cohorts 1-3, the dosing regimen in the human study will be every 14 days for 3 doses, followed by administration once every 28 days for three doses. For Cohort 4, the dosing regimen will be modified such that participants will receive one dose of Test compound every week for 3 doses, followed by administration once every 28 days for three additional doses.

This example describes results obtained thus far from the ongoing Phase I, open-label first in human dose escalation study to investigate the safety pharmacokinetics, pharmacodynamics and preliminary efficacy of anti-TROP2-CD89 CAR mRNA-LNP drug in adults with advanced or metastatic epithelial tumors described. As of May 2024, nine patients have been dosed up to 0.03 mg/kg of the drug with no dose limiting toxicites (DLT). In this study, Cohorts 1 (0.005 mg/kg) and 2 (0.015 mg/kg) have been completed and Cohort 3 (0.03 mg/kg) was completed at the time of preparing the instant application and was reportedly well tolerated. Therefore the results were highly encouraging.

### Example 8. First-In-Human Phase I Study Dose Escalation Update: a Phase 1, Open-Label, First-In-Human, Dose Escalation Study to Investigate the Safety, Pharmacokinetics, Pharmacodynamics and Preliminary Efficacy of the Anti-TROP2-CD89 CAR mRNA-LNP in Adults with Advanced or Metastatic Epithelial Tumors

This example describes an advancement to the FIH study in which another cohort wherein a higher dose is included (FIG. 10B). The starting dose is based on the Highest Non-severely Toxic Dose (HNSTD) which is equivalent to the No Observed Adverse Effect Level (NOAEL) in the nonhuman primates (NHP) safety studies. The HNSTD in NHPs was 0.1 mg/kg. The Human Equivalent Dose (HED) for 0.1 mg/kg in NHPs is 0.032 mg/kg using body surface area-based conversion. For this calculation, 0.1 mg/kg in NHPs is multiplied by the scaling factor of 0.32 to arrive at the HED in mg/kg. The initial dose of 0.005 mg/kg in Cohort 1 is 6 times below the HED of the HNSTD in the NHP safety study, and there will be 3 dose levels (Table 9).

For Cohorts 1-3, the dosing regimen in the human study was every 14 days (Q14D) for 3 doses, followed by administration once every 28 days (Q28D) for three doses. For Cohort 4, the dosing regimen is modified. Participants will receive one dose of anti-TROP2-CD89 CAR mRNA-LNP every week for 3 doses (Q7D), followed by administration once every 28 days (Q28D) for three additional doses. The initial doses will create an innate immune response, allowing short term control of tumor growth; all six doses will initiate and boost an adaptive immune response, allowing long-term control of tumor growth.

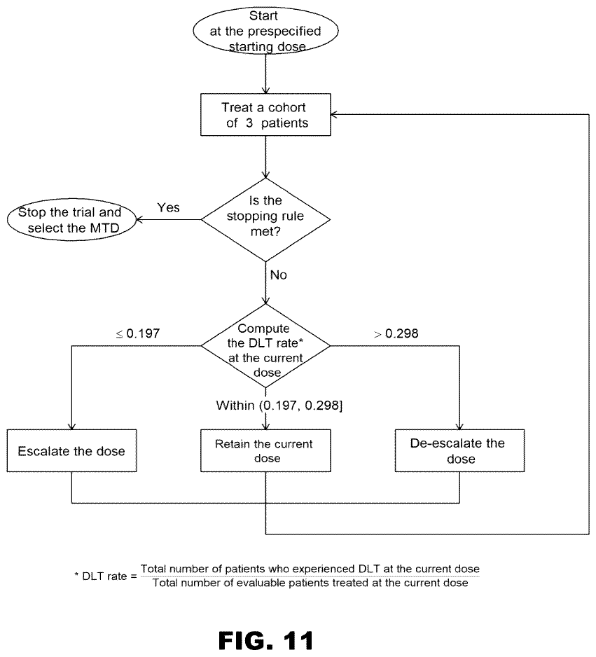

To advance the safety and efficacy evaluation of anti-TROP2-CD89 CAR mRNA-LNP, this protocol will further dose escalate above the HED (0.032 mg/kg) of the NOAEL found in NHPs. Specifically, doses of 0.06 mg/kg, 0.10 mg/kg, and 0.15 mg/kg are planned; all of these doses are below the HED (0.16 mg/kg) at which hypersensitivity reactions were observed in the NHP toxicology studies (Example 5). Justification for these additional dose levels may be based on the initial safety profile observed at doses up to 0.03 mg/kg. Continued enhanced monitoring for potential hypersensitivity reactions and the addition of hypersensitivity treatment algorithms will be used to optimize patient safety. FIG. 11 is a schematic outline of the dosing and schedule.

In the anti-TROP2-CD89 CAR mRNA-LNP (DP) NHP toxicology studies, some animals receiving doses above the HED of 0.16 mg/kg experienced severe or fatal reactions. These reactions in NHP were characterized by a syndrome consistent with prior observed NHP type III hypersensitivity reactions and corroborated by complement activation and correlated with anti-drug antibodies (ADA) development in 75% of animals (See the IB for more information). Empty LNP (1 mg/kg) showed no toxicity. ADAs in the monkey were directed against the single-chain variable fragment (scFv) of the CAR. The correlation between the incidence rate of immunogenicity or immune reactions to human protein therapeutics in NHPs, and immunogenicity incidence/immune reactions in humans is limited (Vahle 2018, van Meer 2013). Immunogenicity-related risks in humans therefore cannot be reliably predicted from NHPs (i.e. immunogenicity-related risks observed with human therapeutics in NHPs typically do not translate to humans).

Cohorts 1 (0.005 mg/kg) and 2 (0.015 mg/kg) have been completed; enrollment in Cohort 3 (0.030 mg/kg) is ongoing as of May 1, 2024. There have been no dose-limiting toxicities (DLTs) or ADAs seen to date. All AEs assessed by the Investigator as related to the DP have been Grade 1, except for one transient Grade 2 diaphoresis reported in one patient (Cohort 2). Notably, a confirmed partial response at 0.015 mg/kg (per RECIST 1.1) was observed in a patient with hormone receptor positive breast cancer. In addition, peripheral changes in the cytokine milieu and T-cell receptor repertoire are indicative of potential biological activity of the drug development candidate. Collectively, the favorable safety profile and potential activity observed through May 1, 2024 support continued clinical investigation at higher doses.

For doses of 0.06 mg/kg to 0.15 mg/kg in cohorts 5-7, dosing frequency will be modified to one dose of the DP administered every 14 days (Q14D) for assessment of safety on a consistent dosing schedule (e.g., two doses per 28-day cycle). The proposed dosing schedule is supported by initial pharmacokinetics (PK) data from the ongoing anti-TROP2-CD89 CAR mRNA-LNP study. The observed half-lives of the ionizable and pegylated lipids are approximately 8-10 hours and 35-55 hours, respectively, for Cohort 1. Clearance of the lipids prior to the repeat doses is in line with data collected in the anti-TROP2-CD89 CAR mRNA-LNP GLP NHP toxicology studies and hence supports the Q14D dosing intervals.

During the proposed further dose escalation, patients will be closely monitored for hypersensitivity reactions and emergent symptoms will be treated promptly as they arise based upon appropriate investigator, care team, and participant disclosure and education. The ongoing comprehensive evaluation of inflammatory markers and ADAs will continue, as well as a 16-day sentinel period of observation for the first patient treated at each dose level which allows a staggered safety evaluation of repeat dosing across patients.

The proposed measured dose evaluation and patient management approach is supported by experts who reviewed NHP hypersensitivity data in the setting of predicting risk in humans (Vahle 2018).

TABLE 9
Summary of Dose Escalation and Study Cohorts

| | Dose | |
|---|---|---|
| Cohort | (mg/kg) | Dose Regimen |
| Cohort 1 | 0.005 | Cycle 1: Dosing every 14 days (Q14D) |
| (n = 3, up to 12) | | Cycles 2-4: Dosing every 28 days (Q28D) |
| Cohort 2 | 0.015 | |
| (n = 3, up to 12) | | |
| Cohort 3 | 0.03 | |
| (n = 3, up to 12) | | |
| Cohort 4 | 0.03 | Cycle 1: Dosing every 7 days (Q7D) |
| (n = 3, up to 12) | | Cycles 2-4: Dosing every 28 days (Q28D) |
| Cohort 5 | 0.06 | All Cycles: Dosing every 14 days (Q14D) |
| (n = 3, up to 12) | | |
| Cohort 6 | 0.10 | |
| (n = 3, up to 12) | | |
| Cohort 7 | 0.15 | |
| (n = 3, up to 12) | | |

### Updated Overall Study Design

This is a Phase 1, first-in-human, non-randomized, open-label, multicenter, dose-escalation study evaluating the safety, tolerability, PK, and pharmacodynamics of anti-TROP2-CD89 CAR mRNA-LNP.

Some exemplary terms used in abbreviated form herein and throughout the document may be tabulated below:

TABLE 10
List of non-exclusive abbreviations and definition of terms

| | |
|---|---|
| ADA | Anti-drug antibodies |
| ADC | Antibody-drug conjugate |
| AE(s) | Adverse event(s) |
| ALT | Alanine aminotransferase |
| AST | Aspartate transaminase |
| ASTCT | American Society for Transplantation and Cellular Therapy |
| AUC | Area under the curve |
| BOIN | Bayesian Optimal Interval |
| C#D# | Cycle number/Dose number (eg, Cycle 1 Dose 1 = C 1 D 1) |
| CAR | Chimeric antigen receptor |
| CBC | Complete blood count |
| CFR | Code of Federal Regulations |
| cfDNA | Circulating free DNA |
| CKD-EPI | Chronic Kidney Disease Epidemiology Collaboration |
| CL | Plasma clearance |
| Cₘₐₓ | Maximum observed plasma concentration |
| CNS | Central nervous system |
| CPK | Creatine phosphokinase |
| CR | Complete response |
| CRP | C-reactive protein |
| CRS | Cytokine release syndrome |
| CT | Computed tomography |
| CTCAE | Common Terminology Criteria for Adverse Events |
| DLT | Dose limiting toxicities |
| DOR | Duration of response |
| ECG | Electrocardiogram |
| ECHO | Echocardiogram |
| ECOG | Eastern Cooperative Oncology Group |
| eCRF | Electronic case report form |
| EDC | Electronic data capture |
| EOT | End of treatment |
| Fc | Fragment crystallizable |
| FcRγc | Fc receptor common γ chain |
| FDA | Food and Drug Administration |
| FSH | Follicle stimulating hormone |
| GCP | Good Clinical Practice |
| HED | Human equivalent dose |
| HNSTD | Highest non-severely toxic dose |
| IB | Investigator brochure |
| ICANS | Immune effector cell-associated neurotoxicity syndrome |
| ICE | Immune effector cell-associated encephalopathy |
| ICF | Informed consent form |
| ICH | International Council for Harmonisation |
| IEC | Independent ethics committee |
| IND | Investigational new drug |
| INR | International normalized ratio |
| IP | Investigational product |
| IR | Infusion reaction |
| IRB | Institutional review board |
| MRI | Magnetic Resonance Imaging |
| MTD | Maximum tolerated dose |
| mTNBC | Metastatic triple negative breast cancer |
| MUGA | Multigated acquisition |
| NCI | National Cancer Institute |
| NOAEL | No observed adverse effect level |
| NT | Neurologic toxicity |
| ORR | Objective response rate |
| OS | Overall survival |
| PD | Progressive disease |
| PEG | Polyethylene glycol |
| PFS | Progression-free survival |
| PK | Pharmacokinetics |
| PO | Per oral |
| PR | Partial response |
| PT | Prothrombin time |
| PTT | Partial thromplastin time |
| RECIST | Response Evaluation Criteria in Solid Tumors |
| RP2D | Recommended Phase 2 dose |
| SAE(s) | Serious adverse event(s) |
| SAP | Statistical analysis plan |
| scFv | Single-chain variable fragment |
| SD | Stable disease |
| SoD | Sum of diameters |
| SOP | Standard operating procedures |
| SRC | Safety Review Committee |
| t_{1/2} | Apparent terminal half-life |
| TCR | T-cell receptor |
| TEAE | Treatment emergent adverse event |
| TSH | Thyroid stimulating hormone |
| ULN | Upper limit of normal |
| WBC | White blood cell |
| WOCBP | Women of childbearing potential |

Screening evaluations 28 days prior to the scheduled start of treatment include participant medical and medication history, physical examination with vital signs and performance status, computed tomography (CT) or magnetic resonance imaging (MRI) scans, complete blood count (CBC) with differential and platelet count, routine serum chemistries, urinalysis, International normalized ratio (INR)/partial thromboplastin time (PTT), and electrocardiogram (ECG), in women of childbearing potential (WOCBP), serum b-HCG is also required within one week of treatment.

Primary assessment of DLTs will include the first 30 days following the first dose of study drug. Assessment of efficacy and safety will continue until the participant leaves the study or at the end of the study, whichever comes first. The dosing interval may be increased in the setting of ongoing clinical benefit and in discussion with the Medical Monitor (e.g., one dose per 28-day cycle).

In the event of treatment termination due to unacceptable toxicity the participant may continue in the study until the occurrence of progression of disease or initiation of treatment of the primary malignancy, at which time an end-of-study evaluation will be performed, with additional follow-up required until resolution or stabilization of any treatment-related toxicity. All participants will be followed for overall survival.

To ensure the safety of study participants, the first two participants in each cohort will be staggered by an interval of 16 days. The 16-day period allows for a two-day follow-up after the third dose of study drug (for Cohort 4) or the second dose of study drug (for Cohorts 5-7). If there are no safety concerns, then participants may continue to enroll the cohort simultaneously. Cohorts 4 and 5 may enroll in parallel.

The BOIN design will be used to identify the recommended dose for expansion of the DP. Dose-escalation/de-escalation decisions will be based on events occurring during the DLT-Evaluation Period (30 days) and will be guided using a BOIN algorithm. Participants will be deemed evaluable for purposes of making dose-escalation decisions if they experience a DLT or complete the DLT-Evaluation Period without experiencing a DLT; participants who do not complete the DLT-Evaluation Period for reasons other than safety will not be considered evaluable.

The target toxicity probability of the MTD/RP2D of anti-TROP2-CD89 CAR mRNA-LNP in this study is set at a target toxicity rate of 25% (pT=0.25) and interval boundaries of [0.197, 0.298]. Doses with a toxicity probability between 0.197 and 0.298 may be deemed acceptable to be the MTD, except ⅓ DLT, under which the BOIN design retains the current dose.

To advance safety and efficacy evaluation of the DP, Protocol Version 3.0 seeks further dose escalation to include three additional doses. Justification for these dose levels is based on the safety profile observed in the first three cohorts. Intermediate dose levels and/or alternative dosing schedules may be evaluated per the SRC committee based on emerging clinical data.

Future modifications may include further escalation of doses above the NOAEL pending safety results, limit enrollment in expansion cohorts to a specific population such as breast cancer or non-small cell lung cancer, or the addition of a checkpoint inhibitor or other immune modifying drug product.

Participant population: Adult participants 18 years of age inclusive or older will be screened. Participants who provide written informed consent and meet all inclusion and exclusion criteria will be entered into the trial.

Dose limiting toxicity (DLT): A DLT may be understood using the National Cancer Institute (NCI) Common Terminology Criteria for Adverse Events (CTCAE) as specified. All toxicities will be considered “possibly” related to anti-TROP2-CD89 CAR mRNA-LNP unless they have no temporal association with the administration of the DP but rather related to other etiologies such as concomitant medications or conditions, or patient's underlying disease. For this study, the following are considered DLTs if they occur within 30 days of the first dose (Day 30):
Death
Any CTCAE Grade 4 toxicity
CTCAE Grade 3 toxicity in vital organs (central nervous system [CNS], heart and lung)
CTCAE Grade 3 toxicity that does not decrease to £ Grade 2 within 72 hours, except infection or renal/hepatic laboratory abnormalities.
CTCAE Grade 3 toxicity that does not decrease to £ Grade 2 within 7 days for infection or renal/hepatic laboratory abnormalities.
Any ³ Grade 3 infusion related reaction lasting >24 hours
Exceptions to DLT Grade 3 or 4 criteria include:

Laboratory values that are not considered clinically significant by the Investigator

Individuals may discontinue subsequent infusions upon Grade 4 toxicity after an infusion, or Grade 3 DLT, or Type III hypersensitivity reaction. A 50% reduced dose of the drug may be resumed after a Grade 3 DLT once toxicity resolves to Grade 1 or lower, and permanently discontinued if the DLT reoccurs.

### Sample Size

The estimated number of participants to be enrolled in this Phase 1 study evaluating anti-TROP2-CD89 CAR mRNA-LNP in participants with advanced solid tumors is approximately up to 42 in the dose escalation cohorts. At the Sponsor's discretion, an additional 18 subjects may be added to backfill cohorts. The number of participants per dose-escalation cohort has been determined based on a BOIN design. Participants are accrued into dosing cohorts in a cohort size of 3, and the number of participants at a dose level may expand to as many as 12 depending upon the number of participants with DLTs observed.

### Inclusion Criteria

Participants are eligible for the study if the following criteria are met:

Adults age ≥18 inclusive at the time the Informed Consent Form (ICF) is signed.

Histologically proven, metastatic or advanced epithelial cancer including the following cancer types: Urothelial, Cervical, Ovarian epithelial, Triple-negative breast, HR+/HER2− breast, Pancreatic ductal adenocarcinoma, Gastric adenocarcinoma, Esophageal carcinoma, Non-small cell lung, Colorectal

Other tumor types will be considered pending a discussion with the Medical Monitor.

Progressive disease at baseline, refractory or relapsed to standard of care or who have declined standard therapy.

Measurable disease based on Response Evaluation Criteria in Solid Tumors (RECIST) criteria v 1.1. Lesions situated in a previously irradiated area are considered measurable if unequivocal progression has been demonstrated in such lesions. Lesions intended to be biopsied should not be identified as the target lesion. If no measurable disease, evaluable disease will be considered in consultation with the Medical Monitor.
1. Eastern Cooperative Oncology Group (ECOG) performance status grade of 0 or 1.
2. Life expectancy of >12 weeks.
3. Echocardiogram (ECHO) or multiple gated acquisition scan showing an ejection fraction ³ 50%.
4. ECG showing no clinically significant abnormality at Screening or showing an average QTc interval <450 msec in males and <470 msec in females (<480 msec for participants with bundle branch block). Either Fridericia's or Bazett's formula may be used to correct the QT interval.
5. Oxygen saturation of ³ 90% on room air measured by pulse oximetry.
6. Adequate organ function as defined by the following laboratory values at Screening:
   a. Hemoglobin ≥9.0 g/dL without transfusion support within 2 weeks before Screening
   b. Platelet count ≥100,000/μL without transfusion support within 2 weeks before Screening
   c. Absolute neutrophil count >1000/mm³ (without granulocyte-colony stimulating factor support within 2 weeks before Screening)
   d. Creatinine clearance >45 mL/min as calculated using the CKD-EPI equation or creatinine <1.5×ULN
   e. ALT<2.5×ULN; ALT<5.0×ULN if known liver disease by cancer
   f. AST<2.5×ULN; AST<5.0×ULN if known liver disease by cancer
   g. Serum bilirubin <1.5×ULN (≤3.0×ULN if the participant has documented Gilbert syndrome)
   h. INR or prothrombin time (PT)≤1.5 ULN unless participant is receiving anticoagulant therapy and INR or PT is within expected or therapeutic range of intended use of anticoagulants
7. Willing and able to provide written informed consent.
8. Willing to perform and comply with all study procedures including undergoing study-related biopsies and attending clinic visits as scheduled.
9. Men must abstain from sperm donation during study treatment or for 4 months following last dose of study treatment.
10. Men and WOCBP must be willing to practice a highly effective method of contraception. Effective contraception options include:
   a. Double barrier (diaphragm, condom, or cervical cap with spermicidal foam, gel, or cream)
   b. Abstinence (must agree to use a double-barrier method if they become sexually active during the study)
   c. Implanted or intrauterine device with or without hormones in use for at least 6 consecutive months prior to study dosing and throughout the study duration
   d. Hormonal contraceptives (oral, injectable, implantable, transdermal, or vaginal hormonal device used consecutively for at least 3 consecutive months prior to the screening visit and throughout the study duration
   e. Male partner is surgically sterile (vasectomized) at least 6 months prior to the screening visit
   f. Same-sex partner

### Exclusion Criteria

Participants are excluded from the study if any of the following criteria are met:
1. Known active CNS metastasis and/or carcinomatous meningitis. Participants with previously treated brain metastases may participate provided they are radiologically stable, (e.g., without evidence of progression for at least 4 weeks by repeat imaging), clinically stable, and without requirement of steroid treatment for at least 14 days prior to the first dose of study intervention.
2. Pregnant or nursing women.
3. Must be ≥28 days beyond major surgery, including hepatectomy or joint replacement.
4. Prior allogeneic bone marrow transplantation or solid organ transplant.
5. Spinal cord compression not definitively treated with surgery and/or radiation.
6. Uncontrolled pleural effusion, pericardial effusion, or ascites requiring recurrent drainage procedures.
7. Any acute illness including fever (>100.4° F. or >38° C.) within 7 days prior to Day 1
8. Active systemic bacterial, fungal, or viral infection within 7 days prior to Day 1. Participant cannot have tested positive for COVID-19 within 7 days prior to Day 1.
9. Active infection with human immunodeficiency virus (HIV), hepatitis B virus (HBV), hepatitis C virus (HCV), as defined below:
   a. Positive serology for HIV-1
   b. Positive serology for HCV and has not received a documented curative therapy or quantitative RNA PCR is detectable
   c. Positive hepatitis B surface antigen or IgM, HBV core antibody unless quantitative DNA PCR is undetectable, and patient is stable on antiviral prophylaxis against HBV reactivation
10. Other primary malignancies, except:
   a. Adequately treated basal cell or squamous cell carcinoma
   b. In situ carcinoma of the cervix or bladder, treated curatively and without evidence of recurrence for at least 2 years prior to the study, or
   c. A primary malignancy which has been completely resected and in complete remission for at least 2 years
11. History of (noninfectious) pneumonitis/interstitial lung disease that required steroids or has current pneumonitis/interstitial lung disease.
12. Prior grade ≥3 immune-related AEs such as pneumonitis, colitis, hepatitis, nephritis; prior dermatitis and endocrinopathies are allowed provided corticosteroids are no longer required and endocrine-replacement therapy is stable and discontinued from prior therapy.
13. Active autoimmune disease not related to prior therapy for primary malignancy that has required systemic therapy in the last 1 year. Replacement therapy such as thyroxine, insulin, or physiologic steroid replacement therapy for adrenal or pituitary insufficiency are not considered systemic treatment. Celiac disease is allowed if adequately controlled by diet.
14. History of symptomatic congestive heart failure (New York Heart Association classes II-IV) or serious active arrhythmias or other clinically significant cardiac disease within 12 months of enrollment.
15. Toxicity from previous anti-cancer therapy defined as toxicities (other than alopecia, or laboratory values listed above) not yet resolved to NCI CTCAE v5.0 Grade ≤1 or baseline. Participants with chronic Grade 2 toxicities (eg, peripheral neuropathy, laboratory values) may be eligible per the discretion of the Investigator and Medical Monitor.
16. Has received:
   a. Radiotherapy within 2 weeks of first administration of anti-TROP2-CD89 CAR mRNA-LNP.
   b. Cytotoxic chemotherapy for treatment of the primary malignancy within 28 days or 5 half-lives, whichever is shorter, of administration of anti-TROP2-CD89 CAR mRNA-LNP.
   c. Immune therapy for primary malignancy (e.g, monoclonal antibody therapy, checkpoint inhibitors) within 28 days or 5 half-lives, whichever is shorter of first administration of anti-TROP2-CD89 CAR mRNA-LNP.
   d. Targeted therapies for primary malignancy within 28 days or 5 half-lives, whichever is shorter, of first administration of anti-TROP2-CD89 CAR mRNA-LNP.
   e. Anti-cancer vaccine within 12 weeks of first administration of anti-TROP2-CD89 CAR mRNA-LNP.
   f. COVID-19 mRNA vaccine within 6 weeks of first administration of anti-TROP2-CD89 CAR mRNA-LNP.
17. Has received a live vaccine ≤6 weeks prior to first administration of anti-TROP2-CD89 CAR mRNA-LNP.
18. Has received packed red blood cells or platelet transfusion within 2 weeks prior to first administration of anti-TROP2-CD89 CAR mRNA-LNP.
19. History of an allergic reaction to any of the excipients.
20. Enrollment in another interventional clinical trial within 28 days or 5 half-lives of the drug, whichever is shorter, of first administration of anti-TROP2-CD89 CAR mRNA-LNP.
21. Any other condition that, in the opinion of the Investigator, would make the participant unsuitable for the study or unable to comply with the study requirements.

TABLE 11A
Schedule of Events for Cohort 1, 2, and 3

| | | | | | Cycle 1 | | | | | | | |
|---|---|---|---|---|---|---|---|---|---|---|---|---|
| | Screening | | Dose | | | | Dose | | | | Dose | |
| | & Prep | | 1 ^{b} | | | | 2 | | | | 3 | |
| | Study Day | | | | | | | | | | | |
| | SI | Baseline^{e} | D 1 | D 2^{f} | D 3^{f} | D 8 | D 15 | D 16^{f} | D 17^{f} | D 22 | D 29 | D 30^{f} |
| | Windows (Days) | | | | | | | | | | | |
| | −28 | −5(+3) | 0 | 0 | 0 | ±1 | ±2 | 0 | 0 | ±1 | −2 | 0 |
| Informed | X | | | | | | | | | | | |
| Consent | | | | | | | | | | | | |
| Confirm | X | | | | | | | | | | | |
| Eligibility | | | | | | | | | | | | |
| Medical | X | | | | | | | | | | | |
| History | | | | | | | | | | | | |
| Complete | X | | | | | | | | | | | |
| Physical | | | | | | | | | | | | |
| Exam w/ | | | | | | | | | | | | |
| ICANS | | | | | | | | | | | | |
| Targeted | | | X | X | X | X | X | X | X | X | X | X |
| Physical | | | | | | | | | | | | |
| Exam | | | | | | | | | | | | |
| Vital Signs | X | | X | X | X | X | X | X | X | X | X | X |
| Weight | X | | X | | | | X | | | | X | |
| ECOG | X | | X | | | | X | | | | X | |
| Perf. Score | | | | | | | | | | | | |
| 12 Lead | X | | X ⁱ | | | | X ⁱⁱⁱ | | | | | |
| ECG | | | | | | | | | | | | |
| ECHO/MUGA ^{iv} | X | | | | | | | | | | | |
| MRI/CT | X^{v} | | | | | | | | | | | |
| Bone scan | X | | | | | | | | | | | |
| Chemistry | X | X^{v} | X | X | X | X | X | X | X | X | X | X |
| CA125/CEA/ | | | X | | | | X | | | | X | |
| CA19.9/ | | | | | | | | | | | | |
| CA15.3as | | | | | | | | | | | | |
| applicable | | | | | | | | | | | | |
| CBC w/ | X | X^{p} | X | X | X | X | X | X | X | X | X | X |
| diff. & | | | | | | | | | | | | |
| Platelets | | | | | | | | | | | | |
| Urinalysis | X | | Xs | X | | | X^{sError! Bookmark not defined.} | X | | | X^{s} | X |
| INR and | X | | X | X | | | X | X | | | X | X |
| aPTT | | | | | | | | | | | | |
| Fibrinogen | | | X | X | | | X | X | | | X | X |
| Lipid Panel | | | X | | | | | | | | X | |
| Complement | | | X | X | | | X | X | | | X | X |
| CPK | | | X | | | | X | | | | X | |
| HIV, HCV, | X | | | | | | | | | | | |
| HBV | | | | | | | | | | | | |
| Pregnancy | X | X | X | | | | X | | | | X | |
| Test | | | | | | | | | | | | |
| (WOCBP) | | | | | | | | | | | | |
| Serum | | X | X | X | | | X | X | | | X | X |
| Cytokines^{u} | | | | | | | | | | | | |
| CRP^{r} | | | X | | | | X | | | | X | |
| TSH, free | | | X | | | | | | | | | |
| T4, T3 | | | | | | | | | | | | |
| Ferritin^{u} | | | X | X | | | X | X | | | X | X |
| PK | | | X | X | X | X | X | | | | X | X |
| sampling | | | | | | | | | | | | |
| Anti-Drug | | | X | | | | X | | | | X | |
| Antibody | | | | | | | | | | | | |
| Correlative | | | X | X | | | | | | | X | X |
| Studies | | | | | | | | | | | | |
| CAR | | | X | X | | | | | | | X | X |
| Phenotyping^{xxi} | | | | | | | | | | | | |
| cfDNAxxii | | | X | | | | X | | | | X | |
| TCR | | | X | | | | | | | | X | |
| Sequencing | | | | | | | | | | | | |
| Tumor | X | | | | | | | | | | | |
| Biopsy | | | | | | | | | | | | |
| Anti- | | | X | | | | X | | | | X | |
| TROP2 DP | | | | | | | | | | | | |
| Dosing | | | | | | | | | | | | |
| AEs/IRs/ | X | X | X | X | X | X | X | X | X | X | X | X |
| AESI | | | | | | | | | | | | |
| Concomitant | X | X | X | X | X | X | X | X | X | X | X | X |
| Medication | | | | | | | | | | | | |
| (s) | | | | | | | | | | | | |
| Survival | | | | | | | | | | | | |
| Status | | | | | | | | | | | | |

| | | | | | | | | | Post Treatment | |
|---|---|---|---|---|---|---|---|---|---|---|
| | | | | | | End of | | | End | Long- |
| | | | MRI/ | | | Cycle 4 | Cycles 5-12^{a} | | of | term |
| | Cycle 1 | | CT | Doses 4-6 | | evaluation | Doses 7-14 | | Treatment^{c} | F/up^{d} |
| | Study Day | | | | | | | | | |
| | | | | | | | D 141, D 169 | D 142, D 170 | | |
| | | | | D 57 | D 58^{f} | | D 197, D 225 | D 198, D 226 | | |
| | | | | D 85, | Df86^{f} | | D 253, D 281 | D 254, D 282 | | Every 3 |
| | D 31^{f} | D 36 | D 50 | D 113^{g} | D 114^{f} | D 127 | D 309, D 337 | D 310, D 338 | D 351 | months |
| | Windows (Days) | | | | | | | | | |
| | 0 | ±1 | ±7 | ±4 | 0 | ±7 | ±4 | 0 | ±7 | 14 |
| Informed | | | | | | | | | | |
| Consent | | | | | | | | | | |
| Confirm | | | | | | | | | | |
| Eligibility | | | | | | | | | | |
| Medical | | | | | | | | | | |
| History | | | | | | | | | | |
| Complete | | | | | | | | | | |
| Physical | | | | | | | | | | |
| Exam w/ | | | | | | | | | | |
| ICANS | | | | | | | | | | |
| Targeted | X | X | X | X | X | X | X | X | X | |
| Physical | | | | | | | | | | |
| Exam | | | | | | | | | | |
| Vital Signs | X | X | X | X | X | X | X | X | X | |
| Weight | | | | X | | | X | | | |
| ECOG | | | | X | | | X | | X | |
| Perf. Score | | | | | | | | | | |
| 12 Lead | | | | | | | | | | |
| ECG | | | | | | | | | | |
| ECHO/MUGA ^{iv} | | | | | | | | | | |
| MRI/CT | | | X ^{vi} | | | X^{m} | X^{m} | | X | X^{d} |
| Bone scan | | | tbd | | | tbd | tbd | | tbd | tbd |
| Chemistry | X | X | | X | X | X | X | X | X | |
| CA125/CEA/ | | | X | | | X | | | X | X |
| CA19.9/ | | | | | | | | | | |
| CA15.3as | | | | | | | | | | |
| applicable | | | | | | | | | | |
| CBC w/ | X | X | | X | X | X | X | X | X | |
| diff. & | | | | | | | | | | |
| Platelets | | | | | | | | | | |
| Urinalysis | | | | Xs | | X | Xs | | X | |
| INR and | | | | X | X | X | X | X | X | |
| aPTT | | | | | | | | | | |
| Fibrinogen | | | | X | X | X | X | X | X | |
| Lipid Panel | | | | | | | | | | |
| Complement | | | | X | X | | X | X | | |
| CPK | | | | X | | X | X | X | X | |
| HIV, HCV, | | | | | | | | | | |
| HBV | | | | | | | | | | |
| Pregnancy | | | | X | | | X | | | |
| Test | | | | | | | | | | |
| (WOCBP) | | | | | | | | | | |
| Serum | | | | X | X | | X | X | | |
| Cytokines^{u} | | | | | | | | | | |
| CRP^{r} | | | | | | | | | | |
| TSH, free | | | | | | X | | | X | |
| T4, T3 | | | | | | | | | | |
| Ferritin^{u} | | | | X | X | | X | X | | |
| PK | X | X | | X | | | X | | | |
| sampling | | | | | | | | | | |
| Anti-Drug | | | | X | | X | X | | X | |
| Antibody | | | | | | | | | | |
| Correlative | | | | | | X | | | X | |
| Studies | | | | | | | | | | |
| CAR | | | | | | X | | | X | |
| Phenotyping^{xxi} | | | | | | | | | | |
| cfDNAxxii | | | X | | | X | | | X | X |
| TCR | | | | | | X | | | X | |
| Sequencing | | | | | | | | | | |
| Tumor | X | | | | | X | | | X | |
| Biopsy | | | | | | | | | | |
| Anti- | | | | X | | | X | | | |
| TROP2 DP | | | | | | | | | | |
| Dosing | | | | | | | | | | |
| AEs/IRs/ | X | X | | X | X | X | X | X | X | |
| AESI | | | | | | | | | | |
| Concomitant | X | X | | X | X | X | X | X | X | |
| Medication | | | | | | | | | | |
| (s) | | | | | | | | | | |
| Survival | | | | | | X | | | X | X |
| Status | | | | | | | | | | |

AB = antibody; AE = adverse event; AESI = adverse event of special interest; AR = adverse reaction; BP = blood pressure; CBC = complete blood count; CPK = creatine phosphokinase; CRP = C-reactive protein; CT = computed tomography; D = day; Diff = differential; ECG = electrocardiogram; ECOG = eastern cooperative oncology group; ECHO = echocardiogram; Hep BSAg = Hepatitis B Surface Antigen; HIV = human immunodeficiency virus; HR = heart rate; ICANS = immune effector cell-associated neurotoxicity syndrome; INR = international normalized ratio; IR = infusion reaction; PD = progressive disease; Perf. = performance; PK = pharmacokinetics; PTT = partial thromboplastin time; RR = respiratory rate; S = screening; T3 = triiodothyronine; T4 = thyroxine; tbd = to be determined; TCR = T-cell receptor; TSH = thyroid stimulating hormone; WOCBP = women of childbearing potential.
Related to the Table 11A above,
^{a}Patients with SD or a PR will continue Cycles 5-12 until PD or CR. Dosing days are D141, D169, D197, D225, D253, D281, D309, D337 with a window of ± 4 days. Follow-up visits must occur on the day following dosing; planned visit days are D142, D170, D198, D226, D254, D282, D310, and D338. End of Treatment evaluation on D351 ± 7 days.
^{b} Patients will be admitted for 24-hour observation for Dose 1.
^{c}EOT evaluation will be performed 14 days after last treatment or in event of premature study termination.
^{d}Follow-up, including MRI/CT, whole blood for cfDNA, and Survival Status, required every 3 months (±14 days) for up to 2 years to patients who have not progressed or have started a new line of therapy, or only until resolution of any treatment-related abnormalities once patient progresses. In patients with PD or who have started a new line of therapy, only Survival Status will be required until Study is complete.
^{e}If Screening visit is <14 days prior to C1D1, Baseline visit is optional. If Screening visit is >14 days prior to C1D1, Baseline visit is required. If Baseline labs are obtained, they do not need to be repeated on C1D1.
^{f}If Dosing is delayed, subsequent visits are also delayed. For Cycle 1, follow-up visits must be one and two days after dosing. For Cycles 2-4, follow-up visits are the day immediately following dosing.
^{g}Dosing occurs every 28 days (D57, D85, D113).
^{h} BP, HR, Temperature, RR, and pulse oximetry to be obtained pre-dose (−15 min), 15 ± 5 min, 30 ± 5 min, 1, 2, 4, and 6 hours (±10 min) post start of infusion for Cycle 1. BP, HR, Temperature and pulse oximetry to be obtained pre-dose (−15 min), 15 ± 5 min, 30 ± 5 min, 1, 2 hours (±10 min) post start of infusion for Cycles 2-12. Height to be taken at Screening.
ⁱ Weight will be measured pre-dose (dosing day up to −8 days).
^{j} ECG prior to Dose 1 and Dose 2 may be obtained up to 3 days prior to dosing visit.
^{k} ECHO/MUGA may be repeated during the study as clinically indicated.
^{l} MRI/CT performed following the subject's last line of therapy and prior to signing consent and within a 6 month period may be used for eligibility. However, another MRI/CT may be performed within the screening window.
^{m}MRI/CT should be performed D50 ± 7 days, D127 ± 7 days and then every 12 weeks ± 14 days until the end of the study. Additionally, MRI/CT required 4 weeks after first occurrence of any PR and CR to confirm the response.
ⁿ A bone scan is required at Screening (prior to the MRI/CT) in Participants with previously documented or suspected bone metastases and in Participants with advanced breast cancer.
^{o} Serum chemistry to be completed locally (prior to dosing [up to −3 days] on dosing days). Serum chemistry to include sodium, potassium, magnesium, phosphorus, chloride, bicarbonate, urea, creatinine, glucose, calcium, total protein, albumin, total bilirubin, AST (SGOT), ALT (SGPT), GGT, alkaline phosphatase.
^{p}Labs not within the screening criteria are to be reported to the Medical Monitor for review of continued eligibility for the study.
^{q} See procedures for assessing tumor biomarkers. If baseline level is within normal range on C1D1, no further testing is required. If baseline level is outside the normal range, collect samples as noted above on dosing days (−3 days) and within ±7 days of imaging. All tests are performed locally.
^{r}CBC with differential, platelets, and CRP to be obtained pre-dose and anytime a subject experiences a fever ≥ Grade 2 (39.0° C. or 102.3° F.) or if patient has signs and/or symptoms consistent with CRS or ICANS. Hematology laboratory assessments may be performed up to 3 days prior to dosing.
^{s}When urinalysis is scheduled on a dosing day, the assessment may be performed up to 3 days prior to dosing.
^{t} INR, aPTT and Fibrinogen required pre-dose (dosing day up to −3 days) and 24 hours (±2 hr) post-dose; and anytime a subject experiences a fever ≥ Grade 2 (39.0° C. or 102.3° F.) or if patient has signs and/or symptoms consistent with CRS or ICANS.
^{u}Complement, ferritin, and serum cytokines will be assessed a) pre-dose, b) 6 hours (±10 min) after dosing and c) 24 hours (±2 hr) after dosing. All three plus IL-6 (assessed locally) will also be assessed anytime a patient experiences a fever ≥ Grade 2 (39.0° C. or 102.3° F.) or if the patient has signs and/or symptoms consistent with CRS or ICANS. IL-6 (assessed locally) required at baseline visit (There is potential for future centralized testing; a larger cytokine panel may be included at the discretion of the Investigator). Ferritin samples scheduled to be collected predose on dosing days may be collected up to 3 days prior to dosing.
^{v} CPK samples scheduled to be collected predose on dosing days may be collected up to 3 days prior to dosing.
^{w} Viral serology tests include HCV antibody, HBsAg (HBV surface antigen) and HBV core antibody (see Exclusion Criterion).
^{x}For WOCBP a serum pregnancy test must be done at screening. A urine pregnancy test may be performed at other visits (−3 day window). Women age < 60 years and amenorrheic for 12 months or more, collect FSH and estradiol at Screening to ensure values are in the postmenopausal range.
^{y} See PK sampling times and procedures.
^{z} Presence of Anti-Drug Antibody should be assessed prior to each dose; EOT.
^{aa} Whole blood for correlative studies to be taken pre-dose and 24-hrs (±2 hrs) post dose.
^{bb} Whole blood for CAR phenotyping to be taken pre-dose, 6-hr (±20 min) and 24-hr (±2 hrs) post dose.
^{cc} Whole blood for cfDNA sample to be taken before each of the 3 doses in Cycle 1 (ie, pre-dose C1D1, C1D2, C1D3) and any time an MRI/CT is performed (±7 days).
^{dd} Whole blood for TCR sequencing to be done pre-dose only.

TABLE 11B
Schedule of Events for Cohort 4

| | | | Cycle 1 | | | | | | | | | | |
|---|---|---|---|---|---|---|---|---|---|---|---|---|---|
| | Screening | | Dose | | | Dose | | | Dose | | | | DLT |
| | & Prep | | 1 | | | 2 | | | 3 | | | | Evaluation |
| | Study Day | | | | | | | | | | | | |
| | | Base | | | | | | | | | | | |
| | S1 | Line | D 1 | D 2 | D 3^{Error! Bookmark not defined.} | D 8 | D 9 | 10 | D 15 | 16 | D 17 | D 22 | D 30 |
| | Window (Days) | | | | | | | | | | | | |
| | | −5 | | | | | | | | | | | |
| | −28 | (+3) | 0 | 0 | 0 | ±2 | 0 | 0 | ±2 | 0 | 0 | ±1 | +2 |
| Informed | X | | | | | | | | | | | | |
| Consent | | | | | | | | | | | | | |
| Confirm | X | | | | | | | | | | | | |
| Eligibility | | | | | | | | | | | | | |
| Medical | X | | | | | | | | | | | | |
| History | | | | | | | | | | | | | |
| Complete | X | | | | | | | | | | | | |
| Physical | | | | | | | | | | | | | |
| Exam w/ | | | | | | | | | | | | | |
| ICANS | | | | | | | | | | | | | |
| Targeted | | | X | X | X | X | X | X | X | X | X | | X |
| Physical | | | | | | | | | | | | | |
| Exam | | | | | | | | | | | | | |
| Vital Signs | X | | X | X | X | X | X | X | X | X | X | | X |
| Weight | X | | X | | | X | | | X | | | | |
| ECOG | X | | X | | | X | | | X | | | | |
| Perf. Score | | | | | | | | | | | | | |
| 12 Lead | X | | X | | | X | | | | | | | |
| ECG | | | | | | | | | | | | | |
| ECHO/MUGA | X | | | | | | | | | | | | |
| MRI/CT | X | | | | | | | | | | | | |
| Bone scan | X | | | | | | | | | | | | |
| Chemistryⁱ | X | X | X | X | X | X | X | X | X | X | X | | X |
| CA125/CEA/ | | | X | | | X | | | X | | | | |
| CA19.9/ | | | | | | | | | | | | | |
| CA15.3 as | | | | | | | | | | | | | |
| applicable | | | | | | | | | | | | | |
| CBC w/ | X | X | X | X | X | X | X | X | X | X | X | | X |
| diff. & | | | | | | | | | | | | | |
| Platelets | | | | | | | | | | | | | |
| Urinalysis | X | | X | X | | X | X | | X | X | | | X |
| INR and | X | | X | X | | X | X | | X | X | | | |
| aPTT | | | | | | | | | | | | | |
| Fibrinogen | | | X | X | | X | X | | X | X | | | |
| Lipid Panel | | | X | | | | | | X | | | | |
| Complement | | | X | X | | X | X | | X | X | | | |
| CPK | | | X | | | X | | | X | | | | |
| HIV, HCV, | X | | | | | | | | | | | | |
| HBV | | | | | | | | | | | | | |
| Pregnancy | X | X | X | | | X | | | X | | | | |
| Test | | | | | | | | | | | | | |
| (WOCBP) | | | | | | | | | | | | | |
| Serum | | X | X | X | | X | X | | X | X | | | |
| Cytokines^{Error! Bookmark not defined.} | | | | | | | | | | | | | |
| CRP | | | X | | | X | | | X | | | | |
| TSH, free | | | X | | | | | | | | | | |
| T4, T3 | | | | | | | | | | | | | |
| Ferritin | | | X | X | | X | X | | X | X | | | |
| PK | | | X | X | X | X | | | X | X | X | X | |
| sampling | | | | | | | | | | | | | |
| Anti-Drug | | | X | | | X | | | X | | | | |
| Antibody | | | | | | | | | | | | | |
| Correlative | | | X | X | | | | | X | X | | | |
| Studies | | | | | | | | | | | | | |
| CAR | | | X | X | | | | | X | X | | | |
| Phenotyping | | | | | | | | | | | | | |
| cfDNA | | | X | | | X | | | X | | | | |
| TCR | | | X | | | | | | X | | | | |
| Sequencing | | | | | | | | | | | | | |
| Tumor | X | | | | | | | | | | X | | |
| Biopsy | | | | | | | | | | | | | |
| DP 302 | | | X | | | X | | | X | | | | |
| Dosing | | | | | | | | | | | | | |
| AEs/IRs/ | X | X | X | X | X | X | X | X | X | X | X | X | X |
| AESI | | | | | | | | | | | | | |
| Concomitant | X | X | X | X | X | X | X | X | X | X | X | X | X |
| Medication | | | | | | | | | | | | | |
| (s) | | | | | | | | | | | | | |
| Survival | | | | | | | | | | | | | |
| Status | | | | | | | | | | | | | |

Cycles 2-4

| End | |
|---|---|
| of | Post Treatment |

| | | | | Cycle | | | End | |
|---|---|---|---|---|---|---|---|---|
| | MRI/ | | | 4 | Cycles 5-12 | | of | Long-term |
| | CT | Doses 4-6 | | Evaluation | Doses 7-14 | | Treatment | F/up |
| | Study Day | | | | | | | |
| | | | | | D 127, D 155, | D 128, D 156, | | |
| | | D 43 | D 44, | | D 183, D 211, | D 184, D 212, | | |
| | | D 71 | D 72, | | D 239, D 267, | D 240, D 268, | | Every 3 |
| | D 50 | D 99 | D 100^{Error! Bookmark not defined.} | D 113 | D 295, D 323 | D 296, D 324 | D 337 | months |
| | Window (Days) | | | | | | | |
| | ±7 | ±4 | 0 | ±7 | ±4 | 0 | ±7 | ±14 |
| Informed | | | | | | | | |
| Consent | | | | | | | | |
| Confirm | | | | | | | | |
| Eligibility | | | | | | | | |
| Medical | | | | | | | | |
| History | | | | | | | | |
| Complete | | | | | | | | |
| Physical | | | | | | | | |
| Exam w/ | | | | | | | | |
| ICANS | | | | | | | | |
| Targeted | X | X | X | X | X | X | X | |
| Physical | | | | | | | | |
| Exam | | | | | | | | |
| Vital Signs | X | X | X | X | X | X | X | |
| Weight | | X | | | X | | | |
| ECOG | | X | | | X | | X | |
| Perf. Score | | | | | | | | |
| 12 Lead | | | | | | | | |
| ECG | | | | | | | | |
| ECHO/MUGA | | | | | | | | |
| MRI/CT | X | | | X | X | | X | X |
| Bone scan | tbd | | | tbd | tbd | | tbd | tbd |
| Chemistryⁱ | | X | X | X | X | X | X | |
| CA125/CEA/ | X | | | X | | | X | X |
| CA19.9/ | | | | | | | | |
| CA15.3 as | | | | | | | | |
| applicable | | | | | | | | |
| CBC w/ | | X | X | X | X | X | X | |
| diff. & | | | | | | | | |
| Platelets | | | | | | | | |
| Urinalysis | | X | | X | X | | X | |
| INR and | | X | X | X | X | X | X | |
| aPTT | | | | | | | | |
| Fibrinogen | | X | X | X | X | X | X | |
| Lipid Panel | | | | | | | | |
| Complement | | X | X | | X | X | | |
| CPK | | X | | X | X | X | X | |
| HIV, HCV, | | | | | | | | |
| HBV | | | | | | | | |
| Pregnancy | | X | | | X | | | |
| Test | | | | | | | | |
| (WOCBP) | | | | | | | | |
| Serum | | X | X | | X | X | | |
| Cytokines^{Error! Bookmark not defined.} | | | | | | | | |
| CRP | | | | | | | | |
| TSH, free | | | | X | | | X | |
| T4, T3 | | | | | | | | |
| Ferritin | | X | X | | X | X | | |
| PK | | X | | | X | | | |
| sampling | | | | | | | | |
| Anti-Drug | | X | | X | X | | X | |
| Antibody | | | | | | | | |
| Correlative | | | | X | | | X | |
| Studies | | | | | | | | |
| CAR | | | | X | | | | |
| Phenotyping | | | | | | | | |
| cfDNA | X | | | X | | | X | X |
| TCR | | | | X | | | X | |
| Sequencing | | | | | | | | |
| Tumor | | | | X | | | X | |
| Biopsy | | | | | | | | |
| DP 302 | | X | | | X | | | |
| Dosing | | | | | | | | |
| AEs/IRs/ | | X | X | X | X | X | X | |
| AESI | | | | | | | | |
| Concomitant | | X | X | X | X | X | X | |
| Medication | | | | | | | | |
| (s) | | | | | | | | |
| Survival | | | | X | | | X | X |
| Status | | | | | | | | |

AB = antibody; AE = adverse event; AESI = adverse event of special interest; AR = adverse reaction; BP = blood pressure; CBC = complete blood count; CPK = creatine phosphokinase; CRP = C-reactive protein; CT = computed tomography; D = day; Diff = differential; DLT = dose limiting toxicity; ECG = electrocardiogram; ECOG = eastern cooperative oncology group; ECHO = echocardiogram; Eval. = evaluation; Antigen; HIV = human immunodeficiency virus; HR = heart rate; ICANS = immune effector cell-associated neurotoxicity syndrome; INR = international normalized ratio; IR = infusion reaction; PD = progressive disease; Perf. = performance; PK = pharmacokinetics; PTT = partial thromboplastin time; RR = respiratory rate; S = screening; T3 = triiodothyronine; T4 = thyroxine; tbd = to be determined; TCR = T-cell receptor; TSH = thyroid stimulating hormone; WOCBP = women of childbearing potential
Related to the Table 11B above,
^{a} Patients with SD or a PR will continue Cycles 5-12 until PD or CR. Dosing days are: D127, D155, D183, D211, D239, D267, D295, D323 with a window of ± 4 days. Follow-up visits must occur on the day following dosing; planned visit days are D128, D156, D184, D212, D240, D268, D296, and D246. EOT evaluations will be completed on D338 ± 7 days.
^{b} Patients will be admitted for 24-hour observation for Dose 1.
^{c} EOT evaluation will be performed 14 days after last treatment or in event of premature study termination as described.
^{d} Follow-up, including MRI/CT, whole blood for cfDNA, and Survival Status, required every 3 months (±14 days) for up to 2 years for patients who have not progressed or have started a new line of therapy, or only until resolution of any treatment-related abnormalities once patient progresses. In patients with PD or who have started a new line of therapy, only Survival Status will be required until Study is complete.
^{e} If Screening visit is <14 days prior to C1D1, Baseline visit is optional. If Screening visit is >14 days prior to C1D1, Baseline visit is required. If Baseline labs are obtained, they do not need to be repeated on C1D1.
^{f} If Dosing is delayed, subsequent visits are also delayed. For Cycle 1, follow-up visits must be one and two days after dosing. For Cycles 2-4, follow-up visits are the day immediately following dosing.
^{g} Dosing occurs every 28 days (D57, D85, D113).
^{h} BP, HR, Temperature, RR, and pulse oximetry to be obtained pre-dose (−15 min), 15 ± 5 min, 30 ± 5 min, 1, 2, 4, and 6 hours (±10 min) post start of infusion for Cycle 1. BP, HR, temperature and pulse oximetry to be obtained pre-dose (−15 min), 15 ± 5 min, 30 ± 5 min, 1, 2 hours (±10 min) post start of infusion for Cycles 2-12. Height to be taken at Screening.
ⁱWeight will be measured pre-dose (dosing day up to −8 days).
^{j} ECG prior to Dose 1 and Dose 2 may be obtained up to 3 days prior to dosing visit.
^{k} ECHO/MUGA may be repeated during the study as clinically indicated.
^{l} MRI/CT performed following the subject's last line of therapy and prior to signing consent and within a 6 month period may be used for eligibility. However, another MRI/CT may be performed within the screening window.
^{m} MRI/CT should be performed D50 ± 7 days, Day 113 ± 7 days and then every 12 weeks ± 14 days until the end of the study. Additionally, MRI/CT required 4 weeks after first occurrence of any PR and CR to confirm response.
ⁿ A bone scan is required at Screening in Participants with previously documented or suspected bone metastases and in Participants with advanced breast cancer.
^{o} Serum chemistry to be completed locally (prior to dosing [up to −3 days] on dosing days). Seru chemistry to include sodium, potassium, magnesium, phosphorus, chloride, bicarbonate, urea, creatinine, glucose, calcium, total protein, albumin, total bilirubin, AST (SGOT), ALT (SGPT), GGT, alkaline phosphatase.
^{p} Labs not within the screening criteria are to be reported to the Medical Monitor for review of continued eligibility for the study.
^{q} If baseline level is within normal range on C1D1, no further testing is required. If baseline level is outside the normal range, collect samples as noted above on dosing days (−3 days) and within ±7 days of imaging. All tests are performed locally.
^{r} CBC with differential, platelets, and CRP to be obtained pre-dose and anytime a subject experiences a fever ≥ Grade 2 (39.0° C. or 102.3° F.) or if patient has signs and/or symptoms consistent with CRS or ICANS. Hematology laboratory assessments may be performed up to 3 days prior to dosing.
^{s} When urinalysis is scheduled on a dosing day, the assessment may be performed up to 3 days prior to dosing.
^{t} INR, aPTT and Fibrinogen required pre-dose (dosing day up to −3 days) and 24 hours (±2 hr) post-dose; and anytime a subject experiences a fever ≥ Grade 2 (39.0° C. or 102.3° F.) or if patient has signs and/or symptoms consistent with CRS or ICANS.
^{u} Complement, ferritin, and serum cytokines will be assessed a) pre-dose, b) 6 hours (±10 min) after dosing and c) 24 hours (±2 hr) after dosing. All three plus IL-6 (assessed locally) will also be assessed anytime a patient experiences a fever ≥ Grade 2 (39.0° C. or 102.3° F.) or if the patient has signs and/or symptoms consistent with CRS or ICANS. IL-6 (assessed locally) required at baseline visit (There is potential for future centralized testing; a larger cytokine panel may be included at the discretion of the Investigator). Ferritin samples scheduled to be collected predose on dosing days may be collected up to 3 days prior to dosing.
^{v} CPK samples scheduled to be collected predose on dosing days may be collected up to 3 days prior to dosing.
^{w} Viral serology tests include HCV antibody, HBsAg (HBV surface antigen) and HBV core antibody.
^{x} For WOCBP a serum pregnancy test must be done at screening. A urine pregnancy test may be performed at other visits (−3 day window). Women age < 60 years and amenorrheic for 12 months or more, collect FSH and estradiol at Screening to ensure values are in the postmenopausal range.
^{y} PK sampling times and procedures.
^{z} Presence of Anti-Drug Antibody should be assessed prior to each dose; EOT.
^{aa} Whole blood for correlative studies to be taken pre-dose and 24-hrs (±2 hrs) post dose.
^{bb} Whole blood for CAR phenotyping to be taken pre-dose, 6-hr (±20 min) and 24-hr (±2 hrs) post dose.
^{cc} Whole blood for cfDNA sample to be taken before each of the 3 doses in Cycle 1 (i.e, pre-dose C1D1,
C1D2, C1D3) and any time an MRI/CT is performed (±7 days).
^{dd} Whole blood for TCR sequencing to be done pre-dose only.

TABLE 11C
Schedule of Events for Cohorts 5, 6 and 7

| | | | Cycle 1 & 2^{a} | | | | | | | |
|---|---|---|---|---|---|---|---|---|---|---|
| | Screening | | Dose | | | | Dose | | | |
| | & Prep | | 1 | | | | 2 | | | |
| | Study Day | | | | | | | | | |
| | | | | | | | | | | D 22 |
| | | | | | | | | | D 17g | (Cycle |
| | | Base | | | | | | | (Cycle 1 | 1 |
| | S1 | Line^{f} | D 1 | D 2g | D 3g | D 8 | D 15 | D 16g | only) | only) |
| | Window (Days) | | | | | | | | | |
| | | −5 | | | | | | | | |
| | −28 | (+4) | 0 | 0 | 0 | ±1 | ±2 | 0 | 0 | ±1 |
| Informed Consent | X | | | | | | | | | |
| Confirm Eligibility | X | | | | | | | | | |
| Medical History | X | | | | | | | | | |
| Complete Physical Exam w/ | X | | | | | | | | | |
| ICANS | | | | | | | | | | |
| Targeted Physical Exam | | | X | X | X | X | X | X | X | X |
| Vital Signs^{b} | X | | X | X | X | X | X | X | X | X |
| Weight^{c} | X | | X | | | | X | | | |
| ECOG Perf. Score | X | | X | | | | X | | | |
| 12 Lead ECG | X | | X^{d} | | | | X | | | |
| ECHO/MUGA | X | | | | | | | | | |
| MRI/CT | Xm | | | | | | | | | |
| Bone scan | Xo | | | | | | | | | |
| Chemistry^{p} | X | Xq | X | X | X | X | X | X | X | X |
| CA125/CEA/ | | | X | | | | X | | | |
| CA19.9/CA15.3 as | | | | | | | | | | |
| applicable | | | | | | | | | | |
| CBC w/diff. & Platelets | X | X | X | X | X | X | X | X | X | X |
| Urinalysis | X | | X | X | | | X | X | | |
| INR and aPTT | X | | X | X | | | X | X | | |
| Fibrinogen | | | X | X | | | X | X | | |
| Lipid Panel | | | X | | | | | | | |
| Complement | | | X | X | | | X | X | | |
| CPK | | | X | | | | X | | | |
| HIV, HCV, HBV | X | | | | | | | | | |
| Pregnancy Test (WOCBP) | X | X | X | | | | X | | | |
| Serum Cytokine | | X | X | X | | | X | X | | |
| CRP | | | X | | | | X | | | |
| TSH, free T4, T3 | | | X | | | | | | | |
| Ferritin | | | X | X | | | X | X | | |
| PK sampling | | | X | X | X | X | X | | | |
| Anti-Drug Antibody | | | X | | | | X | | | |
| Correlative Studies | | | X | X | | | | | | |
| CAR Phenotyping | | | X | X | | | | | | |
| cfDNA | X | | | | | | X | | | |
| TCR Sequencing | | | X | | | | | | | |
| Tumor Biopsy | X | | | | X^{ee} | | | | | |
| DP Dosing | | | X | | | | X | | | |
| AEs/IRs/AESI/Con. Meds | X | X | X | X | X | X | X | X | X | X |
| Survival Status | | | | | | | | | | |

| | | Cycles 3-4 | | | | | Post Treatment | |
|---|---|---|---|---|---|---|---|---|
| | | | | End of | Cycles 5-12a | | End | |
| | MRI/ | Doses 5-8 | | Cycle 4 | Doses 9+ | | of | Long-term |
| | CT | (Q14D) | | Evaluation | (Q14D) | | Treatment^{d} | F/up^{a} |
| | Study Day | | | | | | | |
| | | | 1- | | | 1- | | |
| | | | day | | | day | | |
| | | Dosing | post | | Dosing | post | | Every 3 |
| | D 50 | Day^{h} | dose^{h} | D 106 | Day | dose | D 337 | months |
| | Window (Days) | | | | | | | |
| | ±7 | ±4 | 0 | ±7 | ±4 | 0 | ±7 | ±14 |
| Informed Consent | | | | | | | | |
| Confirm Eligibility | | | | | | | | |
| Medical History | | | | | | | | |
| Complete Physical Exam w/ | | | | | | | | |
| ICANS | | | | | | | | |
| Targeted Physical Exam | X | X | X | X | X | X | X | |
| Vital Signs^{b} | X | X | X | X | X | X | X | |
| Weight^{c} | | X | | | X | | | |
| ECOG Perf. Score | | X | | | X | | X | |
| 12 Lead ECG | | | | | | | | |
| ECHO/MUGA | | | | | | | | |
| MRI/CT | Xn | | | Xⁿ | Xⁿ | | X | Xe |
| Bone scan | tbd | | | tbd | tbd | | tbd | tbd |
| Chemistry^{p} | | X | X | X | X | X | X | |
| CA125/CEA/ | X | | | X | | | X | X |
| CA19.9/CA15.3 as | | | | | | | | |
| applicable | | | | | | | | |
| CBC w/diff. & Platelets | | X | X | X | X | X | X | |
| Urinalysis | | X | | X | X | | X | |
| INR and aPTT | | X | X | X | X | X | X | |
| Fibrinogen | | X | X | X | X | X | X | |
| Lipid Panel | | | | | | | | |
| Complement | | X | X | | X | X | | |
| CPK | | X | | X | X | X | X | |
| HIV, HCV, HBV | | | | | | | | |
| Pregnancy Test (WOCBP) | | X | | | X | | | |
| Serum Cytokine | | X | X | | X | X | | |
| CRP | | | | | | | | |
| TSH, free T4, T3 | | | | X | | | X | |
| Ferritin | | X | X | | X | X | | |
| PK sampling | | X | | | X | | | |
| Anti-Drug Antibody | | X | | X | X | | X | |
| Correlative Studies | | | | X | | | X | |
| CAR Phenotyping | | | | X | | | X | |
| cfDNA | X | | | X | | | X | X |
| TCR Sequencing | | | | X | | | X | |
| Tumor Biopsy | | | | X | | | X | |
| DP Dosing | | X | | | X | | | |
| AEs/IRs/AESI/Con. Meds | | X | X | X | X | X | X | |
| Survival Status | | | | X | | | X | X^{e} |

AB = antibody; AE = adverse event; AESI = adverse event of special interest; BP = blood pressure; CBC = complete blood count; Con. Meds = concomitant medications; CPK = creatine phosphokinase; CRP = C-reactive protein; CT = computed tomography; D = day; Diff = differential; ECG = electrocardiogram; ECOG = eastern cooperative oncology group; ECHO = echocardiogram; Hep BSAg = Hepatitis B Surface Antigen; HIV = human immunodeficiency virus; HR = heart rate; ICANS = immune effector cell-associated neurotoxicity syndrome; INR = international normalized ratio; IR = infusion reaction; PD = progressive disease; Perf. = performance; PK = pharmacokinetics; PTT = partial thromboplastin time; RR = respiratory rate; S = screening; T3 = triiodothyronine; T4 = thyroxine; tbd = to be determined; TCR = T-cell receptor; TSH = thyroid stimulating hormone; WOCBP = women of childbearing potential.
Related to the Table 11C above,
^{a}Dose 1 Cycle 2 occurs approximately on D29 ± 2 days with follow-up visits on D30, D31, D36. Dose 2 for Cycle 2 occurs approximately on D43 ± 2 days with follow-up visit on D44
^{b}Patients with SD or a PR will continue Cycles 5-12 until PD or CR. Dosing days are Q14D with a window of ± 4 days. Follow-up visits must occur on the day following dosing. Dosing days are
D113, D127, D141, D155, D169, D183, D197, D211, D225, D239, D253, D267, D281, D295, D309, D323. Follow-up visits occur on D114, D128, D142, D156, D170, D184, D198, D212, D226, D240, D254, D268, D282, D296, D310, D324. End of Treatment evaluation on D337 ± 7 days.
^{c}Patients will be admitted for 24-hour observation for Dose 1.
^{d}EOT evaluation will be performed 14 days after last treatment or in event of premature study termination.
^{e}Follow-up, including MRI/CT, whole blood for cfDNA, and Survival Status, required every 3 months (±14 days) for up to 2 years for patients who have not progressed or have started a new line of therapy, or only until resolution of any treatment-related abnormalities once patient progresses. In
patients with PD or who have started a new line of therapy, only Survival Status will be required until Study is complete.
^{f}If Screening visit is ≤14 days prior to C1D1, Baseline visit is optional. If Screening visit is >14 days prior to C1D1, Baseline visit is required. If Baseline labs are obtained, they do not need to be repeated on C1D1.
^{g} If Dosing is delayed, subsequent visits are also delayed. For Cycle 1 & 2, follow-up visits must be one and two days after dosing. For Cycles 3+, follow-up visits are the day immediately following dosing.
^{h}Dosing occurs every 14 days and follow-up visits are the day immediately following dosing. Dosing days are D57, D71, D85, D99. Follow-up visits occur on D58, D72, D86, D100.
ⁱ BP, HR, Temperature, RR, and pulse oximetry to be obtained pre-dose (−15 min), 15 ± 5 min, 30 ± 5 min, 1, 2, 4, and 6 hours (±10 min) post start of infusion for Cycle 1. BP, HR, Temperature and pulse oximetry to be obtained pre-dose (−15 min), 15 ± 5 min, 30 ± 5 min, 1, 2 hours (±10 min) post start of infusion for Cycles 2-12. Height to be taken at Screening.
^{j} Weight will be measured pre-dose (dosing day up to −15 days).
^{k} Cycle 1 only. ECG prior to Dose 1 and Dose 2 may be obtained up to 3 days prior to dosing visit.
^{l} ECHO/MUGA may be repeated during the study as clinically indicated.
^{m} MRI/CT performed following the subject's last line of therapy, prior to signing consent, and within a 6 month period may be used for eligibility. However, another MRI/CT may be performed within the screening window.
ⁿMRI/CT should be performed D50 ± 7 days, D106 ± 7 days and then every 12 weeks ± 14 days until the end of the study. Additionally, MRI/CT required 4 weeks after first occurrence of any PR and CR to confirm the response.
^{o} A bone scan is required at Screening (prior to the MRI/CT) in Participants with previously documented or suspected bone metastases and in Participants with advanced breast cancer.
^{p}Serum chemistry to be completed locally (prior to dosing [up to −3 days] on dosing days). Serum chemistry to include sodium, potassium, magnesium, phosphorus, chloride, bicarbonate, urea, creatinine, glucose, calcium, total protein, albumin, total bilirubin, AST (SGOT), ALT (SGPT), GGT, alkaline phosphatase.
^{q} Results of laboratory assessments that are not within the screening criteria are to be reported to the Medical Monitor for review of continued eligibility for the study.
^{r} If baseline level is within normal range on C1D1, no further testing is required. If baseline level is outside the normal range, collect samples as noted above on dosing days (−3 days) and within ± 7 days of imaging. All tests are performed locally.
^{s} CBC with differential, platelets, and CRP to be obtained pre-dose and anytime a subject experiences a fever ≥ Grade 2 (39.0° C. or 102.3° F.) or if patient has signs and/or symptoms consistent with CRS or ICANS. Hematology laboratory assessments may be performed up to 3 days prior to dosing.
^{t} When urinalysis is scheduled on a dosing day, the assessment may be performed up to 3 days prior to dosing.
^{u} INR, aPTT and Fibrinogen required pre-dose (dosing day up to −3 days) and 24 hours (±2 hr) post-dose; and anytime a subject experiences a fever ≥ Grade 2 (39.0° C. or 102.3° F.) or if patient has signs and/or symptoms consistent with CRS or ICANS.
^{v} Complement, ferritin, and serum cytokines will be assessed a) pre-dose, b) 6 hours (±10 min) after dosing and c) 24 hours (±2 hr) after dosing. All three plus IL-6 (assessed locally) will also be assessed anytime a patient experiences a fever ≥ Grade 2 (39.0° C. or 102.3° F.) or if the patient has signs and/or symptoms consistent with CRS or ICANS. IL-6 (assessed locally) required at baseline visit (There is potential for future centralized testing; a larger cytokine panel may be included at the discretion of the Investigator). Ferritin samples scheduled to be collected predose on dosing days may be collected up to 3 days prior to dosing.
^{w} CPK samples scheduled to be collected predose on dosing days may be collected up to 3 days prior to dosing.
^{x} Viral serology tests include HCV antibody, HBsAg (HBV surface antigen) and HBV core antibody.
^{y} For WOCBP a serum pregnancy test must be done at screening. A urine pregnancy test may be performed at other visits (−3 day window). Women age < 60 years and amenorrheic for 12 months or more, collect FSH and estradiol at Screening to ensure values are in the postmenopausal range.
^{z} PK sampling times and procedures.
^{aa} Presence of Anti-Drug Antibody should be assessed prior to each dose; EOT.
^{bb} Whole blood for correlative studies to be taken pre-dose and 24-hrs (±2 hrs) post dose.
^{cc} Whole blood for CAR phenotyping to be taken pre-dose, 6-hr (±20 min) and 24-hr (±2 hrs) post dose.
^{dd} Whole blood for cfDNA sample to be taken before each of the first 3 doses (ie, pre-dose C1D1, C1D2, C2D1) and any time an MRI/CT is performed (±7 days).
^{ee} Whole blood for TCR sequencing to be done pre-dose only.

End of Treatment: The EOT visit will be conducted 14 days after administration of the last dose of study drug or within 7 days of decision of EOT in the event of premature study termination. All assessments will be performed as depicted in Table 11A-11C.

Long term follow up occurs after completing the EOT evaluation, follow-up continues for patients who have not progressed or have not started a new line of therapy with exams including CT or MRI studies performed every 3 months for up to 2 years or until progression of disease or until a new line of therapy is started.

Follow-up is also required at least every 3 months until resolution or stabilization for any treatment-related abnormalities that warrant continued monitoring in patients who progressed and otherwise would have completed the study.

Discontinuation of subsequent infusions of individuals can take place following an infusion for any Grade 4 toxicity, Grade 3 DLT or any Type III hypersensitivity reaction. For any Grade 3 non-DLT toxicity, the study can be halted, and resumed at reduced dose (e.g., 50% reduced dose) once toxicity is resolved. If Grade 3 toxicity reoccurs, further infusions will be permanently discontinued.

The Study Review Committee (SRC) will be notified and convened upon finding of DLT, and further administration to the subject halted. Following review, the SRC may recommend whether to:
Continue the study as planned, including administration of the additional doses
Expand the dose cohort to obtain additional safety information
Decrease the dose either to a previous lower dose or to an intermediate dose
Eliminate administration of additional doses
Terminate the study and follow all participants for safety

Administration of the drug will be halted if there is evidence of infection, if any toxicity has not resolved to less than or equal to Grade 1 by CTCAE criteria, or if there is not adequate end organ function defined by the following lab parameters:

Creatinine clearance >45 mL/min or within 10% of baseline value as calculated using the Chronic Kidney Disease Epidemiology Collaboration (CKD-EPI) equation or creatinine <1.5×upper limit of normal (ULN) or within 10% of baseline value.

Alanine aminotransferase (ALT)<3.0×ULN; ALT<5×ULN if known liver disease. Note: if known liver disease and ALT≥5×ULN but ≤Grade 1 by CTCAE (≤3.0×baseline (pre-dose) if baseline was abnormal) then dosing can be considered in discussion with the Medical Monitor.

Aspartate transaminase (AST)<3.0×ULN; AST<5×ULN if known liver disease. Note: if known liver disease and AST≥5×ULN but ≤Grade 1 by CTCAE (≤3.0×baseline (pre-dose) if baseline was abnormal) then dosing can be considered in discussion with the Medical Monitor.

Serum bilirubin <1.5×ULN (<3×ULN in participants with documented Gilbert's syndrome).

Enrollment will be paused and regulatory authority notified, if there is occurrence of death not related to disease progression within 30 days of receiving any dose of the DP in any one cohort; or if there is occurrence of two Grade 4 DLTs in two participants in any cohort.

Performance Status: Performance status will be measured using the ECOG performance scale (Oken, 1982) as per Table 12.

TABLE 12
Eastern Cooperative Oncology Group Performance Status

Grade

| | |
|---|---|
| 0 | Fully active, able to carry on all pre-disease performance without restriction |
| 1 | Restricted in physical strenuous activity but ambulatory and able to carry out work of |
| | a light or sedentary nature, eg, light housework, office work |
| 2 | Ambulatory and capable of all self-care but unable to carry out any work activities; up |
| | and about more than 50% of waking hours |
| 3 | Capable of only limited self-care; confined to bed or chair more than 50% of waking |
| | hours |
| 4 | Completely disabled; cannot carry on any self-care; totally confined to bed or chair |

Clinical Safety Assessments: Participants will be assessed for AEs and graded according to the CTCAE version 5.0 grading scale. All AEs must be recorded in the eCRF.

Clinical Laboratory Tests: Clinical laboratory tests will be performed by a qualified local laboratory. Blood and urine samples will be collected for clinical chemistry, hematological analysis, urinalysis, creatine phosphokinase (CPK), ferritin, INR/PTT, thyroid function (including but not limited to thyroid stimulating hormone (TSH), free T4, T3), lipid profile (including but not limited to cholesterol, high-density lipoprotein (HDL) and low-density lipoprotein (LDL)), fibrinogen, tumor markers (cancer-subtype specific), and IL-6 (if Participant experiences a fever ≥Grade 2) according to the clinical research unit's laboratory standard operating procedures (SOPs) at the time points indicated in Tables 11A-11C. Selected clinical laboratory analytes will be measured throughout the study (Table 13)

TABLE 13
Clinical Laboratory Analytes

| | |
|---|---|
| Hematology^{a}: | CBC with differential including: Hematocrit, hemoglobin, red blood cell |
| | (RBC) count, white blood cell (WBC) count, differentials (neutrophils, |
| | cosinophils, basophils, lymphocytes, and monocytes), platelet count, and |
| | reticulocytes |
| Serum chemistry^{a}: | Albumin, alkaline phosphatase, ALT (SGPT), AST (SGOT), GGT, |
| | bicarbonate, urea, calcium, chloride, creatinine, glucose, lipase, |
| | potassium, sodium, magnesium, phosphorous, total bilirubin, total protein |
| Urinalysis^{a}: | Color, specific gravity, pH, glucose, ketones, protein, bilirubin, |
| | urobilinogen, WBCs, RBCs, and microscopy |

^{a}= All laboratory assessments may be performed up to 72 hours prior to dosing on dosing days

For WOCBP, pregnancy tests will be performed according to the method specified and at the timepoints indicated in Tables 11A-11C. If screening pregnancy test is positive, the participant should not be enrolled. If pregnancy test is positive during the treatment period, the participant should not receive further doses of the DP. If a pregnancy test is positive any time during the study the Medical Monitor must be notified immediately. If a female partner of a male participant becomes pregnant during the conduct of the study, it must also be reported by contacting the Medical Monitor for instructions.

Tumor markers include the following:
CA125 for patients with ovarian cancer,
CEA for patients with colorectal cancer,
CA19.9 for patients with pancreatic cancer, and
CA15.3 for patients with advanced breast cancer.

Assessment of these biomarkers is required for participants with the specified diagnosis on (or up to 3 days prior to) Cycle 1/Dose 1 (CID1) and may be assessed for other tumor types at the discretion of the Investigator. If the baseline measurement is within normal range on CID1, no further testing is required. If the baseline measurement is outside the normal range, collection of additional samples is required at the visits shown in Tables 11A-11C. For samples collected on dosing days, the collection window is-3 days; for samples collected on imaging days, the collection window is +7 days. All samples are performed locally and may be performed up to 3 days prior to dosing when scheduled for a dosing day. In addition, all biomarkers may be assessed as clinically indicated throughout the study.

Laboratory reports will be reviewed, signed, and dated by the Investigator or delegated physician. The Investigator will comment on out-of-range parameters and assess clinical significance.

Electrocardiogram: A baseline 12-lead ECG will be obtained after the participant has been rested in a supine position for at least 5 minutes. All 12-lead ECGs should be recorded while the participant is in the supine position. The following parameters will be collected or calculated: ventricular rate (beats per minute [bpm]), PR interval, QRS duration, QT interval, corrected QT interval using Fridericia's calculation (QTcF), and/or corrected QT interval using Bazett's calculation (QTcB). An ECG may be repeated as clinically indicated throughout the study.

Echocardiogram/MUGA: An ECHO or multi-gated acquisition (MUGA) will be performed at screening to determine eligibility. Left ventricular function will be assessed prior to study entrance and will be recorded in the eCRF. Additional assessments may be performed if clinically indicated. ECHO/MUGA performed within 30 days prior to Screening and after last line of therapy is sufficient for Screening criteria.

Tumor response: Screening (Baseline) MRI/CT scans of the neck, chest, abdomen, and pelvis, along with the appropriate imaging of all other sites of disease are required. Follow-up MRI/CT will be performed per study assessments. On-study scans may be collected in original DICOM format and held in the eCRF for possible independent review and analysis. cfDNA will be performed per the Schedule of Events. A bone scan is required at Screening in Participants with previously documented bone metastases and in Participants with advanced breast cancer. The bone scan should be performed prior to the MRI/CT. Additional bone scans should be performed under the following circumstances:
If bone metastases are present, a bone scan should be performed prior to the MRI/CT at each visit where an MRI/CT is conducted (see Tables 11A-11C);
If symptoms of bone lesions are present, a bone scan should be conducted as soon as possible.
If the presence of bone lesions is confirmed, a bone scan should be performed prior to the MRI/CT at each subsequent visit where an MRI/CT is performed.

### Central Laboratory Assessments:

Correlative Studies and Research: Correlative studies and research assays will be performed during the trial to monitor the biological parameters that may impact anti-TROP2 CAR-LNP treatment outcomes. Blood and tumor tissue will be sent to a central lab for analyses. Such studies include the treatment-related effects of cytokine and chemokine production, TCR expansion, and cell phenotype in blood. In addition, they will include treatment-related effects to tumor architecture and cell phenotype.

Pharmacokinetics: Blood will be collected at the visits indicated in Tables 11A-11C. Times of collection for PK blood samples with acceptable time windows are shown below in Table 14 (Cohorts 1-4) and Table 15 (Cohorts 5-7). Samples will be collected and shipped according to instructions provided in the laboratory manual. A central laboratory will access PK parameters of DP in blood using real-time quantitative polymerase chain reaction (RT-qPCR) to detect anti-TROP2 CAR mRNA and mass spectrometry to detect the lipids. Note that blood will be collected using standard site practices with the caveat that the sampling site must be distal from the infusion site.

TABLE 14
Pharmacokinetic Sampling Schedule for Cohorts 1-4

| | Doses 1 & 3 | | | | | | | | | | | Doses 2, 4-14¹ | |
|---|---|---|---|---|---|---|---|---|---|---|---|---|---|
| | Time² | | | | | | | | | | | | |
| | Pre- | 30 | | | | | | | | | 7 | Pre- | |
| | dose | min | 1 hr | 1.5 hr | 2 hr | 3 hr | 5 hr | 7 hr | 24 hr | 48 hr | days³ | dose | 7 hr |
| | Window⁴ | | | | | | | | | | | | |
| | −10 | ±5 | ±5 | ±5 | ±10 | ±10 | ±10 | ±20 | | | ±1 | −10 | ±20 |
| | min | min | min | min | min | min | min | min | ±2 hr | ±2 hr | day | min | min |
| Anti- | X | X | X | X | X | X | X | X | X | X | X | X | X |
| TROP2 | | | | | | | | | | | | | |
| CAR mRNA | | | | | | | | | | | | | |
| Lipid 1 | X | X | X | X | X | X | X | X | X | X | X | X | X |
| Lipid 2 | X | X | X | X | X | X | X | X | X | X | X | X | X |

¹Cycles 5-12 (Doses 7-14) will only be administered based on response to treatment
²Time 0 is the start of infusion.
³Cohorts 1-3 only. Based on emerging PK data, collection of the 7-day post-dose sample may not be required and visit on D22 may not be required. The site will be notified by the Sponsor if this visit is not needed. Note: No 7 day PK collection occurs in Cohort 4.
⁴The actual time for each blood draw must be accurately recorded.

TABLE 15
Pharmacokinetic Sampling Schedule for Cohorts 5-7 (Q14D Dosing Regimen)

| | | | | | | | | | | | | Cycles 1&2: | |
|---|---|---|---|---|---|---|---|---|---|---|---|---|---|
| | | | | | | | | | | | | Dose 2 and | |
| | Cycles 1 & 2: Dose 1 | | | | | | | | | | | Cycles 3+¹ | |
| | Time² | | | | | | | | | | | | |
| | Pre- | 30 | | 1.5 | | | | | | | 7 | Pre- | |
| | dose | min | 1 hr | hr | 2 hr | 3 hr | 5 hr | 7 hr | 24 hr | 48 hr | days³ | dose | 7 hr |
| | Window⁴ | | | | | | | | | | | | |
| | −10 | ±5 | ±5 | ±5 | ±10 | ±10 | ±10 | ±20 | | | ±1 | −10 | |
| | min | min | min | min | min | min | min | min | ±2 hr | ±2 hr | day | min | ±20 min |
| Anti- | X | X | X | X | X | X | X | X | X | X | X | X | X |
| TROP2 | | | | | | | | | | | | | |
| CAR | | | | | | | | | | | | | |
| mRNA | | | | | | | | | | | | | |
| Lipid 1 | X | X | X | X | X | X | X | X | X | X | X | X | X |
| Lipid 2 | X | X | X | X | X | X | X | X | X | X | X | X | X |

¹Samples for PK analysis will be collected through Cycle 3; additional PK samples (one predose and/or one up to 7 hr postdose) may be collected in later cohorts based on emerging PK data.
²Time 0 is the start of infusion.
³Based on emerging PK data, collection of the 7-day post-dose sample may not be required. The site will be notified by the Sponsor if this visit is not needed.
⁴The actual time for each blood draw must be accurately recorded.

### Anti-Drug Antibodies (ADA) to the Drug Product (DP)

Serum samples will be sent to a central laboratory to assess for ADAs throughout the study. Samples will be analyzed for ADAs specific to TROP2 CAR and PEG.

Tumor Biopsies: A fresh tumor biopsy is preferred during the screening period; however, an archived biopsy may be used after discussion with the Medical Monitor. Other protocol-defined biopsies are optional for Cohorts 1 and 2. Platelet support may be provided per institutional standard of care. Each biopsy will be performed according to instructions provided in the Study Laboratory Manual, and tissue will be sent to a central laboratory for analysis. If a local pathology assessment is done, please record results in the eCRF. An optional biopsy may be performed at any time throughout the study at the discretion of the Investigator. A biopsy should not be performed during the study if the Investigator determines a biopsy would be associated with unnecessary risk to the patient. A discussion with the Medical Monitor should occur if the biopsy cannot be performed on the specified day in Tables 11A-11C. Peripheral Anti-TROP2 CAR Expression: Blood will be collected at the time points indicated in Tables 11A-11C to assess for the expression of anti-TROP2 CAR expression in the peripheral blood throughout the study.

Cytokine Levels: Blood will be collected at the time points indicated in Tables 11A-11C to assess the levels of, but not limited to, the following: IL-1-b, IL-2, IL-6, IP-10, MCP-1, MIP-1b, IFN-g, IFN-a, TNF-a, IL-10, GM-CSF, IL-8, IL-4, IL-IRA, IL-12p70, IL-13, and IL-18. If DLT criteria are met, cytokine assessment will be performed.

Complement Levels: Blood will be collected at the time points indicated in Tables 11A-11C to assess the levels of complement, including but not limited to C5b-9.

Total Blood Volume: The amount of blood to be drawn from participants is solely for research purposes and will be limited to that needed to meet the goals of the research and will not exceed the recommended limits. These limits have been set by participant body weight. The amount of blood to be drawn for this clinical trial will not exceed 200 mL/day and a maximum of 350 mL in a 4-week period.

Adverse Events (AE): An AE is any untoward medical occurrence in a participant administered a pharmaceutical product that does not necessarily have to have a causal relationship with the pharmaceutical product. An AB can, therefore, be any unfavorable and unintended sign, symptom, or disease temporally associated with the use of a medicinal product, whether related to the medicinal product. Any worsening of the patient's disease under study or other medical conditions will also be an AE, unless it is within the normal range of disease fluctuation for that patient.

Clinically meaningful (for a given patient) changes in physical examination findings and abnormal objective test findings (e.g, clinical laboratory tests, ECG, or vital signs) should also be recorded as AEs. The criteria for determining whether an abnormal objective test finding should be reported as an AE are as follows:

Test result is associated with accompanying symptoms or is of clinical concern.

Test result requires additional diagnostic testing or medical and/or surgical intervention.

Test result leads to a change in study dosing or discontinuation from the study, significant additional concomitant drug treatment, or other therapy.

Merely repeating an abnormal test, in the absence of any of the above conditions, does not meet the second condition above for reporting as an AE.

Any abnormal test result that is determined to be an error does not require reporting as an AE.

The term “disease progression” as assessed by measurement of malignant lesions on radiographs or other methods should not be reported as AEs. Death due to disease progression in the absence of signs and symptoms should be reported as the primary tumor type.

Adverse Events of Special Interest: AESIs for this protocol are as follows:
≥Grade 2 Infusion Reaction (IR)
≥Grade 3 CRS
≥Grade 3 ICANS
Any ≥ Grade 3 Hypersensitivity reaction
Any second primary malignancy (defined as a new primary malignancy or recurrence of a pre-existing malignancy other than the indication disease), will be reported for the duration of the study, regardless of severity or causal relationship, and these will be also collected throughout long-term follow-up period.

All AESIs, regardless of causality, will be recorded on the AE eCRF and must be reported within 24 hours of the Investigator learning of its occurrence. Additional details of the AESI must be provided via a follow-up AESI form and submitted accordingly. An AESI may or may not be considered an SAE. The events should be recorded in the eCRF through Cycle 12 or until disease progression.

### Classifying Adverse Events

Severity: The severity of the AE will be graded as follows according to CTCAE v5.0 (Table 16):

TABLE 16
Severity of Adverse Events

| | |
|---|---|
| Mild: | Grade 1; Asymptomatic or mild symptoms; clinical or diagnostic observations |
| | only; intervention not indicated |
| Moderate: | Grade 2; Minimal, local or noninvasive intervention indicated |
| Severe: | Grade 3; Medically significant but not immediately life-threatening; |
| | hospitalization or prolongation of hospitalization indicated; disabling |
| Life-threatening: | Grade 4; Urgent intervention indicated |
| Death: | Grade 5; Death related to AE |

The relationship of the AE to study treatment or procedure will be assessed using one of the following terms: 1. Related; 2. Possibly related; 3. Note related

The assessment of causality may be based on factors such as biological plausibility, temporal relationship between product exposure and onset of the event as well as de-challenge (discontinuation of the product to determine if the AE resolves) and rechallenge (reintroduction of the product to determine if the AE recures).

Follow-up of Adverse Events: If any AEs are present when a participant completes the study or if a participant is withdrawn from the study, the participant will be re-evaluated until the AE is resolved or for at least 30 days after the final dose of study drug. At the investigator's discretion, minor AEs can be re-evaluated via telephone and documented. If the AE has still not resolved, additional follow-up will be performed as appropriate. Every effort is to be made by the Investigator or delegate to contact the participant until the AE has resolved or stabilized or the medical monitor and Investigator agree that further follow-up is not necessary. This should be documented in the patient's medical records.

Serious Adverse Events An SAE is any untoward medical occurrence that, at any dose:
Results in death
Is life-threatening
Requires participant hospitalization or prolongation of existing hospitalization
Results in persistent or significant disability and/or incapacity
Is a congenital anomaly or birth defect

Disease progression, which is expected in this study population and measured as an efficacy endpoint, should not be recorded as an AE or SAE term unless the investigator deems them to be possibly related to the study drug. Disease progression and clinical progression are captured in the clinical database (i.e. tumor assessment, EOT, and EOS eCRFs). Death related to clinical/RECIST disease progression are captured on the Death and EOS eCRFs. If death occurs in the context of disease progression. The disease being studied, or expected progression, sign or symptoms of the disease being studied, unless more severe than expected for the Participant's condition is not considered an SAE.

Life-threatening Adverse Event: The term “life-threatening” in the definition of “serious” refers to an event in which the participant was at risk of death at the time of the event, but it does not refer to an event that, hypothetically, might have caused death if it were more severe. Hospitalization: This is defined as the participant being hospitalized overnight or the patient's hospital stay being prolonged for at least an additional overnight stay. Preplanned hospital stays or hospital stays for nonmedical social reasons will not be considered as hospitalization.

Administration of DP may cause IRs, such as fever, rigors, rash, urticaria, dyspnea, hypotension, and/or nausea. Treatment for the patient's primary malignancy, such as chemotherapy, immunotherapy, targeted agents, radiation, and high dose corticosteroid, other than defined/allowed in this protocol, and other investigational agents are prohibited except as needed for treatment of disease progression after IP administration. Treatment with systemic corticosteroids during the first 2 months of treatment should be avoided except for urgent medical conditions. If steroids are initiated, it should be discussed with the medical monitor within 1 business day. Bisphosphonates and denosumab are allowed for participants with bone metastasis or other diseases as clinically indicated.

1.-86. (canceled)

87. A method of treating a cancer in a subject in need thereof, the method comprising administering to the subject a pharmaceutical composition comprising a nucleic acid encoding a chimeric antigen receptor comprising (i) an extracellular domain comprising an anti-TROP2 binding domain and (ii) a transmembrane domain from CD89, wherein the pharmaceutical composition is formulated for systemic delivery, wherein a therapeutically effective amount of the pharmaceutical composition is from about 0.001 mg/kg to about 0.2 mg/kg of the nucleic acid.

88. The method of claim 87, wherein the first cycle of administration comprises administering the therapeutically effective amount to the subject every 7 days or every 14 days.

89. The method of claim 87, wherein the second cycle of administration comprises administering the therapeutically effective amount to the subject every 28 days.

90. The method of claim 87, wherein the pharmaceutical composition is administered to the subject every week for two weeks.

91. The method of claim 87, wherein the pharmaceutical composition is administered to the subject once every three weeks.

92. The method of claim 87, wherein the pharmaceutical composition is administered to the subject every week for two weeks, followed by once every three weeks.

93. The method of claim 87, wherein the therapeutically effective amount is from about 0.005 mg/kg to about 0.2 mg/kg.

94. The method of claim 87, wherein the therapeutically effective amount is from about 0.01 mg/kg to about 0.2 mg/kg.

95. The method of claim 87, wherein the therapeutically effective amount is from about 0.03 to about 0.15 mg/kg.

96. The method of claim 87, wherein the method further comprises administering to the subject a checkpoint inhibitor.

97. The method of claim 87, wherein the nucleic acid is an mRNA.

98. The method of claim 97, wherein the mRNA is an unmodified mRNA.

99. The method of claim 87, wherein the anti-TROP2 binding domain comprises
(a) a heavy chain comprising:
i. an HCDR3 having a sequence of GGFGSSYWYFDV,
ii. an HCDR2 having a sequence of WINTYTGEPTYTDDFKG, and
iii. an HCDR1 having a sequence of NYGMN;
(b) a light chain comprising:
i. an LCDR3 having a sequence of QQHYITPLT,
ii. an LCDR2 having a sequence of SASYRYT, and
iii. an LCDR1 having a sequence of KASQDVSIAVA.

100. The method of claim 87, wherein the CD89 transmembrane domain comprises a sequence of SEQ ID NO: 11.

101. The method of claim 87 wherein the extracellular domain comprises a sequence from CD89, wherein the sequence from CD89 comprises a sequence of SEQ ID NO: 8.

102. The method of claim 87, wherein the chimeric antigen receptor comprises an intracellular domain from CD89, wherein the intracellular domain from CD89 comprises a sequence of SEQ ID NO: 12.

103. The method of claim 99, wherein the anti-TROP2 binding domain comprises a sequence of SEQ ID NO: 3, wherein the extracellular domain comprises a sequence of SEQ ID NO: 8, wherein the transmembrane domain from CD89 comprises a sequence of SEQ ID NO: 11, and wherein the chimeric antigen receptor comprises an intracellular domain comprising a sequence of SEQ ID NO: 12.

104. The method of claim 87, wherein the nucleic acid is encapsulated in a lipid nanoparticle.

105. The method of claim 104, wherein the lipid nanoparticle comprises a cationic lipid, a non-cationic lipid, a neutral lipid, a PEGylated lipid, or a combination thereof.

106. The method of claim 87, wherein the pharmaceutical composition is formulated for intravenous delivery.

107. The method of claim 87, wherein the cancer is selected from the group consisting of urothelial, cervical, ovarian epithelial, triple-negative breast, HR+/HER2− breast, pancreatic ductal adenocarcinoma, gastric adenocarcinoma, esophageal carcinoma, non-small cell lung and colorectal cancer.


SEQUENCE LISTING (59 sequences)

SEQ ID NO: 1 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 2 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 3 - protein, 244 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 4-6 - skipped (3 entries)

SEQ ID NO: 7 - protein, 17 aa, synthetic construct
MWLQSLLLLGTVACSIS

SEQ ID NO: 8 - protein, 11 aa, synthetic construct
DSIHQDYTTQN

SEQ ID NO: 9 - protein, 18 aa, synthetic construct
SGGGGAAADYKDDDDKGS

SEQ ID NO: 10 - protein, 29 aa, synthetic construct
SGGGGAAADYKDDDDKGSDSIHQDYTTQN

SEQ ID NO: 11 - protein, 19 aa, synthetic construct
LIRMAVAGLVLVALLAILV

SEQ ID NO: 12 - protein, 41 aa, synthetic construct
ENWHSHTALNKEASADVAEPSWSQQMCQPGLTFARTPSVCK

SEQ ID NO: 13 - protein, 46 aa, synthetic construct
LYCRRLKIQVRKAAITSYEKSDGVYTGLSTRNQETYETLKHEKPPQ

SEQ ID NO: 14 - protein, 45 aa, synthetic construct
LYCRLKIQVRKAAITSYEKSDGVYTGLSTRNQETYETLKHEKPPQ

SEQ ID NO: 15 - protein, 42 aa, synthetic construct
RLKIQVRKAAITSYEKSDGVYTGLSTRNQETYETLKHEKPPQ

SEQ ID NO: 16 - protein, 43 aa, synthetic construct
RRLKIQVRKAAITSYEKSDGVYTGLSTRNQETYETLKHEKPPQ

SEQ ID NO: 17 - protein, 35 aa, synthetic construct
YEDMRGILYAAPQLRSIRGQPGPNHEEDADSYENM

SEQ ID NO: 18 - protein, 62 aa, synthetic construct
KKVAKKPTNKAPHPKQEPQEINFPDDLPGSNTAAPVQETLHGCQPVTQEDGKESRISVQERQ

SEQ ID NO: 19 - protein, 255 aa, synthetic construct
MACTGPSLPSAFDILGAAGQDKLLYLKHKLKTPRPGCQGQDLLHAMVLLKLGQETEARISLEALKADAVARLVARQWAGVDSTEDPEEPPDVSWAVARLYHLLAEEKLCPASLRDVAYQEAVRTLSSRDDHRLGELQDEARNRCGWDIAGDPGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSW

SEQ ID NO: 20 - protein, 235 aa, synthetic construct
GSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 21 - protein, 236 aa, synthetic construct
RGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 22 - protein, 393 aa, synthetic construct
GSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQD

SEQ ID NO: 23 - protein, 106 aa, synthetic construct
KKVAKKPTNKAPHPKQEPQEINFPDDLPGSNTAAPVQETLHGCQPVTQEDGKESRISVQERQGSRLKIQVRKAAITSYEKSDGVYTGLSTRNQETYETLKHEKPPQ

SEQ ID NO: 24 - protein, 279 aa, synthetic construct
RLKIQVRKAAITSYEKSDGVYTGLSTRNQETYETLKHEKPPQGSGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 25 - protein, 79 aa, synthetic construct
RLKIQVRKAAITSYEKSDGVYTGLSTRNQETYETLKHEKPPQGSYEDMRGILYAAPQLRSIRGQPGPNHEEDADSYENM

SEQ ID NO: 26 - protein, 320 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGSGGSDSIHQDYTTQNLIRMAVAGLVLVALLAILVENWHSHTALNKEASADVAEPSWSQQMCQPGLTFARTPSVCK

SEQ ID NO: 27 - protein, 333 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVENWHSHTALNKEASADVAEPSWSQQMCQPGLTFARTPSVCK

SEQ ID NO: 28 - protein, 334 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQ

SEQ ID NO: 29 - protein, 371 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQGSYEDMRGILYAAPQLRSIRGQPGPNHEEDADSYENM

SEQ ID NO: 30 - protein, 354 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQ

SEQ ID NO: 31 - protein, 398 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQGSRLKIQVRKAAITSYEKSDGVYTGLSTRNQETYETLKHEKPPQ

SEQ ID NO: 32 - protein, 528 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 33 - protein, 571 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQGSGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 34 - protein, 350 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVENWHSHTALNKEASADVAEPSWSQQMCQPGLTFARTPSVCK

SEQ ID NO: 35 - protein, 351 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQ

SEQ ID NO: 36 - protein, 388 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQGSYEDMRGILYAAPQLRSIRGQPGPNHEEDADSYENM

SEQ ID NO: 37 - protein, 371 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQ

SEQ ID NO: 38 - protein, 415 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQGSRLKIQVRKAAITSYEKSDGVYTGLSTRNQETYETLKHEKPPQ

SEQ ID NO: 39 - protein, 545 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 40 - protein, 588 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQGSGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 41 - protein, 337 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGSGGSDSIHQDYTTQNLIRMAVAGLVLVALLAILVENWHSHTALNKEASADVAEPSWSQQMCQPGLTFARTPSVCK

SEQ ID NO: 42 - protein, 12 aa, synthetic construct
GGFGSSYWYFDV

SEQ ID NO: 43 - protein, 9 aa, synthetic construct
QQHYITPLT

SEQ ID NO: 44 - protein, 5 aa, synthetic construct
NYGMN

SEQ ID NO: 45 - protein, 17 aa, synthetic construct
WINTYTGEPTYTDDFKG

SEQ ID NO: 46 - protein, 11 aa, synthetic construct
KASQDVSIAVA

SEQ ID NO: 47 - protein, 7 aa, synthetic construct
SASYRYT

SEQ ID NO: 48 - protein, 71 aa, synthetic construct
DSIHQDYTTQNLIRMAVAGLVLVALLAILVENWHSHTALNKEASADVAEPSWSQQMCQPGLTFARTPSVCK

SEQ ID NO: 49 - protein, 7 aa, synthetic construct
GYTFTNY

SEQ ID NO: 50 - protein, 8 aa, synthetic construct
GYTFTNYG

SEQ ID NO: 51 - protein, 6 aa, synthetic construct
NTYTGE

SEQ ID NO: 52 - protein, 8 aa, synthetic construct
INTYTGEP

SEQ ID NO: 53 - protein, 14 aa, synthetic construct
ARGGFGSSYWYFDV

SEQ ID NO: 54 - protein, 6 aa, synthetic construct
QDVSIA

SEQ ID NO: 55 - RNA, 1000 nt, synthetic construct
  source 1..1000: mol_type = other RNA
  misc_difference 1..1000: note = This sequence may encompass 200-1000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 56 - protein, 30 aa, synthetic construct
DSIHQDYTTQNLIRMAVAGLVLVALLAILV

SEQ ID NO: 57 - protein, 15 aa, synthetic construct
GGGGSGGGGSGGGGS

SEQ ID NO: 58 - protein, 5 aa, synthetic construct
GSGGS

SEQ ID NO: 59 - protein, 122 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSG